(19) United States
(12) Patent Application Publication
(10) Pub. No.: US 2023/0203499 A1
(43) Pub. Date: Jun. 29, 2023

(54) SYSTEMS AND METHODS FOR TREATING LEVODOPA DYSKINESIA, ENHANCING MOTOR BENEFIT, AND DELAYING DISEASE PROGRESSION

(71) Applicants: Dignity Health, San Francisco, CA (US); Board of Trustees of Michigan State University, East Lansing, MI (US)

(72) Inventors: Fredric Manfredsson, San Francisco, CA (US); Kathy Steece-Collier, East Lansing, MI (US)

(21) Appl. No.: 17/998,128

(22) PCT Filed: May 6, 2021
(86) PCT No.: PCT/US2021/031167

§ 371 Date: Nov. 7, 2022

Related U.S. Application Data
(60) Provisional application No. 63/020,849, filed on May 6, 2020.

(51) Int. Cl. C12N 15/113; C12N 15/86; A61P 25/28; A61P 25/16

(52) U.S. Cl. CPC C12N 15/1138; C12N 15/86; A61P 25/28; A61P 25/16; C12N 2310/14; C12N 2310/531; C12N 2750/14143; C12N 2750/14171

(57) ABSTRACT

Disclosed are systems and compositions for reducing the expression of a CaV1.3 protein in a subject and methods of using the systems for treating dyskinesias induced by DA agonist therapy including levodopa-induced dyskinesias (LID), improving the response to levodopa, and improving the response to levodopa in a subject in need thereof; and slowing progression of Parkinson's disease by providing protection against death or dysfunction of substantia nigra dopamine neurons.

### CROSS-REFERENCE TO RELATED APPLICATIONS

This application claims priority from Provisional Application No. 63/020,849, filed May 6, 2020, the entire contents of which are hereby incorporated by reference.

### GOVERNMENTAL RIGHTS

This invention was made with government support under NS110398 and NS090107 awarded by the National Institutes of Health. The government has certain rights in the invention.

### SEQUENCE LISTING

This application contains a Sequence Listing that has been submitted in ASCII format via EFS-Web and is hereby incorporated by reference in its entirety. The ASCII copy is named 681116_Sequence-Listing_ST25.txt, and is 56 kilobytes in size.

### FIELD OF THE INVENTION

The present disclosure provides systems and methods for treating dyskinesias resulting from dopamine (DA) agonist therapies, and improving the response to levodopa in a subject in need thereof.

### BACKGROUND OF THE INVENTION

Symptomatic treatment of individuals with Parkinson's disease (PD) includes dopamine (DA) replacement therapies with the DA precursor, levodopa, being the gold standard. However, long-term levodopa therapy is associated with motor complications including levodopa-induced dyskinesias (LIDs), and often-debilitating side effects. Dyskinesia is uncontrolled, involuntary movement that may occur with long-term levodopa use and longer time with Parkinson's. Dyskinesia can look like fidgeting, writhing, wriggling, head bobbing or body swaying, and tends to occur most often during times when other Parkinson's symptoms, such as tremor, slowness and stiffness, are well controlled. Although up to 90% of individuals with PD develop this side effect, uniformly effective and well-tolerated anti-dyskinetic treatment remains a significant unmet need. Clinically available drugs that can address LID symptoms are not adequately potent and have only partial and transient impact, a terrible prospect especially for younger individuals with PD that will be using dopamine-replacement therapy for the longest time.

Therefore, as no disease-modifying PD therapies are available, and side effects limit long-term benefits of current symptomatic therapies, an unmet need exists for neuroprotection and LID therapy that maintains motor benefits of levodopa while avoiding this often-debilitating side effect.

### SUMMARY OF THE INVENTION

One aspect of the present disclosure encompasses an engineered genetic system for reducing the expression of a calcium channel, voltage-dependent, L type, alpha 1D subunit (CaV1.3) protein in a target cell. The system can provide continuous, high-potency, and target-selective mRNA-level silencing of striatal CaV1.3 channels.

The system comprises a protein expression modification system engineered to reduce the expression of the CaV1.3 protein or a nucleic acid construct encoding the protein expression modification system. The system also comprises a nucleic acid delivery system for delivering the protein expression modification system or the nucleic acid construct encoding the protein expression modification system to the target cell. The system can selectively reduce the expression of the CaV1.3 protein in the target cell relative to a control cell, without reducing the expression of a CaV1.2 protein. The CaV1.3 protein can have an amino acid sequence encoded by a nucleotide sequence of a human Cacna1d gene, and the system can reduce expression of the CaV1.3 protein by about 20% to about 99%. In some aspects, the target cell is a striatal medium spiny neuron (MSN), a nigral neuron, or combinations thereof.

The protein expression modification system can comprise an interfering nucleic acid molecule having a nucleotide sequence complementary to a target sequence within a gene encoding the CaV1.3 protein. The interfering nucleic acid molecule can be selected from an antisense molecule, siRNA molecules, single-stranded siRNA molecules, miRNA molecules, piRNA molecules, lncRNA molecules, and shRNA molecules. In some aspects, the interfering nucleic acid molecule is a shRNA. When the interfering nucleic acid molecule is an shRNA, the shRNA molecule can comprise a nucleotide sequence complementary to a target sequence within a gene encoding the CaV1.3 protein. The sequence within a gene encoding the CaV1.3 protein can be selected from is selected from SEQ ID NO: 1 to SEQ ID NO: 8, and the shRNA can comprise a nucleotide sequence selected from SEQ ID NO: 9, SEQ ID NOs: 14-19, or any combination thereof.

The protein expression modification system can comprise a nucleic acid expression construct for expressing an shRNA sequence targeting a sequence within a gene encoding the CaV1.3 protein, wherein the expression construct comprises a nucleotide sequence encoding an shRNA molecule operably linked to a promoter. The promoter can comprise a nucleotide sequence encoding an H1 promoter. In some aspects, the nucleic acid expression construct comprises a nucleotide sequence having about 75% or more, 85% or more, 95% or more, or 100% sequence identity with a sequence selected from SEQ ID NO: 21-SEQ ID NO: 26.

The nucleic acid delivery system can comprise a vector. In some aspects, the nucleic acid delivery system comprises a viral vector.

In some aspects, the engineered system comprises a recombinant adeno-associated virus (rAAV) vector encapsidating a nucleic acid construct encoding the protein expression modification system for delivering the expression system to the target cell. The engineered system can also comprise an rAAV vector encapsidating a nucleic acid expression construct comprising a promoter operably linked to a nucleotide sequence encoding an shRNA comprising a nucleotide sequence complementary to a target sequence within a gene encoding the CaV1.3 protein, wherein the rAAV vector comprises an AAV9 capsid, and wherein the promoter comprises a nucleotide sequence encoding H1 promoter. In some aspects, the engineered system comprises an AAV vector, wherein the AAV vector comprises a nucleic acid expression construct for expressing an shRNA sequence comprise a nucleotide sequence having about 75% or more, 85% or more, 95% or more, or 100% sequence identity with a nucleotide sequence selected from SEQ ID NO: 27-SEQ ID NO: 32.

The protein expression modification system can comprise a nucleic acid editing system. When the protein expression modification system comprises a nucleic acid editing system, the nucleic acid editing system can be an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, ribozyme, or a programmable DNA binding domain linked to a nuclease domain.

Another aspect of the present disclosure encompasses an engineered vector-mediated system for reducing expression of a CaV1.3 protein in a target cell. The system comprises a nucleic acid expression construct encoding an interfering nucleic acid molecule having a nucleotide sequence complementary to a target sequence within a gene encoding the CaV1.3 protein to reduce the expression of the CaV1.3 protein. The system also comprises an rAAV vector encapsidating the nucleic acid construct for delivering the nucleic acid construct to the target cell. The expression construct can express an shRNA molecule comprising a nucleotide sequence complementary to a target a sequence within a gene encoding the CaV1.3 protein, operably linked to a promoter. The vector can provide continuous, high-potency, and target-selective mRNA-level silencing of striatal CaV1.3 channels.

An additional aspect of the present disclosure encompasses an rAAV vector for reducing the expression of a CaV1.3 protein in a target cell. The vector encapsidates a nucleic acid expression construct for expressing a protein expression modification system engineered to reduce the expression of the CaV1.3 protein. The expression construct can comprise a nucleotide sequence encoding the shRNA operably linked to a promoter for expressing the shRNA sequence, wherein the shRNA targets a sequence within a gene encoding the CaV1.3 protein.

Another aspect of the present disclosure encompasses a method of treating a levodopa-induced dyskinesia (LID) in a subject in need thereof. The method comprises reducing the expression of a CaV1.3 protein in a target cell by administering to the subject a therapeutically effective amount of a composition comprising an engineered genetic system for reducing the expression of a CaV1.3 protein in a target cell. The system can be as described above. The subject can have Parkinson's disease or can be at risk of developing PD. The method can prevent induction of dyskinesia in a subject undergoing DA replacement therapy or expected to undergo DA replacement therapy, can method reduces dyskinesia in a subject undergoing DA replacement therapy, can reverse dyskinesia in a subject undergoing DA replacement therapy, or even eliminate dyskinesia in a subject undergoing DA replacement therapy. The method can also provide continuous, specific, and high-potency treatment of LIDs.

The system can be administered to the subject after a 1-week temporary withdrawal of DA replacement therapy. The target cell can be a striatal medium spiny neuron (MSN), a nigral neuron, or combinations thereof.

Yet another aspect of the present disclosure encompasses a method of improving the response to a DA replacement therapy in a subject in need thereof. The method comprising administering to the subject a therapeutically effective amount of a composition comprising an engineered genetic system administering to the subject a therapeutically effective amount of a composition comprising an engineered genetic system for reducing the expression of a CaV1.3 protein in a target cell. The system can be as described above.

Another aspect of the present disclosure encompasses a method of protecting neurons from damage in a subject in need thereof. The method comprises administering to the subject a therapeutically effective amount of a composition comprising an engineered genetic system for reducing the expression of a CaV1.3 protein in a target cell. The system can be as described above.

An additional aspect of the present disclosure encompasses a method of slowing progression of Parkinson's disease by providing protection against death or dysfunction of substantia nigra dopamine neurons. The method comprises administering to the subject a therapeutically effective amount of a composition comprising an engineered genetic system for reducing the expression of a CaV1.3 protein in a target cell. The system can be as described above.

Yet another aspect of the present disclosure encompasses one or more nucleic acid constructs encoding any engineered genetic system as disclosed herein, for reducing the expression of a CaV1.3 protein in a target cell.

One aspect of the present disclosure encompasses a cell comprising an engineered genetic system, engineered vector-mediated system, or an rAAV vector as described herein, for reducing the expression of a CaV1.3 protein in the cell. The genetic system, the vector mediated system, and the rAAV vector can be as described herein above.

Another aspect of the present disclosure encompasses a kit comprising one or more engineered systems, one or more nucleic acid constructs encoding engineered systems, engineered vector-mediated systems, rAAV vectors or combinations thereof. The engineered systems, nucleic acid constructs encoding engineered systems, engineered vector-mediated systems, and rAAV vectors, can be used for reducing the expression of a CaV1.3 protein in a target cell. The engineered systems, nucleic acid constructs encoding engineered systems, engineered vector-mediated systems, and rAAV vectors can be as described herein above.

An additional aspect of the present disclosure encompasses use of one or more engineered systems, or vectors for the treatment or prevention of neuronal damage in a subject, for improving the response to a DA replacement therapy, or for slowing progression of Parkinson's disease. The engineered systems and vectors can be as described herein above.

### BRIEF DESCRIPTION OF THE FIGURES

FIG. 1A. LID prevention study. Treatment timeline.

FIG. 1B. LID prevention study. Peak dose LID severity (80 minutes post-levodopa) across time and doses. Statistics: Friedman and Kruskal-Wallis tests with Dunn's multiple-comparison post hoc as shown in graphs. Scr, rAAV-Scrambled-shRNA (n=7); CaV, rAAV-CaV1.3-shRNA (n=10).

FIG. 1C. LID prevention study. Daily time course (20-170/200 minutes post-levodopa) for lepodova dose of 6 mg/kg. The top graphs reflect mean±SEM; bottom graphs show individual subject responses over time. Statistics: Friedman and Kruskal-Wallis tests with Dunn's multiple-comparison post hoc as shown in graphs. Scr, rAAV-Scrambled-shRNA (n=7); CaV, rAAV-CaV1.3-shRNA (n=10).

FIG. 1D. LID prevention study. Daily time course (20-170/200 minutes post-levodopa) for lepodova dose of 9 mg/kg. The top graphs reflect mean±SEM; bottom graphs show individual subject responses over time. Statistics: Friedman and Kruskal-Wallis tests with Dunn's multiple-comparison post hoc as shown in graphs. Scr, rAAV-Scrambled-shRNA (n=7); CaV, rAAV-CaV1.3-shRNA (n=10).

FIG. 1E. LID prevention study. Daily time course (20-170/200 minutes post-levodopa) for lepodova dose of 12 mg/kg. The top graphs reflect mean±SEM; bottom graphs show individual subject responses over time. Statistics: Friedman and Kruskal-Wallis tests with Dunn's multiple-comparison post hoc as shown in graphs. Scr, rAAV-Scrambled-shRNA (n=7); CaV, rAAV-CaV1.3-shRNA (n=10).

FIG. 1F. LID prevention study. Daily time course (20-170/200 minutes post-levodopa) for lepodova dose of 18 mg/kg. The top graphs reflect mean±SEM; bottom graphs show individual subject responses over time. Statistics: Friedman and Kruskal-Wallis tests with Dunn's multiple-comparison post hoc as shown in graphs. Scr, rAAV-Scrambled-shRNA (n=7); CaV, rAAV-CaV1.3-shRNA (n=10).).

FIG. 2A. LID reversibility study. Treatment timeline. All rats received daily high-dose (12 mg/kg) levodopa at all times indicated.

FIG. 2B. LID reversibility study. Peak-dose LID severity (80 minutes post-levodopa) across time. As seen in this depiction of peak LID behaviors on day 53 post-vector, the impact of CaV1.3 channel silencing appears to uniformly reduce all aspects of LID and is not selective on any particular attribute. Data represent the mean±SEM. Statistics: Kruskal-Wallis tests with Dunn's multiple-comparison post hoc as shown in graphs. LD, levodopa; Scr, rAAV-Scrambled-shRNA (n=12); CaV, rAAV-CaV1.3-shRNA (n=11).

FIG. 2C. LID reversibility study. Daily time course of LID behavior ranging from 20 to 200 minutes post-levodopa on each rating day; Days 15, 20, and 25. As seen in this depiction of peak LID behaviors on day 53 post-vector, the impact of CaV1.3 channel silencing appears to uniformly reduce all aspects of LID and is not selective on any particular attribute. Data represent the mean±SEM. Statistics: Kruskal-Wallis tests with Dunn's multiple-comparison post hoc as shown in graphs. LD, levodopa; Scr, rAAV-Scrambled-shRNA (n=12); CaV, rAAV-CaV1.3-shRNA (n=11).

FIG. 2D. LID reversibility study. Daily time course of LID behavior ranging from 20 to 200 minutes post-levodopa on each rating day; Days 31, 36, and 40. As seen in this depiction of peak LID behaviors on day 53 post-vector, the impact of CaV1.3 channel silencing appears to uniformly reduce all aspects of LID and is not selective on any particular attribute. Data represent the mean±SEM. Statistics: Kruskal-Wallis tests with Dunn's multiple-comparison post hoc as shown in graphs. LD, levodopa; Scr, rAAV-Scrambled-shRNA (n=12); CaV, rAAV-CaV1.3-shRNA (n=11).

FIG. 2E. LID reversibility study. Daily time course of LID behavior ranging from 20 to 200 minutes post-levodopa on each rating day; Days 46, 50, and 53. As seen in this depiction of peak LID behaviors on day 53 post-vector, the impact of CaV1.3 channel silencing appears to uniformly reduce all aspects of LID and is not selective on any particular attribute. Data represent the mean±SEM. Statistics: Kruskal-Wallis tests with Dunn's multiple-comparison post hoc as shown in graphs. LD, levodopa; Scr, rAAV-Scrambled-shRNA (n=12); CaV, rAAV-CaV1.3-shRNA (n=11).

FIG. 2F. LID reversibility study. Individual attributes of LID. As seen in this depiction of peak LID behaviors on day 53 post-vector, the impact of CaV1.3 channel silencing appears to uniformly reduce all aspects of LID and is not selective on any particular attribute. Data represent the mean±SEM. Statistics: Kruskal-Wallis tests with Dunn's multiple-comparison post hoc as shown in graphs. LD, levodopa; Scr, rAAV-Scrambled-shRNA (n=12); CaV, rAAV-CaV1.3-shRNA (n=11).

FIG. 3A. Motor response to low-dose (6 mg/kg) levodopa. Data represent total number of rears over a 5-minute test period, prior to or beginning 50 minutes after levodopa. Behavioral responses were examined in all subjects in the LID prevention study. Statistics: 2-way ANOVA with post hoc Sidak's multiple-comparisons test as shown in the graphs; additional comparisons are provided in the Results section. Pre-LD, prelevodopa; post-LD, postlevodopa; Scr, rAAV-Scrambled-shRNA; CaV, rAAV-CaV1.3-shRNA.

FIG. 3B. Motor response to low-dose (6 mg/kg) levodopa. Data represent total number of rears over a 5-minute test period, prior to or beginning 50 minutes after levodopa. Behavioral responses were examined in all subjects in the LID reversibility study. Statistics: 2-way ANOVA with post hoc Sidak's multiple-comparisons test as shown in the graphs; additional comparisons are provided in the Results section. Pre-LD, prelevodopa; post-LD, postlevodopa; Scr, rAAV-Scrambled-shRNA; CaV, rAAV-CaV1.3-shRNA.

FIG. 3C. Motor response to low-dose (6 mg/kg) levodopa. Data represent total number of rotations over a 5-minute test period, prior to or beginning 50 minutes after levodopa. Behavioral responses were examined in all subjects in the LID prevention study. Statistics: 2-way ANOVA with post hoc Sidak's multiple-comparisons test as shown in the graphs; additional comparisons are provided in the Results section. Pre-LD, prelevodopa; post-LD, postlevodopa; Scr, rAAV-Scrambled-shRNA; CaV, rAAV-CaV1.3-shRNA.

FIG. 3D. Motor response to low-dose (6 mg/kg) levodopa. Data represent total number of rotations over a 5-minute test period, prior to or beginning 50 minutes after levodopa. Behavioral responses were examined in all subjects in the LID reversibility study. Statistics: 2-way ANOVA with post hoc Sidak's multiple-comparisons test as shown in the graphs; additional comparisons are provided in the Results section. Pre-LD, prelevodopa; post-LD, postlevodopa; Scr, rAAV-Scrambled-shRNA; CaV, rAAV-CaV1.3-shRNA.

FIG. 4A. CaV silencing and vector distribution. (Panel i) Representative section with GFP immunohistochemistry (IHC) demonstrating the striatal targeting and spread of vector. (Panel ii) Higher-magnification image of that seen in Panel i showing that the GFP that is expressed in the cortex is expressed exclusively in neuritic fibers. (Panel iii) CaV1.3 ISH in the same region as Panel ii, demonstrating that despite neuritic GFP expression in this region in a subject injected with rAAV-CaV1.3-shRNA, there is no impact on cellular CaV1.3 mRNA. Str, striatum.

FIG. 4B. CaV silencing and vector distribution. (Upper plot) Striatal transduction volume. (Bottom plot) Estimate of NeuN-positive cells within the striatum of vector- and non-injected striatum. Statistics: 1-way ANOVA with post hoc Tukey's test. Str, striatum.

FIG. 4C. CaV silencing and vector distribution. CaV1.3 mRNA expression and protein levels. (Panel i) RNAscope in situ hybridization (ISH) showing relative levels of striatal CaV1.3 mRNA expression. (Panel ii) Dual-label immunohistochemistry (IHC) for GFP and CaV1.3. Str, striatum.

FIG. 4D. CaV silencing and vector distribution. Densitometric analysis of CaV1.3 ISH and IHC. CaV1.3 ISH using ImageJ software in the region of the striatum stained positive for GFP IHC in rAAV-CaV1.3-shRNA rats compared with rAAV-Scr-shRNA rats. CaV1.3: unpaired t test. Str, striatum.

FIG. 4E. CaV silencing and vector distribution. Densitometric analysis of CaV1.3 ISH and IHC. CaV1.3 IHC using ImageJ software in the region of the striatum stained positive for GFP IHC in rAAV-CaV1.3-shRNA rats compared with rAAV-Scr-shRNA rats; Mann-Whitney U. Str, striatum.

FIG. 4F. CaV silencing and vector distribution. Densitometric analysis of CaV1.3 ISH and IHC. CaV1.2 ISH using ImageJ software in the region of the striatum stained positive for GFP IHC in rAAV-CaV1.3-shRNA rats compared with rAAV-Scr-shRNA rats; CaV1.2: 1-way ANOVA. Str, striatum.

FIG. 4G. CaV silencing and vector distribution. Nonparametric Spearman correlation analysis of LID severity score for day 10 18 mg/kg versus % inhibition of CaV1.3 mRNA. The dashed line indicates the maximal LID observed in the rAAV-CaV1.3-shRNA rats shown in this graph. Str, striatum.

FIG. 5A. LID Protection against ‘High Dose’ 12 mg/kg Levodopa in Aged Parkinsonian Rats. *=p<0.05, CaV vs Scr, Kruskal Wallis with post-hoc Dunn's multiple comparison. B-C) Mann-Whitney U test. Abbreviations: PV=post-vector; CaV=rAAV-CaV1.3-shRNA; Scr=rAAV-Scr-shRNA; PV=post-vector.

FIG. 5B. LID Protection against ‘High Dose’ 12 mg/kg Levodopa in Aged Parkinsonian Rats. Mann-Whitney U test. Abbreviations: PV=post-vector; CaV=rAAV-CaV1.3-shRNA; Scr=rAAV-Scr-shRNA; PV=post-vector.

FIG. 5C. LID Protection against ‘High Dose’ 12 mg/kg Levodopa in Aged Parkinsonian Rats. Mann-Whitney U test. Abbreviations: PV=post-vector; CaV=rAAV-CaV1.3-shRNA; Scr=rAAV-Scr-shRNA; PV=post-vector.

FIG. 6. CaV1.3 mRNA knockdown in aged macaque monkeys. Each symbol represents a FOV. *p<0.0001 unpaired t-test.

FIG. 7. Timeline for capacity to reverse LID in the NHP striatum.

FIG. 8. Timeline for capacity to prevent LID in the NHP striatum.

### DETAILED DESCRIPTION

The present disclosure is based in part on the discovery of a genetic approach to treating dyskinesias resulting from dopamine agonist therapies such as LID. The systems and methods of the instant disclosure provide a much-needed breakthrough in the treatment of individuals with PD and allows therapies using dopamine (DA) agonists, including the most powerful anti-parkinsonian therapy identified (i.e., levodopa), to work unabated through the duration of the disease. It is believed that the systems and methods described herein provide the most profound anti-dyskinetic benefit reported to date. Unlike pharmacological approaches currently available, the genetic approach described herein provides target-selective, efficient, and uniform prevention of dyskinesias that result from DA agonist therapies. Further, the systems and methods provide complete prevention of levodopa-induced dyskinesias, and this anti-dyskinetic benefit persists long-term, and in the absence of any pharmacological intervention or the off-target side effects associated with these pharmaceuticals. These findings are especially surprising because it was not known that long-term complete prevention of LIDs was even possible. Even more surprising, the systems and methods are also capable of ameliorating or even reversing preexisting severe LIDs and neuroanatomical and molecular indices of LIDs in the striatum. Importantly, motoric responses to levodopa not only remain intact, but are also even improved when compared to levodopa alone, indicating an enhancement of levodopa benefit without dyskinesia liability. Accordingly, the systems and methods described herein have the potential to transform treatment of individuals with PD by allowing maintenance, and even enhancement of the motor benefit of levodopa treatment in the absence of the debilitating levodopa-induced dyskinesia side effect.

The present disclosure is also based in part on the discovery that the systems and methods can slow the progression of Parkinson's disease by providing protection against death or dysfunction of substantia nigra dopamine neurons.

### I. Systems

One aspect of the present disclosure encompasses an engineered genetic system for reducing the expression of a calcium channel, voltage-dependent, L type, alpha 1D subunit (CaV1.3) protein in a target cell. The system comprises a protein expression modification system engineered to reduce the expression of the CaV1.3 protein or a nucleic acid construct encoding the protein expression modification system. The system further comprises a nucleic acid delivery system for delivering the protein expression modification system or the nucleic acid construct encoding the protein expression modification system to the target cell.

(a) LIDs

Levodopa-induced dyskinesias (LIDs) refer to abnormal involuntary behaviors noted in parkinsonian subjects treated with dopamine (DA) agonists, including levodopa and/or other DA agonists such as bromocriptine, cabergoline, pergolide, pram ipexole, ropinirole, rotigotine, apomorphine. There are at least three main types of LIDs, all of which can be treated using the systems and methods of the instant disclosure. Off-period dystonia correlated to the akinesia occurs before the full effect of L-DOPA sets in, when the plasma levels of L-DOPA are low. In general, off-period dystonia occurs as painful spasms in the foot. Another form of LID is diphasic dyskinesia, which occurs when plasma L-DOPA levels are rising or falling. This form occurs primarily in the lower limbs (though they can happen elsewhere) and is usually dystonic (characterized by apparent rigidity within muscles or groups thereof) or ballistic (characterized by involuntary movement of muscles) and will not respond to L-DOPA dosage reductions. A third form of LID is peak-dose dyskinesia, which correlates with the plateau in L-DOPA plasma level. This type usually involves the upper limbs (but could also affect the head, trunk and respiratory muscles), is choreic (of chorea), and less disabling.

Central to the biology of LIDs are changes in synaptic plasticity associated with striatal medium spiny neurons (MSNs), critical targets of convergent cortical glutamate and nigrostriatal DA input. In PD and animal models of PD, striatal DA depletion results in loss of dendritic spines on MSNs, an aberrant feature accompanied by secondary loss of glutamate synapses from corticostriatal projections. The pathognomonic loss of striatal dopamine in PD results in dysregulation and disinhibition of striatal CaV1.3 calcium channels, leading to synaptopathology involved in levodopa-induced dyskinesias. Although there are clinically available drugs that can inhibit CaV1.3 channels, they are not adequately potent and have only partial and transient impact on levodopa-induced dyskinesias. Further, currently available Ca2+ channel blockers cannot selectively inhibit the expression of CaV1.3 without impacting the expression of CaV1.2, a Ca2+ channel important in cardiovascular function. Off-target side effects of these pharmaceuticals could include cardiovascular, learning, memory, and neuroendocrine effects.

A system of the instant disclosure reduces the expression of CaV1.3 in a target cell or tissue. As explained herein above, changes in synaptic plasticity associated with striatal medium spiny neurons (MSNs) are central to the biology of LIDs. Inhibiting the expression of CaV1.3 in the striatal medium spiny neurons using systems and methods of the instant disclosure can treat LIDs. Accordingly, a target cell or tissue of the instant disclosure can be the striatum and/or striatal neurons, including striatal medium spiny neurons. Further, Parkinson's disease is characterized by the loss of dopaminergic neurons in the substantia nigra. Inhibiting CaV1.3 in the substantia nigra can also prevent neurodegeneration that occurs in Parkinsonian subjects. Therefore, a target cell or tissue of the instant disclosure can also be the substantia nigra, or neurons in the substantia nigra.

(b) Protein Expression Modification System

Any protein expression modification system capable of reducing the expression of CaV1.3 protein can be used in the instant disclosure. The nucleic acid modification system can reduce expression of a CaV1.3 protein by about 10% to about 100%, about 5% to about 99%, about 10%, 20%, 30%, 40%, 50%, 60%, 70%, 80%, 85%, 90%, 95%, 96%, 97%, 98%, 99%, or about 100%. It will be recognized that the system can comprise one or more than one programmable nucleic acid modification system to target more than one sequence within a gene encoding the CaV1.3 protein.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. Non-limiting examples of suitable protein expression modification systems include programmable nucleic acid modification systems, or peptide, polypeptide, antibody, or antibody fragments which when expressed in a target cell or tissue type reduce the level of CaV1.3 protein or the calcium channel activity of the protein.

In some aspects, the CaV1.3 protein expression modification system is a programmable expression modification system targeted to a sequence within a gene encoding the CaV1.3 protein. As used herein, a “programmable nucleic acid modification system” is a system capable of targeting and modifying the expression of a nucleic acid sequence to alter a protein or the expression of a protein encoded by the nucleic acid. The programmable nucleic acid modification system can comprise an interfering nucleic acid molecule or a guided protein expression modification system.

In some aspects, the programmable expression modification system comprises an interfering nucleic acid (RNAi) molecule having a nucleotide sequence complementary to a target sequence within a gene encoding the CaV1.3 protein used to inhibit expression of the CaV1.3 protein. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription of the transcript. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs and interfere with signalling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

The protein expression modification system is specifically targeted to a sequence within a gene encoding the CaV1.3 protein. Generally, the target nucleic acid sequence contains negligible overlap with genes encoding CaV1.2 protein. In some aspects, the CaV1.3 protein has an amino acid sequence encoded by a nucleotide sequence of a human Cacna1d gene. In some aspects, the CaV1.3 protein has an amino acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 12. In some aspects, the CaV1.3 protein is encoded by a nucleotide sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 13. The programmable nucleic acid modification system can reduce CaV1.3 mRNA levels by about 10% to about 100%, about 5% to about 99%, about 10%, 20%, 30%, 40%, 50%, 60%, 70%, 80%, 85%, 90%, 95%, 96%, 97%, 98%, 99%, or about 100%.

In some aspects, the programmable nucleic acid modification system targeted to a sequence within a gene encoding the CaV1.3 protein is a shRNA molecule comprising a nucleotide sequence complementary to a target sequence within a gene encoding the CaV1.3 protein. In some aspects, the shRNA molecule comprises a nucleotide sequence complementary to, i.e., targeted to a target sequence within the Cacna1d gene. In some aspects, the shRNA has a nucleotide sequence comprising a portion of sequence complementary to a sequence of the Cacna1d gene selected from Table 1. In some aspects, the shRNA has a nucleotide sequence comprising a portion of sequence complementary to a sequence of the human Cacna1d gene selected from Table 1. In some aspects, the shRNA has a nucleotide sequence of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 14 (GAAGAGGCGCGGCCAAGACTTCAAGAGAGTCTTGGCCGCGCCTCTTC)

TABLE 1

| SEQ ID. NO. | Sequence |
|---|---|
| 1 | CGAGGCAAACTATGCAAGA |
| 2 | ACTATGCAAGAGGCACCAGA |
| 3 | GCGTCAGTGTGTGGAATAT |
| 4 | GGCCATTGCTGTAGACAAT |
| 5 | CGTGCCCTCTTCTGTTTAT |
| 6 | CTCCTCGCCTTTCGAATAT |
| 7 | CCCTGAAGATGATTCTAAT |
| 8 | CCCGCTATTATGAAACTTA |

In some aspects, the shRNA has a nucleotide sequence comprising a portion of sequence complementary to a sequence of the human Cacna1d gene (SEQ ID NO: 12). In some aspects, the shRNA has a nucleotide sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with nucleotide sequences selected from Table 2.

TABLE 2

| SEQ | |
|---|---|
| ID. NO. | Sequence |
| 15 | GCGAGGCAAACTATGCAAGATTCAAGAGATCTTGCATAGTT |
| | TGCCTCG |
| 16 | GAGGAAGGCAAACGAAATA TTCAAGAGA TATTTCGTTTG |
| | CCTTCCTC |
| 17 | GCCCTGAAGATGATTCTAATTTCAAGAGAATTAGAATCATC |
| | TTCAGGG |
| 18 | GTCCTGAACTCCATTATAATTCAAGAGATTATAATGGAGTT |
| | CAGGAC |
| 19 | GAGGATCTAAAGGGCTACTTTCAAGAGAAGTAGCCCTTTAG |
| | ATCCTC |

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

In some aspects, the programmable nucleic acid modification system is a programmable nucleic acid editing system. Such modification systems can be engineered to edit specific DNA or RNA sequences to repress transcription or translation of an mRNA encoded by the gene, and/or produce mutant proteins with reduced activity or stability. Non-limiting examples of programmable nucleic acid editing systems include, without limit, an RNA-guided clustered regularly interspersed short palindromic repeats (CRISPR) system, such as a CRISPR-associated (Cas) (CRISPR/Cas) nuclease system, a CRISPR/Cpf1 nuclease system, a zinc finger nuclease (ZFN) system, a transcription activator-like effector nuclease (TALEN) system, or a system comprising a meganuclease, a ribozyme, or a programmable DNA binding domain linked to a nuclease domain. Other suitable programmable nucleic acid modification systems will be recognized by individuals skilled in the art. Such systems rely for specificity on the delivery of exogenous protein(s), and/or a guide RNA (gRNA) or single guide RNA (sgRNA) having a sequence which binds specifically to a gene sequence of interest. The system components are delivered by a plasmid or viral vector or as a synthetic oligonucleotide. For example, engineered CRISPR systems comprise a gRNA or sgRNA, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Systems such as ZFNs and TALENs rely on customizable sequence-specific DNA-binding domains which are connected to a nonspecific nuclease for target cleavage.

Nucleases that can be used in programmable nucleic acid editing systems include any endonuclease that cleaves phosphodiester bonds within a polynucleotide. An endonuclease may specifically cleave phosphodiester bonds within a DNA polynucleotide. In some embodiments, an endonuclease is a ZFN, a TALEN, a homing endonuclease (HE), a meganuclease, a MegaTAL, or a CRISPR-associated endonuclease. In some aspects, an endonuclease is a RNA-guided endonuclease. In certain aspects, an RNA-guided endonuclease can be a CRISPR nuclease, e.g., a Type II CRISPR Cas9 endonuclease or a Type V CRISPR Cpf1 endonuclease. In some embodiments, an endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or combinations thereof. In some embodiments, an endonuclease may introduce one or more single-stranded breaks (SSBs) and/or one or more double-stranded breaks (DSBs).

In some aspects, the programmable nucleic acid modification system is a CRISPR/Cas tool modified for transcriptional regulation of a locus. In some aspects, the programmable nucleic acid modification system is a CRISPR/Cas transcriptional regulator driven by cell-specific promoters using a catalytically dead effector (dCAS9) to modulate transcription of a chitinase gene encoding a chitinase protein.

The protein expression modification system can be encoded by a nucleic acid construct. Nucleic acid constructs can be as described in Section II herein below.

(c) Delivery Systems

The engineered genetic systems of the instant disclosure comprise a nucleic acid delivery system for delivering the nucleic acid construct to the target cell or tissue. The nucleic acid delivery system can be any system capable of delivering the nucleic acid construct to the target cell or tissue. Non-limiting examples of delivery systems include viral and non-viral constructs, and/or vectors to introduce the programmable nucleic acid modification system into a cell or organism. In some aspects, the delivery system has tropism to a target cell or tissue.

In some aspects, the nucleic acid delivery system comprises a non-viral vector. Non-viral vectors can include plasmids, linear DNA fragments, viruses, bacteriophage, pro-viruses, phagemids, transposons, and artificial chromosomes and the like, that may or may not be able to replicate autonomously or integrate into a chromosome of a host cell. Such vectors can be delivered to a cell or tissue by electroporation, using a variety of means. Suitable delivery means include synthetic oligonucleotides, lipoplexes, polymersomes, polyplexes, dendrimers, inorganic nanoparticles, cell-penetrating peptides, microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, implantable devices, magnetofection, lipofection, impalefection, optical transfection, proprietary agent-enhanced uptake of nucleic acids, proprietary agent-enhanced uptake of nucleic acids, and delivery via liposomes, immunoliposomes, virosomes, or artificial virions.

In some aspects, the nucleic acid delivery system comprises a viral vector. The viral vector can be an adenovirus vector; an adeno-associated virus (AAV) vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector.

In some aspects, the vector is a lentiviral vector. A recombinant lentiviral vector is capable of transducing a target cell with a nucleotide of interest. Once within the cell, the RNA genome from the vector particle is reverse-transcribed into DNA and integrated into the genome of the target cell. The lentiviral vector can be derived from or may be derivable from any suitable lentivirus. The lentiviral vector can be derived from primate or non-primate lentiviruses. Examples of primate lentiviruses include but are not limited to the human immunodeficiency virus (HIV) and the simian immunodeficiency virus (SrV). The non-primate lentiviral group includes the prototype “slow virus” visna/maedi virus (VMV), as well as the related caprine arthritis-encephalitis virus (CAEV), equine infectious anemia virus (EIAV), feline immunodeficiency virus (FIV), and bovine immunodeficiency virus (BIV).

In other aspects, the viral vector is a herpes simplex virus (HSV) vector. The genome of the type-1 (HSV-1) is about 150 kb of linear, double-stranded DNA, featuring about 70 genes. Many viral genes can be deleted without the virus, losing its ability to propagate. The “immediately early” (IE) genes are transcribed first. They encode transacting factors which regulate expression of other viral genes. The “early” (E) gene products participate in replication of viral DNA. The late genes encode the structural components of the virion as well as proteins, which turns on transcription of the IE and E genes, or disrupts host cell protein translation.

In yet other aspects, the delivery system comprises an adeno-associated virus (AAV) encapsidating an rAAV vector comprising a nucleic acid construct encoding the protein expression modification system for delivering the expression system to the target cell. The adenovirus genome consists of about 36 kb of double-stranded DNA. Adenoviruses target airway epithelial cells, but are also capable of infecting neurons. Recombinant adenovirus vectors have been used as gene transfer vehicles for non-dividing cells. These vectors are similar to recombinant HSV vectors, since the adenovirus E1a immediate-early gene is removed, but most viral genes are retained. Since the E1a gene is small (roughly 1.5 kb) and the adenovirus genome is approximately one-third of the size of the HSV genome, other non-essential adenovirus genes are removed in order to insert a foreign gene within the adenovirus genome.

rAAV vectors can be constructed using known techniques to provide at least the operatively linked components of control elements, including a transcriptional initiation region, an exogenous nucleic acid molecule, a transcriptional termination region, and at least one post-transcriptional regulatory sequence. The control elements are selected to be functional in the targeted cell and/or in combination with incorporation of mutations that enhance specific infectivity.

In some aspects, the delivery system has tropism to a desired target cell or tissue type. In some aspects, the target cell or tissue type is a cell of the central nervous system. The use of rAAV vectors to deliver nucleic acids into the brain is well known in the art. (See, e.g., U.S. Pat. No. 8,487,088, which is incorporated by reference herein in its entirety). The AAV can be any AAV serotype, including a serotype selected from AAV1, AAV2, AAV3, AAV4, AAV5, AAV6, AAV7, AAV8, AAVrh8, AAVrh10, AAVrh39, or AAVrh43. In some aspects, the AAV serotype is AAV9. Included are AAV capsid proteins comprising mutations with improved transduction efficiency of a desired target cell type. Non-limiting examples of capsid mutations having improved transduction efficiency include the Y444F, Y500F, Y730F, T491V, R585S, R588T, R487G amino acid substitutions in the AAV2 capsid protein, or corresponding substitutions in the capsid protein of another AAV serotype, in various combinations and/or in combination with incorporation of mutations that enhance tropism of the virus to a desired target cell or tissue type. Such mutations can include insertion of one or more peptides for targeting the virus to a cell or tissue type.

In some aspects, the AAV capsid protein is an AAV2 capsid protein comprising mutations inhibiting the canonical HSPG binding site such as the R585S, R588T, and R487G amino acid substitutions in various combinations, or corresponding substitutions in the capsid protein of another AAV serotype. In some aspects, the AAV capsid protein is an AAV9 capsid comprising mutations corresponding to the R585S, R588T, and R487G amino acid substitutions in the AAV2 capsid in various combinations, or corresponding substitutions in the capsid protein of another AAV serotype. In some aspects, the AAV capsid protein comprises the mutations inhibiting the canonical HSPG binding site such as the Y444F, Y500F, Y730F amino acid substitutions in various combinations, or corresponding substitutions in the capsid protein of another AAV serotype. In some aspects, the AAV capsid protein comprises an AAV9 capsid comprising mutations corresponding to the Y444F, Y500F, Y730F amino acid substitutions in an AAV2 capsid in various combinations, or corresponding substitutions in the capsid protein of another AAV serotype.

In some aspects, the delivery system comprises an AAV vector having tropism or improved efficiency in targeting a cell type in the nervous system. In some aspects, the delivery system comprises an AAV vector having tropism or improved efficiency in targeting striatal neurons. In some aspects, the delivery system comprises an AAV vector having tropism or improved efficiency in targeting striatal medium spiny neurons (MSNs). In some aspects, the delivery system comprises an AAV vector having tropism or improved efficiency in targeting nigral neurons. In some aspects, the delivery system comprises an AAV vector having tropism or improved efficiency in targeting striatal and nigral neurons.

In certain aspects, the engineered system comprises an AAV vector comprising a nucleic acid expression construct for expressing an interfering nucleic acid molecule targeting a sequence within a gene encoding the CaV1.3 protein. In some aspects, the protein expression modification system comprises a nucleic acid expression construct for expressing an shRNA sequence targeting a sequence within a gene encoding the CaV1.3 protein, wherein the expression construct comprises a nucleotide sequence encoding an shRNA molecule operably linked to a promoter. Expression constructs can be as described in Section II herein below. The shRNA molecule comprises a nucleotide sequence complementary to a target sequence within a gene encoding the CaV1.3 protein and can be as described in Section 1(b) herein above. In some aspects, the expression system is cloned into an AAV genome to generate AAV vectors. In some aspects, AAV vectors comprise a nucleic acid expression construct comprising a nucleotide sequence encoding an shRNA molecule operably linked to a promoter. In some aspects, AAV vectors comprising a nucleic acid expression construct for expressing an shRNA sequence comprise a nucleotide sequence having about 75% or more, 85% or more, 95% or more, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleotide sequence selected from SEQ ID NO: 27-SEQ ID NO: 32.

### II. Nucleic Acid Constructs

The protein expression modification system can be encoded by one or more nucleic acid constructs. The expression modification constructs generally comprise DNA coding sequences operably linked to at least one promoter control sequence for expression of the protein modification system in a target cell or tissue. Promotor control sequences can include constitutive, ubiquitous, regulated, cell- or tissue-specific promoters, or combinations thereof.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter. Promoter control sequences can also be promoter control sequences of the gene of interest, such that the expression pattern of the one or more nucleic acid constructs matches the expression pattern of the gene of interest. The promoter sequence can be wild type or it can be modified for more efficient or efficacious expression.

The nucleic acid constructs can comprise additional expression control sequences (e.g., enhancer sequences, Kozak sequences, polyadenylation sequences, transcriptional termination sequences, etc.), selectable reporter sequences (e.g., antibiotic resistance genes), origins of replication, and the like. The nucleic acid constructs can further comprise RNA processing elements such as glycine tRNAs or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector can further comprise sequences for expression of Csy4 RNAse to process the gRNA transcript. Additional information about nucleic acid constructs and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, N.Y., 3rd edition, 2001. Other methods of controlling expression in a specific tissue or target cell can be as described in Section I(c) and Section III.

Nucleic acid constructs encoding an expression modification system can comprise one or more constructs encoding the expression system. The nucleic acid constructs can be DNA or RNA, linear or circular, single-stranded or double-stranded, or any combination thereof. The nucleic acid constructs can be codon optimized for efficient translation into protein in the cell of interest. Codon optimization programs are available as freeware or from commercial sources.

In some aspects, the protein expression system comprises a nucleic acid expression construct for expressing an shRNA sequence targeting a sequence within a gene encoding the CaV1.3 protein, wherein the expression construct comprises a nucleotide sequence encoding an shRNA molecule operably linked to at least one promoter. In some aspects, the at least one promoter control sequence is a tissue or cell-specific promoter control sequence. In some aspects, the promoter comprises a nucleotide sequence encoding a human H1 polymerase III promoter such as a promoter having a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 11 (5′ AATTCATATT TGCATGTCGC TATGTGTTCT GGGAAATCAC CATAAACGTG AAATGTCTTT GGATTTGGGA ATCTTATAAG TTCTGTATGA GACCACTCGG ATCCG 3′ (SEQ ID NO 11). The expression construct can further comprise a nucleotide sequence encoding a terminator such as a terminator having a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the expression construct comprises a nucleotide sequence having about 75% or more, 85% or more, 95% or more, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a sequence selected from SEQ ID NO: 21-SEQ ID NO: 26.

### III. Methods

One aspect of the present disclosure encompasses a method of treating levodopa-induced dyskinesia (LID) in a subject in need thereof. LIDs refer here to abnormal involuntary behaviors including dystonia, hyperkinesia, and/or stereotypies noted in the presence of DA agonists in parkinsonian subjects. In general, the subject has Parkinson's disease or is at risk of developing Parkinson's disease. Further, the subject can be undergoing DA agonist therapy, or is expected to undergo DA agonist therapy.

The subject can be a human, a livestock animal, a companion animal, a lab animal, or a zoological animal. In one aspect, the subject can be a rodent, e.g., a mouse, a rat, a guinea pig, etc. Non-limiting examples of suitable livestock animals can include pigs, cows, horses, goats, sheep, llamas and alpacas. Non-limiting examples of companion animals can include pets such as dogs, cats, rabbits, and birds. As used herein, a “zoological animal” refers to an animal that can be found in a zoo. Such animals can include non-human primates, large cats, wolves, and bears. Non-limiting examples of a laboratory animal can include rodents, canines, felines, and non-human primates. Non-limiting examples of rodents can include mice, rats, guinea pigs, etc. In some aspects, the subject is a human subject.

The subject can be, without limitation, a human, a non-human primate, a mouse, a rat, a guinea pig, or a dog. In some aspects, the subject is a human subject. The subject can be a premature newborn, a term newborn, a neonate, an infant, a toddler, a young child, a child, an adolescent, a pediatric patient, or a geriatric patient. In one aspect, the subject is an adult patient. In another aspect, the subject is an elderly patient. In another aspect, the subject is between about 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old.

The term “treating” includes prophylactic and therapeutic treatments. The terms prophylactic and therapeutic are art-recognized and include the administration of one or more systems to a subject. If the system is administered prior to a clinical manifestation of an unwanted symptom or condition (e.g., before a subject develops LIDs), then the treatment is prophylactic (i.e., it protects the host against developing the unwanted condition), whereas if the system is administered after manifestation of the unwanted condition (e.g., after a subject develops LIDs), the treatment is therapeutic (i.e., it is intended to diminish, ameliorate, or stabilize the existing unwanted condition or side effects thereof). Thus, treating relates to the administration of a system, such that at least one symptom of a condition is decreased or prevented from worsening in a subject or group of subjects relative to a subject or group of subjects who did not receive the system; and treating also relates to the administration of a system, such that the risk that a symptom will develop or worsen is diminished in a subject or group of subjects relative to a subject or group of subjects who did not receive the system.

In some aspects, the method prevents the induction of dyskinesia in a subject undergoing levodopa therapy or expected to undergo levodopa therapy. In other aspects, the method reduces dyskinesia in a subject undergoing levodopa therapy. In other aspects, the method eliminates dyskinesia in a subject undergoing levodopa therapy. In other aspects, the method reverses ore-existing dyskinesia in a subject undergoing levodopa therapy.

It will be appreciated by those skilled in the art that a combination of more than one system of the present disclosure can be used. It will also be appreciated by those skilled in the art that a system of the present disclosure can be used in combination with other therapeutic agents before, after, and/or during treatment with a system of the disclosure. Further, methods of the invention can be used in combination with standard treatments for a specific disorder.

The method comprises administering to the subject a therapeutically effective amount of a composition comprising an engineered genetic system for reducing the expression of a CaV1.3 protein in a target cell. The system comprises a CaV1.3 protein expression modification system; and a nucleic acid delivery system for delivering the expression system to the target cell. The system and vectors can be as described in Sections I and II above.

In some aspects, the systems are formulated into pharmaceutical systems and administered by injection. In some aspects, the pharmaceutical systems are injected directly into the striatum, the substantia nigra, or both. Direct injection can provide an additional level of specificity in providing tissue-specific reduction of expression of CaV1.3, to potentially limit off-target activity of the system. If desired, an Omaya reservoir can be placed within the surgical site to enable repeat administration of a system.

Formulations of pharmaceutically-acceptable excipients and carrier solutions are well known to those of skill in the art, as is the development of suitable dosing and treatment regimens for using the particular systems described herein in a variety of treatment regimens. These formulations can contain at least about 0.1% of the active ingredient or more, although the percentage of the active ingredient(s) may, of course, be varied and may conveniently be between about 1 or 2% and about 70% or 80% or more of the weight or volume of the total formulation. The amount of active ingredient in each therapeutically-useful system can be prepared is such a way that a suitable dosage will be obtained in any given unit dose of the compound. Factors such as solubility, bioavailability, biological half-life, route of administration, product shelf life, as well as other pharmacological considerations, can be contemplated by one skilled in the art of preparing such pharmaceutical formulations, and as such, a variety of dosages and treatment regimens may be desirable.

The pharmaceutical forms suitable for injectable use include sterile aqueous solutions or dispersions, and sterile powders for the extemporaneous preparation of sterile injectable solutions or dispersions. Dispersions may also be prepared in glycerol, liquid polyethylene glycols and mixtures thereof, and in oils. Under ordinary conditions of storage and use, these preparations contain a preservative to prevent the growth of microorganisms. In many cases the form is sterile and fluid to the extent that easy syringability exists. The preparations are stable under the conditions of manufacture and storage, and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it can include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, and the liquid diluent first rendered isotonic with sufficient saline or glucose. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage can occur depending on the condition of the host. The person responsible for administration can, in any event, determine the appropriate dose for the individual subject.

In some aspects, the delivery system of the engineered system is a rAAV vector. In some aspects, the engineered genetic system is an engineered vector-mediated system for reducing expression of a CaV1.3 protein in a target cell. The system comprises a nucleic acid expression construct encoding an interfering nucleic acid molecule having a nucleotide sequence that targets a sequence within a gene encoding the CaV1.3 protein to reduce the expression of the CaV1.3 protein. The system also comprises an rAAV vector encapsidating the nucleic acid construct for delivering the nucleic acid construct to the target cell. The expression construct can express an shRNA molecule comprising a nucleotide sequence complementary to a target sequence within a gene encoding the CaV1.3 protein, operably linked to a promoter. The vector-mediated system can provide continuous, high-potency, and target-selective mRNA-level silencing of striatal CaV1.3 channels. The shRNAs can be as described in Section 1(b), expression constructs can be as described in Section II, and rAAV vectors can be as described in Section 1(c).

In some aspects, the engineered genetic system is an rAAV vector for reducing the expression of a CaV1.3 protein in a target cell. The vector encapsidates a nucleic acid construct encoding a protein expression modification system engineered to reduce the expression of the CaV1.3 protein. The nucleic acid construct can be a nucleic acid expression construct for expressing an shRNA sequence targeting a sequence within a gene encoding the CaV1.3 protein, wherein the expression construct comprises a nucleotide sequence encoding the shRNA operably linked to a promoter.

Cell infection methods for infecting cells with the rAAVs are known. For instance, the cells can be infected with the rAAVs by contacting the cells with the rAAVs. For instance, the cells can be tissue culture cells, and they can be contacted with the rAAVs by adding the rAAVs to the cell culture. The cells can also be infected by delivering to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque).

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some aspects, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique can also be employed by the skilled artisan to administer the virions into the vasculature of an isolated limb to potentially enhance transduction into muscle cells or tissue. Moreover, in certain aspects, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, in addition to the rAAV and carrier(s), other conventional pharmaceutical ingredients can be included, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

In some aspects, rAAV compositions are formulated to reduce aggregation of AAV particles in the composition, particularly where high rAAV concentrations are present (e.g., ^{˜}1013 GC/ml or more). Methods for reducing aggregation of rAAVs are well known in the art and include, for example, addition of surfactants, pH adjustment, salt concentration adjustment, etc.

Formulation of pharmaceutically-acceptable excipients and carrier solutions is well known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared in such a way that a suitable dosage is obtained in any given unit dose of the compound. Factors such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

In certain aspects, it is desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some aspects, the cells are infected with the rAAVs by administering the rAAVs to a subject in a pharmaceutically-acceptable carrier to the subject in an amount and for a period of time sufficient to infect the cells. For instance, the rAAVs can be administered parenterally into the subject. When the cells are neural cells, including microglial cells, the rAAVs can be administered by injection into the striatum.

Pharmaceutical forms suitable for injectable use can include sterile aqueous solutions or dispersions and sterile powders for the extemporaneous preparation of sterile injectable solutions or dispersions. Dispersions may also be prepared in glycerol, liquid polyethylene glycols and mixtures thereof, and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed is known to those of skill in the art. For example, one dosage may be dissolved in 1 ml of isotonic NaCl solution and either added to 1000 ml of hypodermoclysis fluid or injected at the proposed site of infusion. Some variation in dosage may necessarily occur depending on the condition of the host.

Sterile injectable solutions can be prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient, plus any additional desired ingredient from a previously sterile-filtered solution thereof.

rAAVs can also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions are administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Precise delivery of a system into the striatum can be conducted using stereotactic microinjection techniques. For example, the subject being treated can be placed within a MRI-compatible stereotactic frame base and then imaged using high resolution MRI to determine the three-dimensional positioning for the particular injection. The MRI images are then transferred to a computer having the appropriate stereotactic software, and a number of images are used to determine a target site and trajectory for a microinjection. The software translates the trajectory into three-dimensional coordinates that are appropriate for the stereotactic frame. In the case of intracranial delivery, the skull is exposed, burr holes are drilled above the entry site, and the stereotactic apparatus positioned with the needle implanted at a predetermined depth. A pharmaceutical composition comprising a vector can then be microinjected at the selected target sites. In some aspects, the composition is injected by an osmotic pump or an infusion pump, such as a convection-enhanced delivery device. The spread of the vector from the site of injection can be a function of diffusion, which may be controlled by adjusting the concentration of the vector in the pharmaceutical composition.

Another aspect of the present disclosure encompasses a method of improving the response to levodopa or DA agonist in a subject in need thereof. Yet another aspect of the present disclosure encompasses a method of protecting DA neurons in the substantia nigra. An additional aspect of the present disclosure encompasses a method of slowing progression of Parkinson's disease by providing protection against death or dysfunction of substantia nigra dopamine neurons. The method comprise administering to the subject a therapeutically effective amount of a composition comprising an engineered genetic system of claim 1 for reducing the expression of a CaV1.3 protein in a target cell.

### IV. Kits

A further aspect of the present disclosure provides kits comprising one or more engineered system for reducing the expression of a CaV1.3 protein or one or more nucleic acid constructs encoding the engineered system, or a nucleic acid construct encoding a CaV1.3 protein expression modification system in a target cell. Engineered systems can be as described in Section I above, and nucleic acid constructs encoding the engineered system or the nucleic acid construct encoding a CaV1.3 protein expression modification system can be as described in Section II. Alternatively, the kit can comprise one or more cells comprising one or more engineered systems, one or more nucleic acid constructs encoding the engineered system, or the nucleic acid construct encoding a CaV1.3 protein expression modification system, or combinations thereof.

The kits can further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “subject” as used herein refers to a mammalian subject, including without limitation a human, a non-human primate, a rodent, a porcine, guinea pig, a canine, and a feline.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of autism spectrum disorder, “treat” and “treating” encompass alleviating, ameliorating, delaying the onset of, inhibiting the progression of, or reducing the severity of one or more symptoms associated with an autism spectrum disorder.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The term “a therapeutically effective amount” as used herein refers to an amount effective, at dosages and for periods of time necessary, to achieve a desired general protective effect, and the desired result with respect to the treatment of a disease or protection from a disease. For example, in the protection from a neurodegenerative disease, an agent (i.e., a compound or a composition) which protects from, decreases, prevents, delays, or suppresses or arrests any symptoms of the neurodegenerative disease would be effective. An effective amount of an agent is not required to cure a disease or condition but will provide a treatment for a disease or condition such that the onset of the disease or condition is delayed, hindered or prevented, or the disease or condition symptoms are ameliorated. The effective amount can be divided into one, two or more doses in a suitable form to be administered at one, two or more times throughout a designated time period. A therapeutically effective amount can be determined by the efficacy or potency of the particular composition, the disorder being treated, the duration or frequency of administration, the method of administration, and the size and condition of the subject, including that subject's particular treatment response. A therapeutically effective amount can be determined using methods known in the art, and can be determined experimentally, derived from therapeutically effective amounts determined in model animals such as the mouse, or a combination thereof. Additionally, the route of administration can be considered when determining the therapeutically effective amount. In determining therapeutically effective amounts, one skilled in the art can also consider the existence, nature, and extent of any adverse effects that accompany the administration of a particular compound in a particular subject.

As used herein, the terms “to cure,” “curative effect,” “treating,” “treatment,” and “to treat” can be used interchangeably and each can mean to protect from, alleviate, suppress, repress, eliminate, prevent or slow the appearance of symptoms, clinical signs, or underlying pathology of a condition or disorder on a temporary or permanent basis. Protection of cells or tissues involves administering an agent of the present invention to a subject prior to onset of a condition, even when development of the condition is not suspected. Treating a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment can reduce the risk of developing the condition and/or lessen its severity if the condition later develops. For instance, treatment of a microbial infection can reduce, ameliorate, or altogether eliminate the infection, or prevent it from worsening.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence can be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence can be modified such that an altered product is made.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide can be linked by phosphodiester, phosphothioate, phosphoramidite, phosphorodiamidate bonds, or combinations thereof.

The term “nucleotide” refers to deoxyribonucleotides or ribonucleotides. The nucleotides can be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog can be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence,” or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Genetic Silencing of Striatal CaV1.3 Prevents and Ameliorates Levodopa Dyskinesia

Symptomatic treatment of individuals with Parkinson's disease (PD) includes dopamine (DA) replacement therapies, with the DA precursor, levodopa, being the gold standard. Long-term levodopa therapy is associated with motor complications, including levodopa-induced dyskinesias (LID), that often negatively impact the quality of life of those afflicted. Although there are varying accounts of the occurrence, LIDs are estimated to occur in roughly 50% of PD patients after approximately 3-5 years of treatment, with the incidence escalating to approximately 90% after 10-15 years. Maintaining motor benefits of therapy while avoiding this often debilitating side effect remains an unmet clinical need.

Central to the biology of LIDs are changes in synaptic plasticity associated with striatal medium spiny neurons (MSNs), critical targets of convergent cortical glutamate and nigrostriatal DA input. MSNs, which constitute approximately 95% of striatal neurons, express both CaV1.3 and CaV1.2 voltage-gated L-type calcium channels. In PD and animal models of PD, striatal DA depletion results in loss of dendritic spines on MSNs, an aberrant feature accompanied by secondary loss of glutamate synapses from corticostriatal projections. Introduction of levodopa in this environment results in restoration of dendritic spines and reestablishment of glutamate input, but in an aberrant pattern of apparent “miswiring.”

Retraction of dendritic spines on MSNs associated with loss of striatal DA has been linked to dysregulation of intraspinous CaV1.3 channels. Blockade of these channels with CaV1.2/1.3 channel antagonists (i.e., nimodipine, isradipine) prevents spine retraction despite severe loss of DA in the parkinsonian striatum. As such, it has been hypothesized that preventing initial spine loss and associated synaptopathology in PD and models of PD may prevent abnormal rewiring of glutamate inputs and diminish liability for LID despite loss of DA. Indeed, it has been shown that CaV1.2/1.3 channel antagonists can prevent induction of LID produced by low-dose levodopa (6 mg/kg) and high-dose levodopa (12.5 mg/kg); however, this effect is partial and lost over time, and this paradigm is incapable of reversing established LID. The transient nature of the antidyskinetic effect of currently available CaV1.3 antagonists is speculated to be because of pharmacologic limitations of these drugs, including lack of specificity and potency for CaV1.3 channels.

The experiments performed in this example provide the first unequivocal evidence, devoid of pharmacological limitations, on the ability of CaV1.3 silencing to provide meaningful and lasting functional protection against LID. Described is a developed recombinant adeno-associated virus (rAAV)-mediated short hairpin RNA (shRNA) to provide continuous, high-potency, and target-selective mRNA-level silencing of striatal CaV1.3 channels. Presented herein are functional data demonstrating that delivery of rAAV-CaV1.3-shRNA to the DA-depleted striatum of unilaterally parkinsonian rats prior to the introduction of levodopa provides robust and lasting prevention of LIDs. Importantly, the data demonstrate that delivery of rAAV-CaV1.3-shRNA can in fact reverse LID in parkinsonian rats with established severe LID behavior.

### Methods, Experimental Subjects

All procedures were performed on adult male Sprague-Dawley (SD) rats (250 g; Envigo RMS Inc., Indianapolis, Ind.) in accordance with the Association for Assessment and Accreditation of Laboratory Animal Care International guidelines.

### Vector Design and Production

Portions of the CaV1.3 gene (Cacna1d) were cloned from rat cDNA and sequenced. shRNA sequences (CaV1.3 [5′-GAAGAGGCGCGGCCAAGAC-3′ (SEQ ID NO: 9)] or scrambled [5′-CAACAAGATGAAGAGCACC-3′ (SEQ ID NO: 10]) were designed using standard algorithms as described previously and compared against available rat genome data to ensure specificity. Particular attention was paid to ensure that the shRNA contained negligible overlap with CaV1.2. Then shRNA was cloned into a rAAV genome under control of the H1 promoter (AAV9.H1.shRNA-CaV1.3-Rat. SEQ ID NO: 27). The same genome contained green fluorescent protein (GFP) under control of the hybrid chicken b-actin/cytomegalovirus promoter as a transduction marker.

The rAAV was packaged into AAV9 capsids using triple transfection in HEK293T cells. Viral capsids were purified using an iodixanol step gradient and concentrated using buffer exchange. Virus titers were determined using dot-blot and normalized to 1×10¹³ vector genomes (vg)/m L.

### Dyskinesia Rating

LIDs refer here to abnormal involuntary behaviors including dystonia, hyperkinesia, and/or stereotypies noted in the presence of levodopa in parkinsonian rats. Injections were administered to allow assessment of LID behaviors for 1 minute every 30 minutes beginning 20 minutes after injection and continued up to 170 or 200 minutes. Rats were randomized and rated by the same blinded investigator throughout the experiment. Each rat was given a total LID severity score for each point assessed based on a rating system developed with a clinical movement disorders specialist (R.K.) as previously detailed.

### LID Prevention Studies

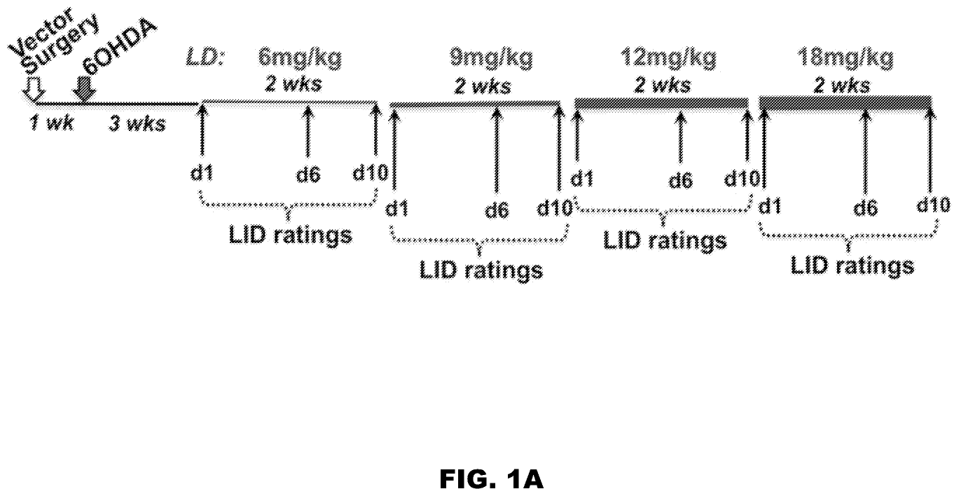

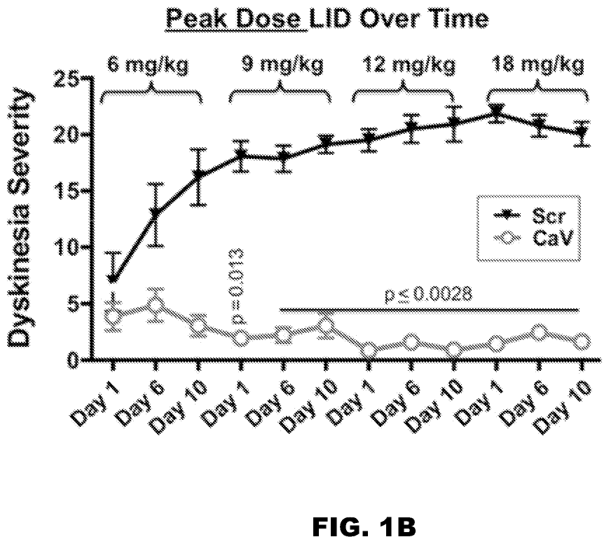

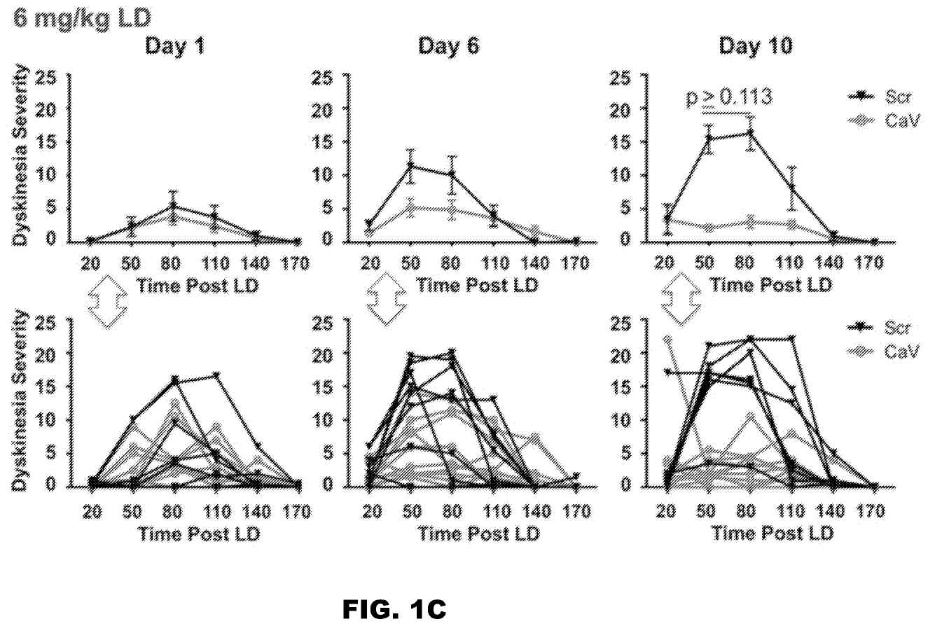

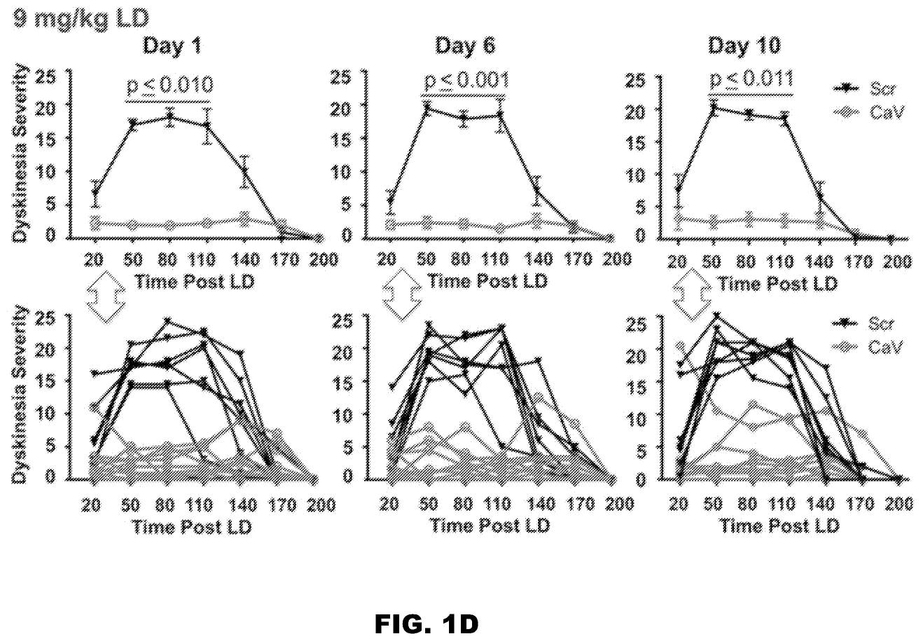

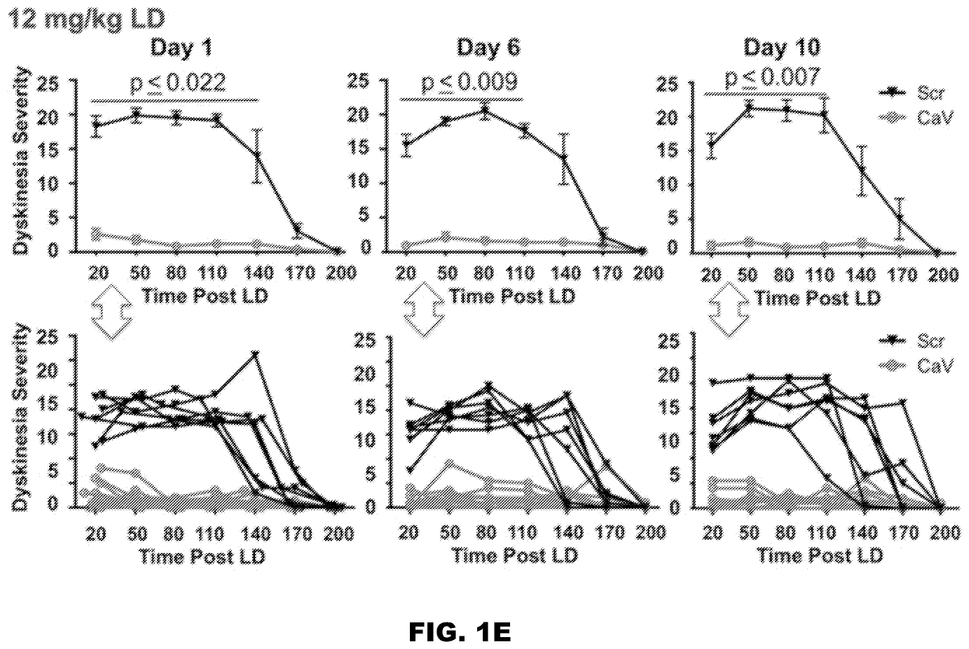

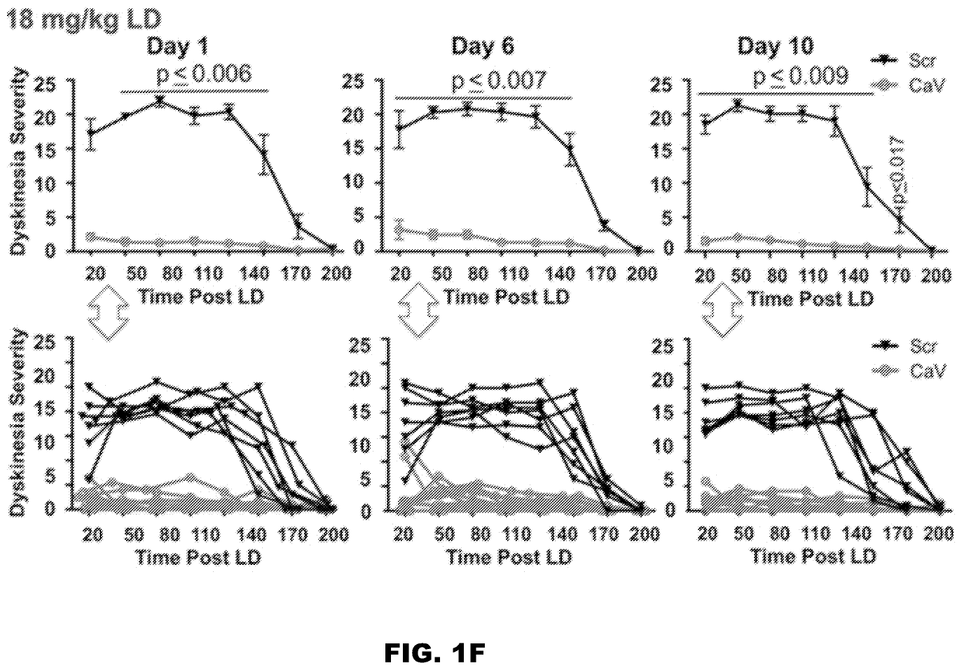

All stereotaxic surgeries (prevention and reversibility studies) were performed as previously detailed. To test the hypothesis that shRNA-mediated silencing of striatal CaV1.3 channels prior to levodopa would prevent the development and provide sustained amelioration of LIDs, rats received unilateral injections of either rAAV-CaV1.3-shRNA or the control rAAV-Scrambled (Scr)-shRNA (1.0×10¹³ vg/mL) into 2 dorsolateral sites within the left striatum (AP0.0, ML+3.0, DV-5.2; and AP+1.6, ML+2.7, DV-4.9). Vector surgery preceded by 1 week 6-hydroxydopamine (6-OHDA) neurotoxin surgery used to induce unilateral parkinsonism (FIG. 1A). Parkinsonism was induced using stereotaxic injection of 6-OHDA into substantia nigra (SN) and medial forebrain bundle per our usual protocol. Vector delivery prior to 6-OHDA was used to minimize spine loss associated with CaV1.3 dysregulation, which occurs secondary to striatal DA depletion. However, because AAV is retrogradely transported to nigral DA neurons in which CaV1.3 silencing could interfere with 6-OHDA-induced cell death, timing for these experiments was systematically worked out in pilot feasibility studies. Three weeks following 6-OHDA (4 weeks post-vector surgery), rats began receiving daily levodopa injections given at escalating doses ranging from low (6 mg/kg) to moderate (9 mg/kg) to high (12 mg/kg), to what is referred to here as “extreme” (18 mg/kg); see FIG. 1A. Each dose was given daily (Monday through Friday) for 2 weeks with a constant dose of carbidopa (12 mg/kg), a peripheral decarboxylase inhibitor. LID behaviors were rated on days 1, 6, and 10 of each dose.

### LID Reversibility Studies

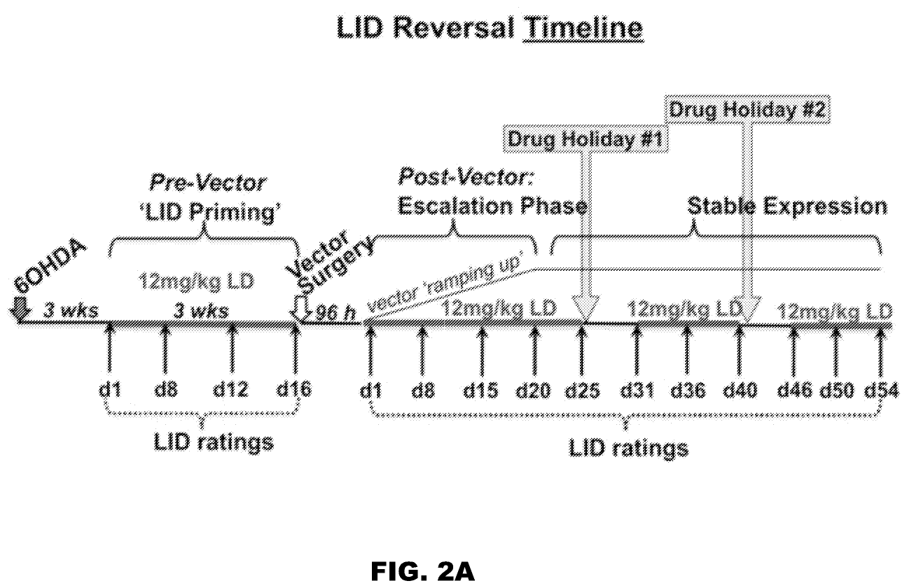

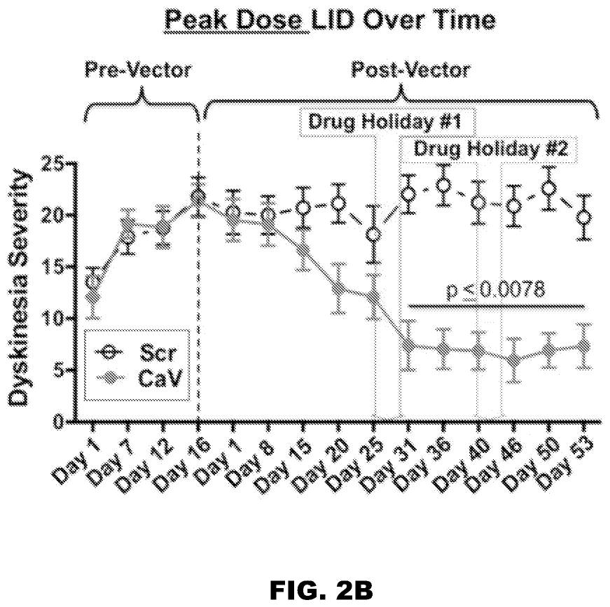

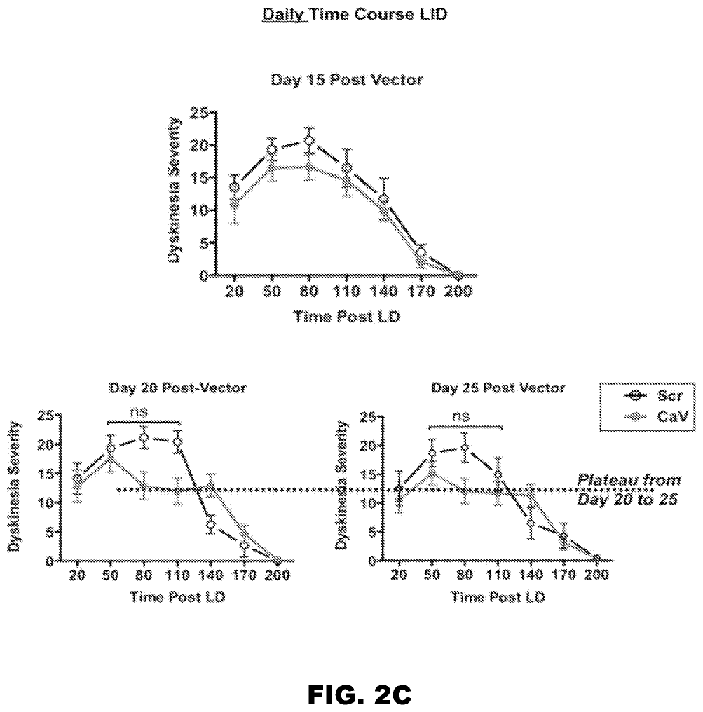

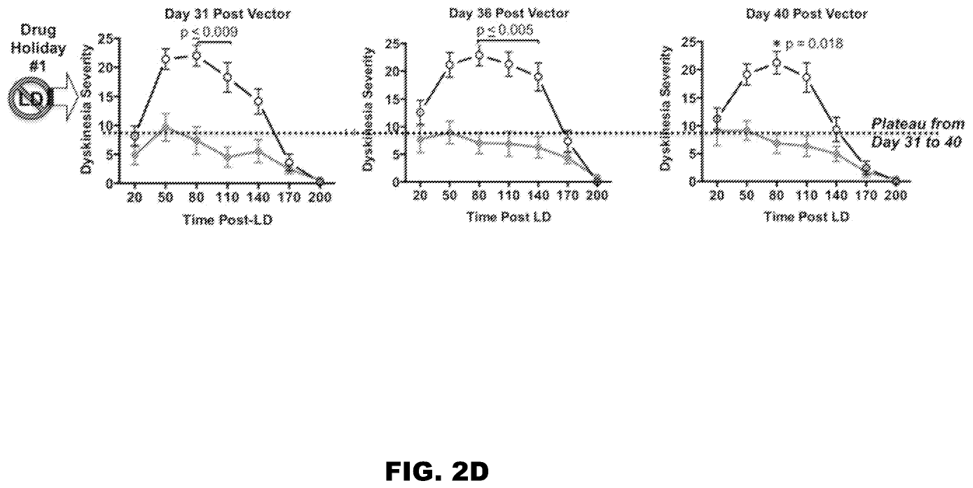

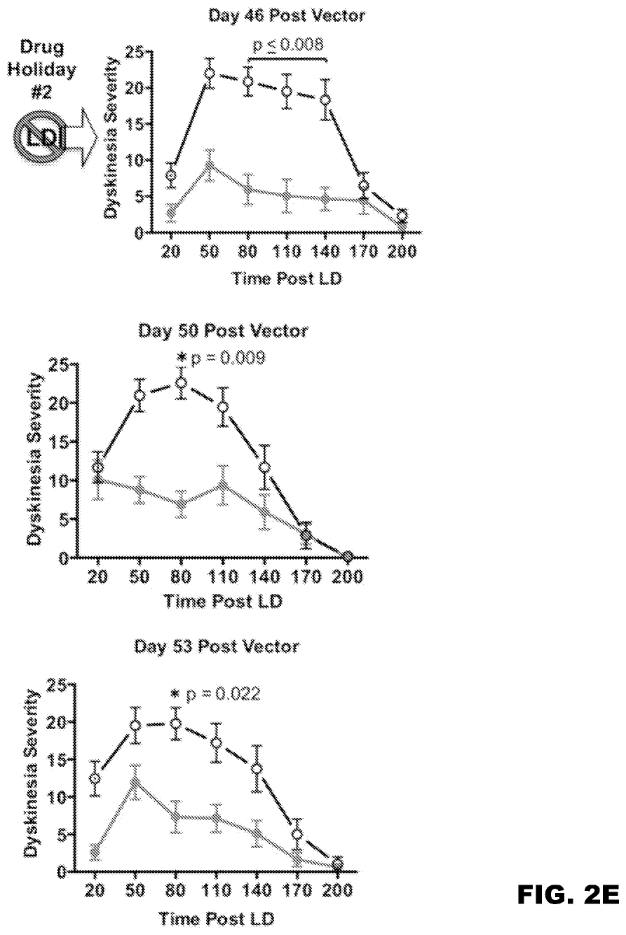

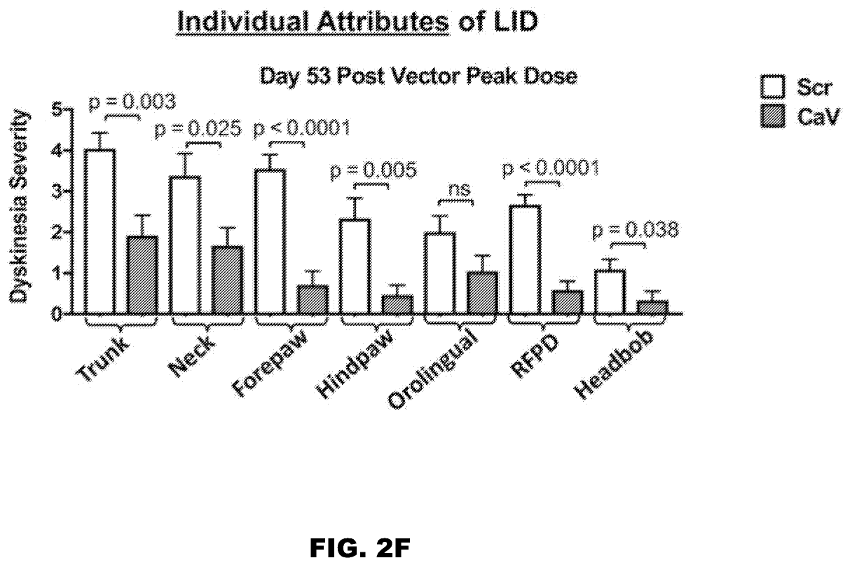

To test the hypothesis that in rats with established LID behaviors, ameliorating dysfunctional calcium signaling in an environment of established “miswiring” would lessen LIDs, rats were first rendered unilaterally parkinsonian. As depicted in FIG. 2A, 3 weeks after 6-OHDA rats began receiving daily high-dose levodopa (12 mg/kg, Monday through Friday; levodopa:carbidopa 1:1). After 3 weeks of treatment, all rats exhibiting stable, high levels of LIDs were assigned to either a rAAV-CaV1.3-shRNA or control rAAV-Scr-shRNA group. Groups were assigned to ensure the average peak-dose LID severity between groups was not different. Vector surgeries were identical to those for the LID prevention study.

### Cylinder Motor Test

A modified cylinder task was used to examine motor response in the absence of drug (prelevodopa [pre-LD]) and in response to low-dose levodopa (6 mg/kg; 12 mg/kg benserazide) in all rats in both prevention and reversibility studies. Rats were placed in a clear plexiglass cylinder (16 cm in diameter, 25 cm in height) and videotaped for 5 minutes prior to and again 50 mins post-levodopa injection. The number of 360° rotations and rears were quantified by a blinded investigator.

### Euthanasia

One day after the final LID behavioral rating, rats were administered a final dose of levodopa (12 mg/kg), LID-rated, and videotaped 50 minutes post-injection and subsequently euthanized per our usual protocol.

### TH, GFP, and NeuN Immunohistochemistry

All postmortem analyses were done by blinded investigators. To examine nigral lesion status, region of vector transduction, and lack of striatal neuron toxicity, individual series (1 in 6) of sections (40-μm thickness) were processed for tyrosine hydroxylase (TH; Millipore-AB152b rabbit anti-TH, 1:4000), GFP (Millipore-Ab290 rabbit anti-GFP, 1:20,000), or neuronal nuclei protein (NeuN; pan-neuronal marker of mature neurons; Millipore-Mab377, 1:1000) immunochemistry (IHC) per previously reported methods (TH, GFP, NeuN).

The degree of nigral DA neuron depletion in each animal was confirmed by total enumeration of TH-positive neurons. Nigral TH neuron loss of 95% in the lesioned hemisphere compared with the unlesioned hemisphere was used as a final inclusion criterion. This magnitude of SN DA neuron depletion is required in this model to produce reliable LIDs.

The striatal volume of vector transduction in GFP-immunostained sections was determined with the Cavalieri estimator using StereoInvestigator software (MicroBrightField, Williston, Vt.). Briefly, contours were defined for both the striatum and striatal region of GFP immunoreactivity. The outlines of each structure were traced at 1× in approximately 6 coronal sections along the entire rostral-caudal extent of the striatum.

Prior to undertaking the functional studies, a small cohort of rats received either rAAV-CaV1.3-shRNA (n=4) or rAAV-Scr-shRNA (n=4, as described above) to ensure the absence of non-specific shRNA or GFP-associated toxicity. The number of NeuN+ cells was quantified in the GFP+ region of striatum employing stereological techniques according to previously reported methods.

### CaV Knockdown Analyses

To determine degree and specificity of knockdown of the CaV1.3 transcript in the presence of our vectors, tissue from animals was employed in the LID prevention study. The relative abundance of CaV1.3 and CaV1.2 transcripts was quantified in the dorsolateral striatum using commercially available RNAscope in situ hybridization (ISH) (ACD, Newark, Calif.) probes generated against CaV1.3 (cacna1d, NM_017298.1, nucleotides 5401-6474) and CaV1.2 (Cacna1c, NM_012517.2, nucleotides 5183-6142). To estimate degree of CaV1.3 protein knockdown, dual-label CaV1.3 (Santa Cruz SC-515679, L-type-Ca²⁺CPα1D [CACNA1D] mouse anti-cav, 1:600) plus GFP IHC, and confocal microscopy was employed in a random subset of rats in which transcript was measured (n=4 rAAV-CaV1.3-shRNA, n=4 rAAV-Scr-shRNA).

For RNAscope ISH, 3 images in the GFP+ dorsolateral vector-injected or non-injected striatum were acquired on an OlympusBX51 light microscope at 20×. For dual-label IHC, 2 images in the vector-injected or non-injected striatum in the same general striatal regions as the ISH micrographs were acquired at 20× on a Nikon ElipseTi confocal using NIS Elements. The CaV immunoreactive ISH signal, which is sensitive enough to detect single transcripts, or CaV1.3 immunostaining analyses were performed using ImageJ software (NIH) using the threshold function. All microscope and camera settings were identical for all images within a given transcript or antibody. Data are represented as mean area or percent total area above the threshold. CaV1.3 transcript silencing is expressed as relative level of transcript in the rAAV-CaV-shRNA versus rAAV-Scr-shRNA striatum.

### Statistical Analyses

All LID behavioral data were analyzed with non-parametric statistics (Freidman [FM] or Kruskal-Wallis [H] with Dunn's multiple-comparisons test). Percent nigral lesion comparisons was analyzed with a nonparametric Mann-Whitney test. CaV1.3 mRNA expression in the rAAV-CaV-shRNA versus rAAV-Scr-shRNA striatum was compared using an unpaired, 2-tailed t test. Statistical evaluation of CaV1.2 mRNA expression in the injected and non-injected striata of rAAV-CaV-shRNA and rAAV-Scr-shRNA was done using 1-way ANOVA with Tukey's multiple-comparisons post hoc test. Nonparametric Spearman correlation test was used for correlation of LID severity with CaV1.3 levels. It was previously reported that in the outbred SD rat strain there is a small subset (15%-20%) of subjects that despite having an equal degree of striatal DA depletion and levodopa, treatment remains resistant to LID. These rats were characterized as LID negative and having a peak LID severity of 3.0. In the LID prevention study, 2 statistical outliers in the rAAV-Scr-shRNA group were identified, verified with the ROUT method (robust regression and outlier removal test; 3-step test automated within Prism; suggested Q coefficient detects outliers with false discovery rate <1%) and having a cumulative mean (across all 4 doses of levodopa) of 0.5±0.3 and 2.5±0.5; these 2 rats were omitted from final LID analyses. In addition, 1 rat in the AAV-CaV1.3-shRNA group in the LID prevention study was not adequately lesioned (ie, 21.6%) and was excluded from final behavioral analyses. Final subject numbers for the LID prevention study were: rAAV-CaV1.3-shRNA, n=10; rAAV-Scr-shRNA, n=7; for the LID reversibility study were: rAAV-CaV1.3-shRNA, n=11; rAAV-Scr-shRNA, n=12. All analyses were performed with Prism GraphPad. v7 for MacOSX.

### Results

Striatal rAAV-CaV1.3-shRNA Potently Prevents Induction of LID

Levodopa was administered using a dose-escalation paradigm beginning 4 weeks post-vector (3 weeks post-6OHDA; FIG. 1A). As demonstrated in FIG. 1B, rats receiving the control rAAV-Scr-shRNA displayed a typical escalation in LID severity over time that remained stable and severe with increasing doses of levodopa (Scr [black line]: day 1, 6 mg/kg versus day 6, 10-12 and 10-18 mg/kg; FM, 39.63; P=0.0002; Dunn's post hoc P 0.0326; FIG. 1B). In contrast, rats treated with rAAV-CaV1.3-shRNA showed significant suppression of the development of LIDs compared with rAAV-Scr-shRNA rats (red lines, Cay; FIGS. 1B-1F). This notable prevention of LID escalation continued to persist as a near-complete absence of dyskinetic behavior in the rAAV-CaV-shRNA rats, which showed a significant difference from control rAAV-Scr-shRNA rats beginning on day 1 of 9 mg/kg levodopa (H, 159.6; P<0.0001; Dunn's post hoc: Scr vs CaV, 50 minutes; P=0.0027; Scr vs CaV, 80 minutes; P=0.0010; Scr vs CaV, 110 minutes; P=0.0109). This potent anti-dyskinetic effect was noted in all rAAV-CaV-shRNA parkinsonian rats despite continuing elevation in dose of levodopa over 2 months (FIGS. 1C-1F, lower graphs illustrate uniform individual subject responses; detailed statistics provided for each day/dose in figure legends).

Quantification of TH-positive SN DA neuron loss revealed equivalent 95% depletion in both CaV and Scr groups (CaV, 98.6%±0.5%; Scr, 98.3%±0.5%; TH-positive cell loss compared with intact side; mean±SEM; P=0.7642 Mann-Whitney U test).

Striatal rAAV-CaV1.3-shRNA Partially Reverses Established, Severe LID

To examine whether striatal CaV1.3 silencing would impact expression of established LIDs, parkinsonian rats received chronic high-dose levodopa (12 mg/kg) for 3 weeks prior to receiving an intrastriatal injection of either rAAV-CaV1.3-shRNA or control rAAV-Scr-shRNA vector (FIG. 2A). Groups were balanced to ensure that the average peak-dose LID severity between the groups did not differ (Mann-Whitney, 2-tailed, P=0.6631; U=53.50; mean rAAV-CaV1.3, 22.8±0.7; rAAV-Scr, 23.2±1.3; median rAAV-CaV1.3, 23; rAAV-Scr, 24).

As depicted in FIGS. 2B-2E, rAAV-mediated CaV1.3 silencing in parkinsonian rats with established high-level LIDs can result in progressive and significant amelioration of existing LIDs. Specifically, the ability of CaV1.3 silencing to reverse peak-dose LIDs showed a non-significant trend of decreasing LID severity in the rAAV-CaV1.3-shRNA group (FIGS. 2B-2E) compared with the rAAV-Scr-shRNA group beginning on day 20 post-vector. This trend continued to be non-significant through day 25. As such, it was examined whether a short, 5-day withdrawal of levodopa (i.e., “drug holiday”), once popular for improving patient response to levodopa including potentially decreasing LIDs, would provide an environment in which vector-mediated reduction in aberrant calcium signaling might translate into reduction of LIDs.

As presented in FIGS. 2B-2E, on day 31 post-vector, the first day immediately following the 1-week levodopa-free drug holiday, LID severity in rAAV-CaV1.3-shRNA rats was significantly reduced compared with that seen in the control rAAV-Scr-shRNA group that predictably maintained a sustained high level of LID severity from the pre-vector through the entire post-vector time frame (day 31, F=135.7, P<0.0001; post hoc Dunn's test P=0.0009; FIG. 2B; F=97.2, P<0.0001; post hoc Dunn's test P 0.0094; FIG. 2C). The benefit from this first drug holiday for rAAV-CaV1.3 rats was sustained over the next 2 weeks (FIGS. 2B-2C, day 31 through day 40); however, there was no further diminution of LIDs with continued daily high-dose levodopa. Given this plateau of response, together with the apparent benefit of the first drug holiday, a second 1-week drug holiday was employed to determine whether further enhancement of LID reversal was possible. However, there was no further dampening of LID severity for the rAAV-CaV1.3 vector group after a second 1-week drug holiday when levodopa was maintained at this high dose of 12 mg/kg (day 40 vs day 46, P>0.999).

Quantification of TH-positive SN neuron loss in the lesioned hemisphere of all rats revealed nearly identical levels of unilateral lesioning between CaV and Scr groups (CaV, 99.1%±0.3%; Scr, 98.4%±0.6%; cell loss compared with the intact side, P=0.9394 Mann-Whitney).

Striatal CaV1.3 Silencing does not Interfere with Levodopa Motor Response

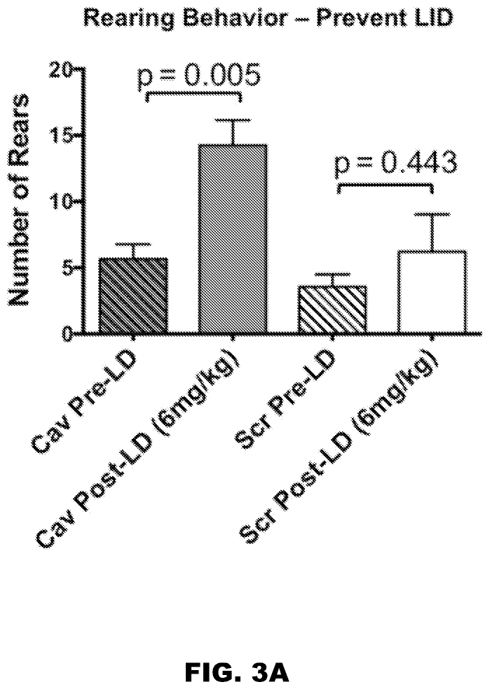

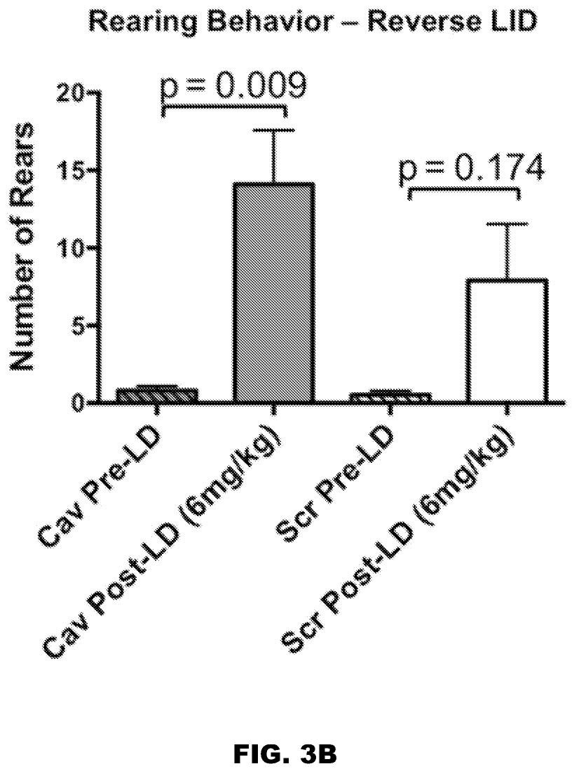

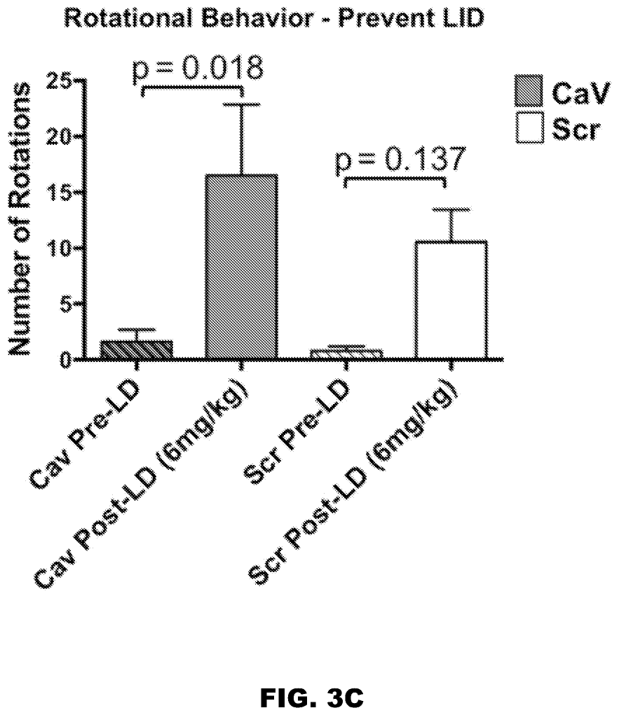

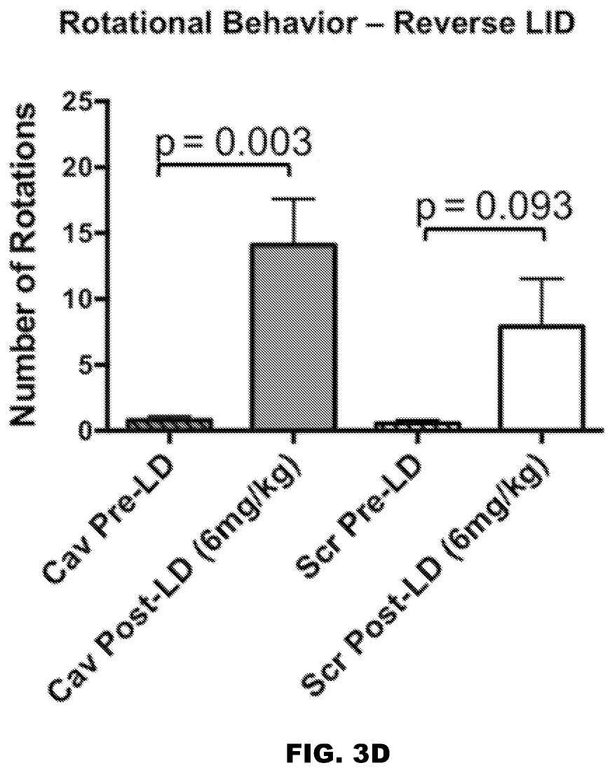

Quantification of exploratory rearing and contralateral rotational behavior using a modified cylinder test revealed no significant difference in either behavior under baseline drug-free (pre-LD) conditions (2-way ANOVA with post hoc Sidak's multiple-comparisons test; rearing, P=0.8503 for LID prevention, P=0.9999 for LID reversibility; rotations, P=0.9984 for LID prevention, P=0.9999 for LID reversibility). However, following low-dose levodopa (6 mg/kg), rAAV-CaV1.3-shRNA—but not control rAAV-Scr-shRNA-treated rats showed a significant increase in both exploratory rearing and rotational behavior compared with baseline levels (FIGS. 3A-3D) contains detailed statistics), suggesting that knockdown of CaV1.3 channels in the DA-depleted striatum may enhance motor response to levodopa. Additional evidence demonstrating that motor effects of levodopa were also readily apparent and not impaired by gene-level CaV1.3 silencing.

mRNA Knockdown, Specificity of Knockdown, and Transduction Spread

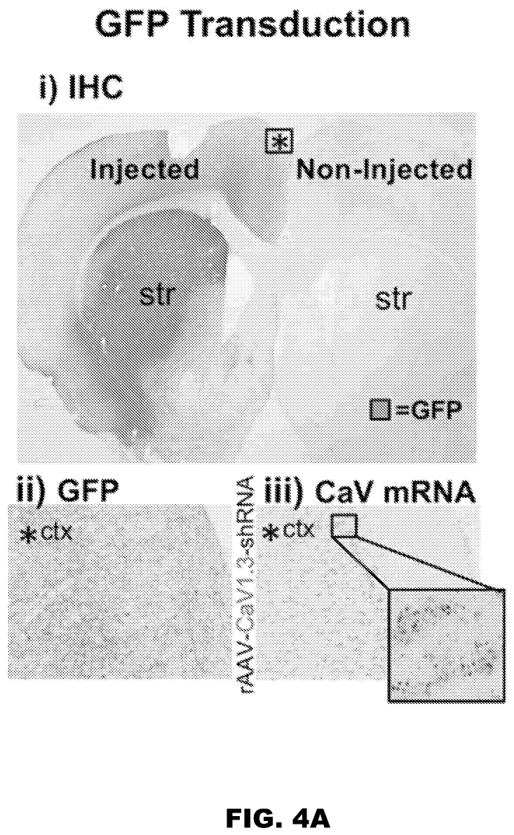

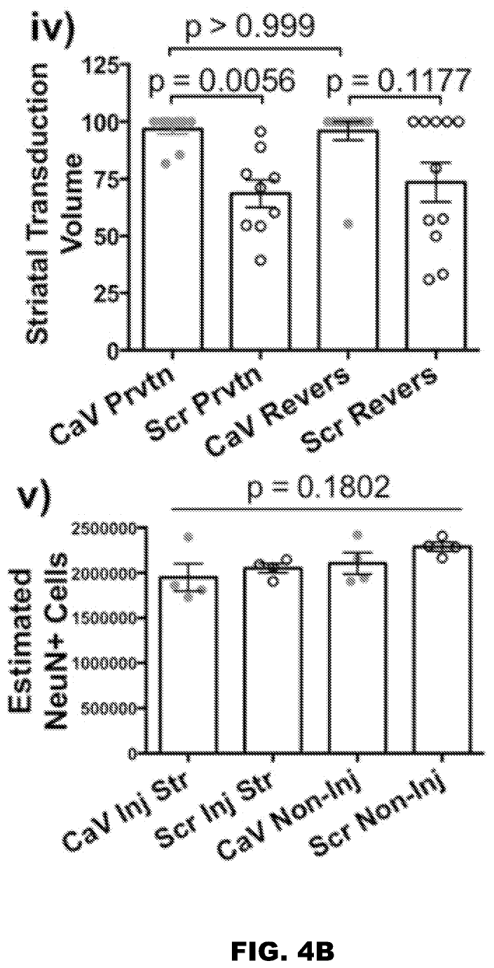

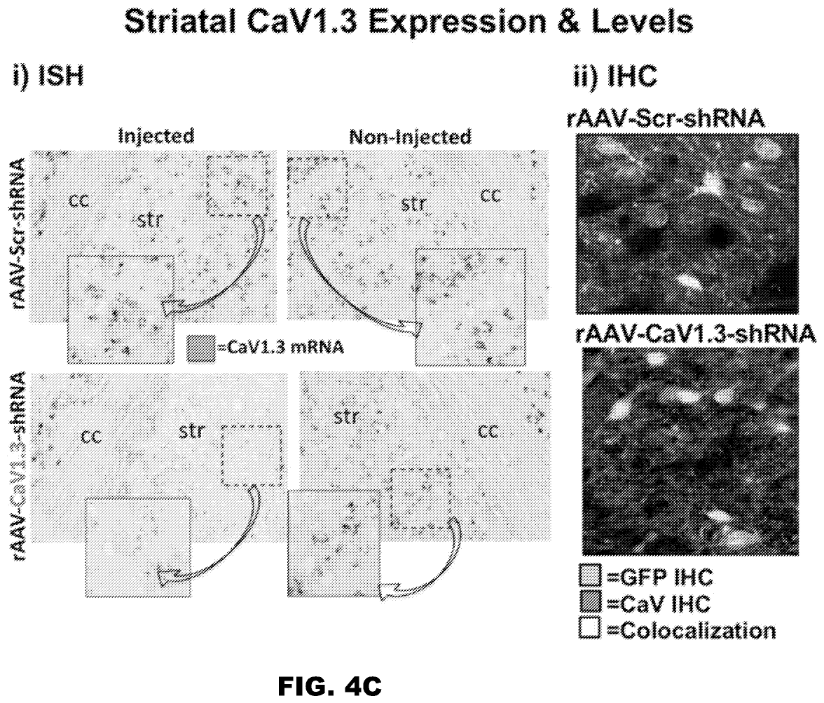

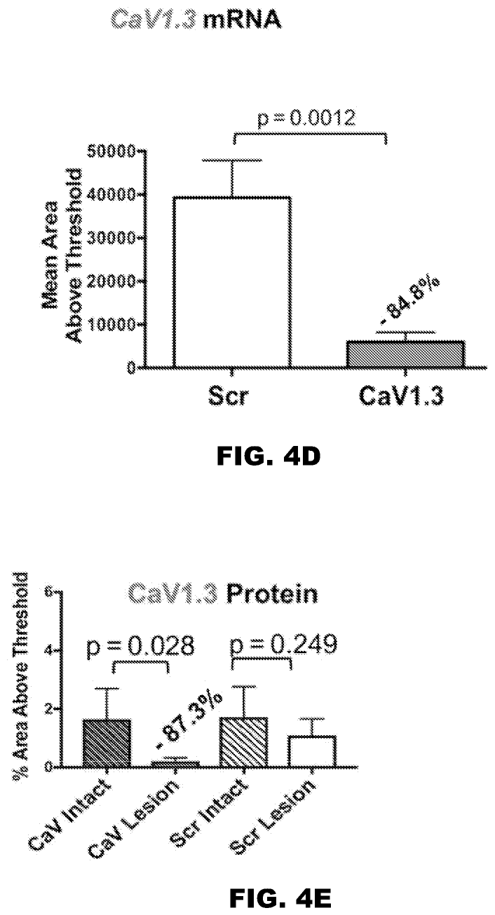

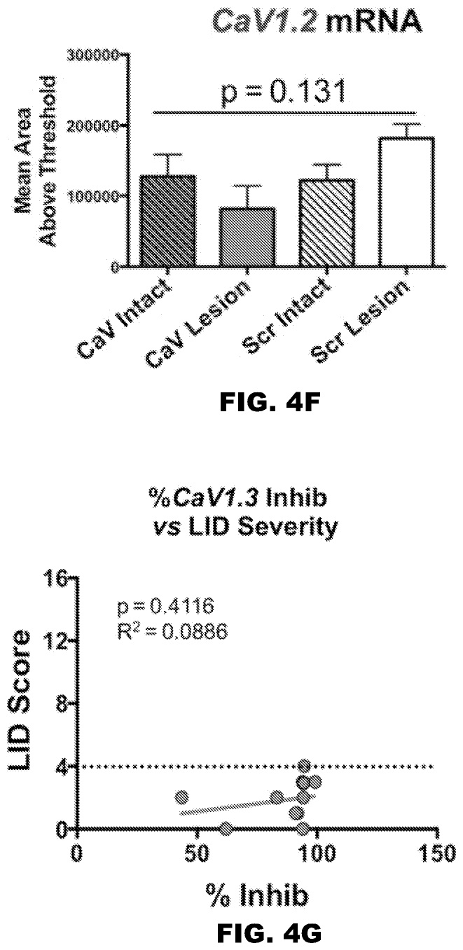

ISH analyses using probes generated against CaV1.3 (Cacna1d) and CaV1.2 (Cacna1c) mRNA revealed that the rAAV-CaV1.3-shRNA vector resulted in an average 84.77% reduction in CaV1.3 mRNA in the GFP-transduced region of the striatum compared with that seen with rAAV-Scr-shRNA (FIGS. 4A, 4B, and 4D-4F). A similar degree of CaV1.3 protein knockdown was found (87.3%; FIG. 4E). The range of mRNA silencing was 43.6% to 99.1% (FIG. 4G). However, there was no significant correlation of degree of CaV1.3 silencing with final LID severity scores (Spearman correlation, r=0.0886, 2-tailed P=0.4116), suggesting that even partial silencing of overactive CaV1.3 channels in the parkinsonian striatum is capable of completely preventing LID induction. Importantly, in contrast to an 84.77% knockdown of CaV1.3 mRNA, we detected no significant change in CaV1.2 mRNA (FIG. 4C iii), demonstrating the specificity of our genetic approach.

There was no difference in the volume of striatal transduction in rAAV-CaV1.3-shRNA rats between the LID prevention and LID reversibility studies, nor between these 2 studies in rats injected with rAAV-Scr-shRNA (Kruskal-Wallis, post hoc Dunn's multiple comparisons: P>0.999; FIG. 4E). Although not of functional significance, there was less volume of the striatum transduced from the inert control rAAV-Scr-shRNA vector compared with the rAAV-CaV1.3-shRNA in rats in the LID prevention study (P=0.0056; FIG. 4E). No evidence of toxicity related to the vectors was found, as demonstrated by equal numbers of NeuN-positive cells in intact versus vector-injected striatum regardless of the vector (ANOVA P=0.182, F=1.920; FIG. 4B).

As depicted in FIG. 4A, GFP expression was prominent in the target region of the striatum, but could also be seen as a result of retrograde transduction to striatal input areas including various cortical regions. In some but not all animals, sparse and highly variable patterns of GFP+ fibers were also noted in the globus pallidus, medial and/or lateral geniculate nuclei, and thalamic and/or hypothalamic regions. Despite the variable presence of extrastriatal GFP-immunoreactive fibers, little GFP was observed in cell bodies (e.g., FIG. 4A ii). Visual examination of CaV1.3 ISH in these extra-striatal regions confirmed that despite some transduction, there was no apparent reduction in cellular CaV1.3 mRNA levels (FIG. 4B). These findings are in agreement with the relatively low retrograde transduction efficacy seen with wild-type AAV capsids. Although involvement of CaV1.3 silencing in extra-striatal regions cannot definitively be ruled out as a contributing factor to the LID prevention/amelioration in the current studies, the highly variable nature of its expression in such regions, in the face of uniform LID reduction suggests that extra-striatal CaV1.3 silencing is not the principal mechanism of LID suppression.

### DISCUSSION

The current genetic-based studies, developed to provide high-potency striatal CaV1.3 channel silencing, were derived from a set of exploratory preclinical studies aimed at demonstrating the novel application of common US Food and Drug Administration-approved antihypertensive dihydropyridine (DHP) drugs for a new therapeutic application in PD, specifically prevention and/or reversal of LIDs. Indeed, although initial studies provided evidence that pharmacological antagonism of CaV1.3 channels could dampen LID in parkinsonian rats, the efficacy of these DHP drugs (i.e., isradipine and nimodipine) was partial and transient.

Using the approach of continuous, high-potency, target-specific genetic silencing of striatal CaV1.3 calcium channels with rAAV-CaV1.3-shRNA, it was demonstrated that mRNA-level silencing can provide potent, uniform, and long-term (>2 months) prevention of LIDs, even with extreme doses of daily levodopa. In addition, this approach was capable of reversing preexisting severe LIDs, which was not possible with pharmacological CaV silencing. Importantly, motor benefit from levodopa was maintained with the knockdown of CaV1.3. These findings provide some of the strongest preclinical data to date, demonstrating the amelioration of LIDs without compromise of motor benefit.

### LID Reversibility and Clinical Relevance

It is perhaps most compelling, and surprising, based on the presumed mechanism of LID prevention being the prevention of PD-associated spine changes, that the approach of CaV1.3 silencing employed in these studies could significantly reverse severe LID behavior in parkinsonian rats. In addition, our experimental design in these proof-of-principle studies included a short-term withdrawal of levodopa that coincided with a significant reduction of LIDs in rAAV-CaV1.3-shRNA rats when high-dose (12 mg/kg) treatment was reintroduced, which contrasted with maintenance of severe LIDs in rAAV-Scr-shRNA control rats. It is unclear whether levodopa withdrawal at this early point after vector surgery (i.e., days 26-30 post-vector) was necessary for this significant amelioration of LIDs. Accordingly, short-term drug withdrawal in the presence of CaV1.3 silencing may not enhance or be necessary for LID amelioration.

It is clear that there are adaptations in MSNs unrelated to dendritic spine density that may impact LID in the presence of CaV1.3 silencing. Specifically, these channels are linked directly to signaling cascades that not only impact, for example, long-term alterations in synaptic strength and short-term dendritic excitability, but also impact synaptic function through selective signaling to the nucleus altering transcriptional activity. The loss of DA tone (specifically D2 receptor tone) after DA depletion disinhibits CaV1.3 channels, leading to structural (e.g., spine retraction, synaptic pruning) and functional adaptations. Accordingly, silencing dysfunctional CaV1.3 channel activity in the parkinsonian striatum may ameliorate LIDs through functional adaptations distinct from altered dendritic spine density per se.

The ability to reverse LIDs holds immediate clinical relevance. Indeed, it could be anticipated that if gene therapy were undertaken when LID severity and/or levodopa dose were low (e.g., early in the disease), complete reversal and stable suppression of LIDs could be achieved. This idea is supported by the LID prevention studies demonstrating that once expression of the CaV1.3 channel and LIDs are maximally suppressed, the underlying mechanism(s) allowing for this near-complete amelioration is maintained despite the escalation of levodopa doses to very high levels.

### Improved Response to Levodopa

The motor behavior findings appear to suggest that knockdown of CaV1.3 channels in the DA-depleted striatum does not impede and may benefit motor response to levodopa. Indeed, when a “sub-therapeutic” dose of levodopa was administered to our severely parkinsonian rats, only those with CaV1.3 silencing showed a significant motor response.

### Example 2. Impact of Reduced Expression of CaV1.3 on LID in Aged Rats and Cynomolgus Macaques

Acknowledging the strong link between aging and Parkinson's disease, the ability of the viral vectors to knockdown expression of striatal CaV1.3 calcium channels was assessed and the impact of this LID in aged (15-19 months old (mo)) Fisher 344 (F344) rats and 20-24 year old cynomolgus macaques (Macaca fasicularis), which based on the average life expectancy of this species should equate to an approximately 60 year old human.

### Rat Data

To test the hypothesis that aged, parkinsonian subjects (i.e., rats) will maintain therapeutic benefit of striatal CaV1.3 calcium channel silencing against levodopa-induced dyskinesias (LID), male F344 rats (15 mo at time of lesion; 19 mo at time of sacrifice) were rendered parkinsonian using routine methods. Three weeks after 6OHDA-induction of experimental parkinsonism and prior to vector injection, animals were rendered mildly/moderately dyskinetic by administering a ‘low dose’ (3 mg/kg) of levodopa M, W, Fr for 20 days. Parkinsonian rats displaying stable mild/moderate LID then received an intrastriatal injection of either rAAV-CaV1.3-shRNA or the scrambled (Scr) control vector rAAV-Scr-shRNA. Levodopa was withdrawn the day of surgery and for a total of 96 hours after surgery to allow the rats to recuperate from surgery.

Once levodopa treatment was re-initiated, a dose escalation paradigm (3 mg/kg, 6 mg/kg, 12 mg/kg) was used to specifically test the hypothesis that vectored striatal CaV1.3 silencing will allow for reversal of mild-to-moderate LID, and prevent escalation of LID severity with increasing doses of levodopa.

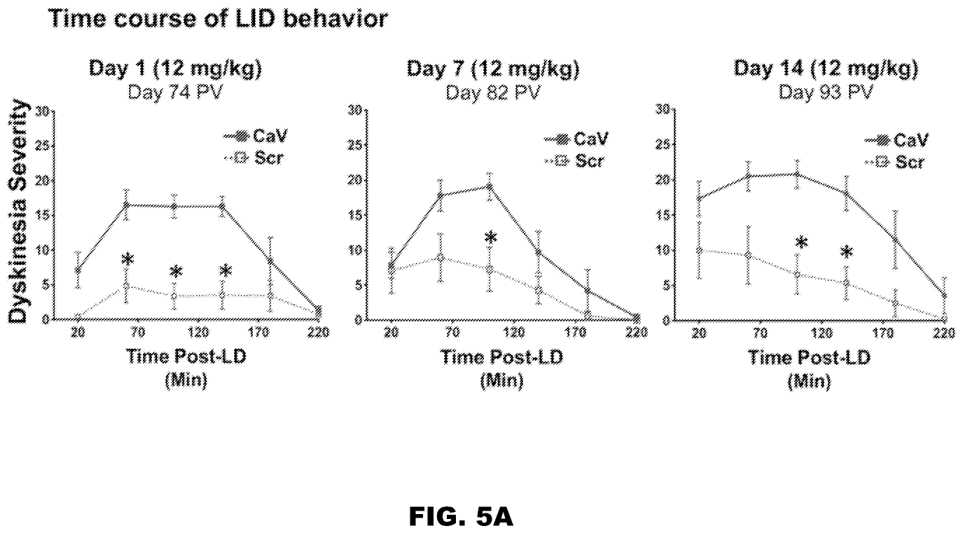

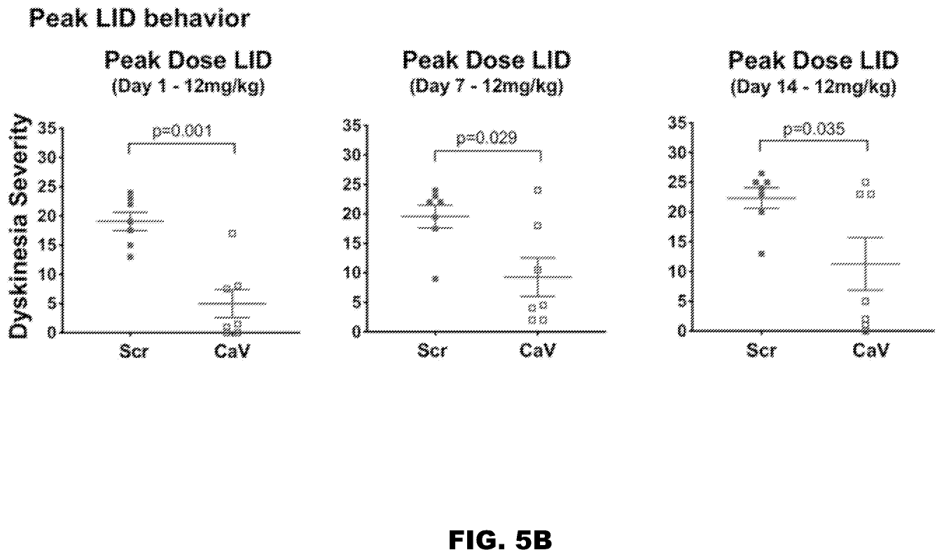

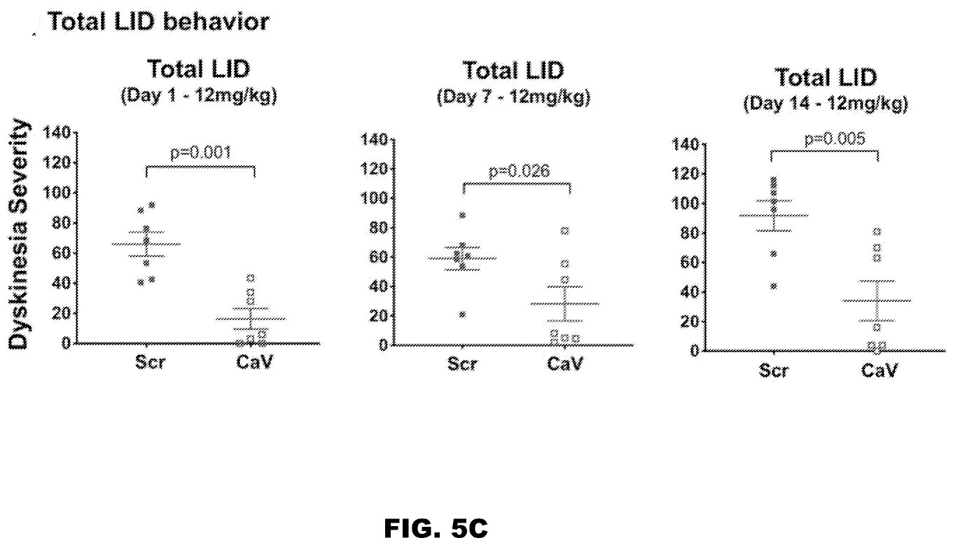

FIGS. 5A-5C show the final phase of levodopa treatment where the dose of levodopa was escalated to 12 mg/kg. This ‘high dose’ was administered over the final two weeks of the experiment and clearly demonstrates that, consistent with the hypothesis, there is a significant preservation of reduced LID severity in aged parkinsonian rats receiving rAAV-CaV-shRNA compared to those receiving rAAV-Scr-shRNA.

### Non-Human Primate Data

The findings in the rat model of PD demonstrating that gene silencing of CaV1.3 can prevent as well as reverse severe LID induced are highly encouraging, were also validated in other models of PD. Since the non-human primate model is the gold standard for preclinical testing, the anti-dyskinetic utility of rAAV-CaV1.3-shRNA in parkinsonian cynomolgus macaques (Macaca fasicularis) was examined. Primate shRNAs were designed using the same parameters as for the rodent version, albeit focused on sequences also covering the human gene.

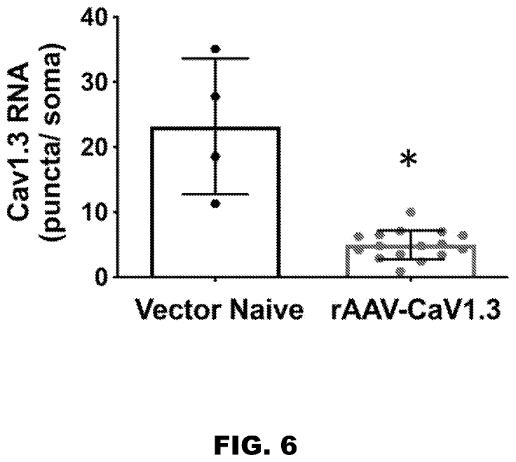

As shown in FIG. 6, there is an approximately 79% reduction in the level of CaV1.3 RNA in the striata of monkeys receiving an intra-striatal injection of the primate shRNA vector (rAAV-CaV1.3-shRNA) compared to vector naïve striata. The viral genome contained green fluorescent protein (GFP) as a marker of transduction. RNAscope® in situ hybridization was used to examine CaV1.3 mRNA levels in striatal GFP-positive neurons. Levels of CaV1.3 RNA were quantified using a computer generated Imaris®-3D reconstruction of confocal z-stack of striatal neurons dual labeled for GFP protein and CaV1.3 mRNA. Two fields of view (FOV) were taken from each of two control monkeys in the vector naïve striatum and in four monkeys that received rAAV-CaV1.3-shRNA vector.

### Example 3. Capacity to REVERSE LID in the NHP

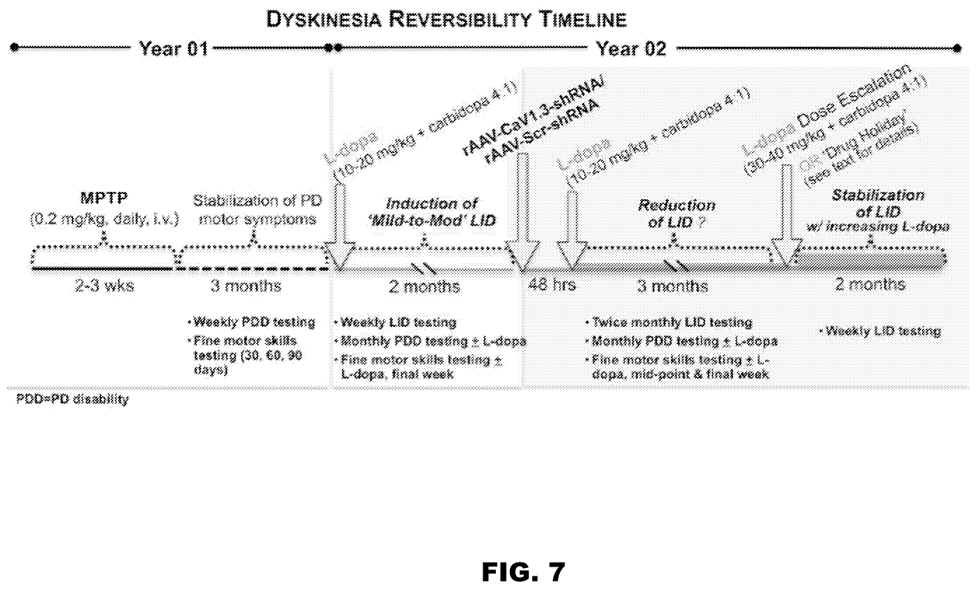

For these studies, captive bred monkeys (Macaca fasciculitis) are rendered bilaterally parkinsonian with daily MPTP injections until parkinsonian motor signs appear (˜2-3 weeks; FIG. 7 TIMELINE). Three to four days after beginning MPTP, parkinsonian motor signs are evaluated with a weekly ‘Parkinson's disease disability’ (PDD) test, which contains extensive evaluation of range of movement, bradykinesia, posture, and tremor, and once monthly fine motor skill testing as previously described. Following the stabilization of PD motor impairments (˜3-4 months), monkeys begin receiving daily levodopa treatment by oral gavage. Individual doses are titrated for the induction of mild-to-moderate LID severity (score range of 5-10) and reversal of PD motor symptoms, which are defined as the 100% dose. Animals are treated daily with their 100% dose of levodopa until stable expression of reproducible mild-to-moderate dyskinesia are observed, after which they receive levodopa every other day. LID, PDD and fine motor dexterity testing are video recorded in a custom video cage with a clear plexiglass front to allow unhindered viewing. Motor behaviors are recorded for 10 mins OFF and ON levodopa (ON=60 and 120 mins levodopa). LID is recorded at 4 time points post-levodopa (60, 120, 180, 240 mins) for 5 minutes per time point, and over a total period of 2 hours.

Once stable mild-to-moderate LID are established in the initial ‘LID induction phase’ (FIG. 7 TIMELINE), monkeys are assigned to one of the two treatment groups in a manner that ensures equal distribution of LID severity between groups. LID is evaluated within and between subjects. Each monkey receives either the rAAV-shRNA or control rAAV vector, generated specifically for Macaca fascicularis, stereotaxically injected bilaterally into the putamen, the motor region of the striatum in primates and the region showing elevated molecular markers of LID. Each monkey first receives a T1 weighted 3 Tesla MRI scan. Three targets aligned rostrocaudally and equispaced throughout the entire putamen are identified. Using sterile technique and isofluorane anesthesia, a midline incision is made, and with the guidance of the Stealth Navigation system, three burr holes are made bilaterally over the intended targets. Vector is injected into the rostral two sites and the caudal site. Injections are made at a rate of 1 ul/min and the needle is left in situ for an additional 5 min to allow the vector to diffuse from the needle tip. Monkeys are given 48-96 hours to recover from surgery prior to resuming levodopa treatment. The individual 100% dose of levodopa for each monkey is continued for an estimated 3 months, which will allow for evaluation of dyskinesias severity as the CaV1.3 shRNA expression increases and mRNA/protein levels diminish over time. Reversal of LID is noted in the rAAV-shRNA monkeys. The dose of levodopa is doubled from the 100% dose (estimated range 30-40 mg/kg) for the final 2 months to determine stability of antidyskinetic efficacy (FIG. 7 TIMELINE).

NHPs are sacrificed within 2-3 hours after the last levodopa dose (with final LID rating the day prior to sacrifice) in a manner that is compatible with light and electron microscopic histological analyses per usual protocol. Perfusion and postmortem endpoints, and analyses are as previously performed. However, in addition to ultrastructural assessment of corticostriatal (VGlut1), thalamostriatal (VGlut2) glutamate terminals using immunoelectron microscopy per our established protocol is examined.

There is near complete reversal of the mild-to-moderate LID in the monkeys that receive rAAV-shRNA compared to those injected with intraputamenal rAAV-shRNA. Further, the mild-to-moderate LID in these monkeys does NOT require a drug holiday for near complete reversal in the presence of rAAV-CaV1.3 vector. The rAAV-Scr monkeys demonstrate a mild but significant escalation of LID severity over the three-month post-vector timeframe despite the dose of levodopa remaining constant (i.e., the ‘100% dose’), a phenomenon typically noted with continuous levodopa. There is a significant reversal of LID with CaV1.3 silencing when the dose of levodopa is escalated from the 100% dose (10-20 mg/kg) to a high dose (twice the 100% dose for each monkey). Reversal of LID is stable. Similar to rats, there is NO interference of CaV1.3 silencing/normalization on motor benefit from levodopa in the rAAV-CaV1.3 vector monkeys, suggesting that this therapeutic approach has potential to transform the treatment of individuals with PD by allowing maintenance of motor benefit of levodopa in the absence of the debilitating LID side-effect. Overall, there is a linear relationship of LID expression levels with striatal mushroom spine density (Golgi) and VGlut1+ synapse density onto spines (EM) and striatal FosB and Nurr1 mRNA and protein, but not with striatal 5HT terminal density in both Aim 2 studies. In addition to VGlut1, VGlut2 immunoEM is included to allow the examination of remodeling of corticostriatal (VGlut1) and thalamostriatal (VGlut2) glutamatergic synapses in the NHP striatum in association with LID. As recently reviewed, while significant advances have been made in this field over the past several decades, many controversial issues remain and the specificity of the reorganization of synaptic connectivity in associated with striatal DA loss and LID remains poorly understood. In non-human primates, it has been shown that chronically MPTP-treated monkeys display a significant loss of thalamic neurons, specifically centromedian (CM) and parafascicular (Pf) nuclei, which is similar to that seen in PD patients. This neuronal loss impacts the relative abundance of VGlut2-positive terminals in the striatum.

### Example 4. Capacity to PREVENT LID in the NHP

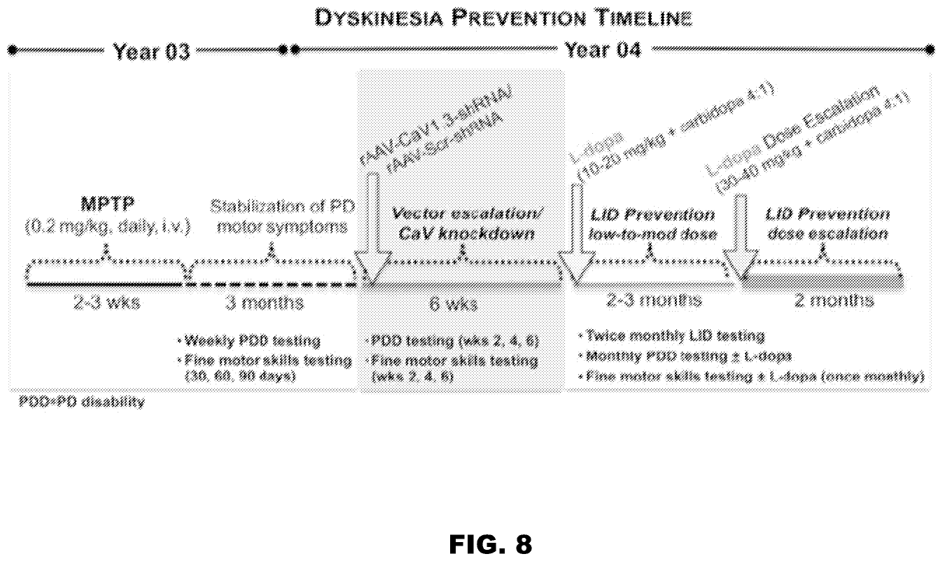

To most completely understand the clinical prospects and limitations of gene silencing of CaV1.3 in amelioration of LID, the capacity of CaV1.3 silencing in a prevention scenario in the NHP model is examined. For these LID prevention studies, monkeys are rendered unilaterally parkinsonian with daily MPTP injections until parkinsonian motor signs appear (˜2-3 weeks; FIG. 8 TIMELINE) and as detailed above in Example 3. Following the stabilization of PD motor impairments (˜3-4 months), six monkeys are assigned to one of the two treatment groups in a manner that ensures equal distribution of PDD severity between groups. Each monkey receives either the rAAV-shRNA or control rAAV vector, generated specifically for Macaca fascicularis, stereotaxically injected bilaterally into the putamen as described above. Six weeks after vector injection, a time of stable CaV1.3 mRNA and protein silencing, all monkeys begin treatment with daily (M-Fr) levodopa. The initial ‘low-to-moderate’ dose of levodopa (FIG. 8) is determined as the average of 100% dose from Example 3 (i.e., average dose used for the Induction of mild-to-moderate LID′). This dose is given daily for the first 2-3 weeks and then reduced to every-other-day as is standard for chronic administration in NHPs. If LID are significantly suppressed in the rAAV-Cav1.3-shRNA monkeys compared to the rAAV-Scr-shRNA, the dose of levodopa is doubled and continued for another 2 months (FIG. 8); if not, the same dose will be continued for final 2 months.

There is complete prevention of LID induction with the initial mild-to-moderate dose (10-20 mg/kg) of levodopa in the monkeys that receive rAAV-shRNA compared to those injected with control rAAV. Motor benefit of levodopa is NOT compromised and is equivalent between rAAV-shRNA and control rAAV monkeys. Escalation of the levodopa dose does NOT result in induction or escalation of LID in the rAAV-shRNA monkeys, but the rAAV-Scr-shRNA monkeys show a predictable and dramatic escalation of LID severity.

### Example 5. Neuroprotection

Selective knockdown of CaV1.3 expression in striatal neurons using shRNA (AAV9.H1.shRNA-CaV1.3) in rats rendered hem i-parkinsonian by delivery of 6-OHDA provides protection from, and reversal of, levodopa-induced dyskinesias (LIDs). This strategy also has important implications for protecting surviving dopamine (DA) neurons in the substantia nigra of individuals with Parkinson's disease. The rationale for targeting CaV1.3 channels in substantia nigra neurons comes from the fact that there is chronic elevation of intracellular calcium (Ca2+) in these neurons as a consequence of their autonomous pacemaking activity that is mediated by Cav1.3 L-type Ca2+ channels. Elevated intracellular Ca2+ is thought to result in physiological stress, rendering these neurons susceptible to toxic insults. Support for this rationale is that the pan-CaV1 channel antagonists isradipine and nimodipine have been shown to reduce dopaminergic neuron death in 1-methyl-4-phenyl-1,2,3,6-tetrahydropyridine (MPTP) and 6-hydroxydopamine (6-OHDA) lesioned rodents. However, the neuroprotective efficacy of these pan-CaV1 channel antagonists is partially due to partial target engagement, limited by off-target side-effects of higher doses. Target-specific inhibition of CaV1.3 expression in the substantia nigra using the compositions and methods described herein provides superior protection against PD-related insults in a manner not currently possible with pharmacological agents.

### REFERENCES

1. Huot, P., et al., The pharmacology of L-DOPA-induced dyskinesia in Parkinson's disease. Pharmacol Rev, 2013. 65(1): p. 171-222.
2. Hauser, R. A., et al., ADS-5102 (Amantadine) Extended-Release Capsules for Levodopa-Induced Dyskinesia in Parkinson's Disease (EASE LID 2 Study): Interim Results of an Open-Label Safety Study. J Parkinsons Dis, 2017. 7(3): p. 511-522.
3. Schuster, S., et al., Antagonizing L-type Ca2+ channel reduces development of abnormal involuntary movement in the rat model of L-3,4-dihydroxyphenylalanine-induced dyskinesia. Biol Psychiatry, 2009. 65(6): p. 518-26.
4. Soderstrom, K. E., et al., Impact of dendritic spine preservation in medium spiny neurons on dopamine graft efficacy and the expression of dyskinesias in parkinsonian rats. Eur J Neurosci, 2010. 31(3): p. 478-90.
5. Day, M., et al., Selective elimination of glutamatergic synapses on striatopallidal neurons in Parkinson disease models. Nat Neurosci, 2006. 9(2): p. 251-9.
6. Fieblinger, T. and M. A. Cenci, Zooming in on the small: the plasticity of striatal dendritic spines in L-DOPA-induced dyskinesia. Mov Disord, 2015. 30(4): p. 484-93.
7. Fieblinger, T., et al., Cell type-specific plasticity of striatal projection neurons in parkinsonism and L-DOPA-induced dyskinesia. Nat Commun, 2014. 5: p. 5316.
8. Ingham, C. A., S. H. Hood, and G. W. Arbuthnott, Spine density on neostriatal neurones changes with 6-hydroxydopamine lesions and with age. Brain Res, 1989. 503(2): p. 334-8.
9. McNeill, T. H., et al., Atrophy of medium spiny I striatal dendrites in advanced Parkinson's disease. Brain Res, 1988. 455(1): p. 148-52.
10. Nishijima, H., et al., Morphologic changes of dendritic spines of striatal neurons in the levodopa-induced dyskinesia model. Mov Disord, 2014. 29(3): p. 336-43.
11. Zhang, Y., et al., Aberrant restoration of spines and their synapses in L-DOPA-induced dyskinesia: involvement of corticostriatal but not thalamostriatal synapses. J Neurosci, 2013. 33(28): p. 11655-67.
12. Ingham, C. A., et al., Plasticity of synapses in the rat neostriatum after unilateral lesion of the nigrostriatal dopaminergic pathway. J Neurosci, 1998. 18(12): p. 4732-43.
13. Bastide, M. F., et al., Pathophysiology of L-dopa-induced motor and non-motor complications in Parkinson's disease. Prog Neurobiol, 2015. 132: p. 96-168.
14. Suarez, L. M., et al., L-DOPA treatment selectively restores spine density in dopamine receptor D2-expressing projection neurons in dyskinetic mice. Biol Psychiatry, 2014. 75(9): p. 711-22.
15. Kang, S., et al., Structure-activity relationship of N,N′-disubstituted pyrimidinetriones as Ca(V)1.3 calcium channel-selective antagonists for Parkinson's disease. J Med Chem, 2013. 56(11): p. 4786-97.
16. Xu, W. and D. Lipscombe, Neuronal Ca(V)1.3alpha(1) L-type channels activate at relatively hyperpolarized membrane potentials and are incompletely inhibited by dihydropyridines. J Neurosci, 2001. 21(16): p. 5944-51.
17. Kang, S., et al., CaV1.3-selective L-type calcium channel antagonists as potential new therapeutics for Parkinson's disease. Nat Commun, 2012. 3: p. 1146.
18. Rees, K., et al., Anti-hypertensive drugs as disease-modifying agents for Parkinson's disease: evidence from observational studies and clinical trials. Cochrane Database Syst Rev, 2011(11): p. CD008535.
19. Biglan, K. M., et al., A novel design of a Phase III trial of isradipine in early Parkinson disease (STEADY-PD III). Ann Clin Transl Neurol, 2017. 4(6): p. 360-368.
20. Zidanic, M. and P. A. Fuchs, Kinetic analysis of barium currents in chick cochlear hair cells. Biophys J, 1995. 68(4): p. 1323-36.
21. Collier, T. J., et al., Interrogating the aged striatum: robust survival of grafted dopamine neurons in aging rats produces inferior behavioral recovery and evidence of impaired integration. Neurobiol Dis, 2015. 77: p. 191-203.
22. Ilijic, E., J. N. Guzman, and D. J. Surmeier, The L-type channel antagonist isradipine is neuroprotective in a mouse model of Parkinson's disease. Neurobiol Dis, 2011. 43(2): p. 364-71.
23. Gombash, S. E., et al., Neuroprotective potential of pleiotrophin overexpression in the striatonigral pathway compared with overexpression in both the striatonigral and nigrostriatal pathways. Gene Ther, 2014. 21(7): p. 682-93.
24. Maries, E., et al., Focal not widespread grafts induce novel dyskinetic behavior in parkinsonian rats. Neurobiol Dis, 2006. 21(1): p. 165-80.
25. Schallert, T., Behavioral tests for preclinical intervention assessment. NeuroRx, 2006. 3(4): p. 497-504.
26. Grabinski, T. M., et al., A method for combining RNAscope in situ hybridization with immunohistochemistry in thick free-floating brain sections and primary neuronal cultures. PLoS One, 2015. 10(3): p. e0120120.
27. Lieu, C. A., et al., Dyskinesias do not develop after chronic intermittent levodopa therapy in clinically hemiparkinsonian rhesus monkeys. Parkinsonism Relat Disord, 2011. 17(1): p. 34-9.
28. Kordower, J. H., et al., Parkinsonian monkeys with prior levodopa-induced dyskinesias followed by fetal dopamine precursor grafts do not display graft-induced dyskinesias. J Comp Neurol, 2017. 525(3): p. 498-512.
29. Konradi, C., et al., Transcriptome analysis in a rat model of L-DOPA-induced dyskinesia. Neurobiol Dis, 2004. 17(2): p. 219-36.
30. Hassin-Baer, S., et al., Gender effect on time to levodopa-induced dyskinesias. J Neurol, 2011. 258(11): p. 2048-53.
31. Gombash, S. E., et al., Striatal pleiotrophin overexpression provides functional and morphological neuroprotection in the 6-hydroxydopamine model. Mol Ther, 2012. 20(3): p. 544-54.
32. Burger, C., et al., Systemic mannitol-induced hyperosmolality amplifies rAAV2-mediated striatal transduction to a greater extent than local co-infusion. Mol Ther, 2005. 11(2): p. 327-31.
33. Bhide, N., et al., Effects of the beta-adrenergic receptor antagonist Propranolol on dyskinesia and L-DOPA-induced striatal DA efflux in the hem i-parkinsonian rat. J Neurochem, 2015. 134(2): p. 222-32.
34. Lundblad, M., et al., Pharmacological validation of behavioural measures of akinesia and dyskinesia in a rat model of Parkinson's disease. Eur J Neurosci, 2002. 15(1): p. 120-32.
35. Levine, N. D., et al., Advances in thin tissue Golgi-Cox impregnation: fast, reliable methods for multi-assay analyses in rodent and non-human primate brain. J Neurosci Methods, 2013. 213(2): p. 214-27.
36. Soderstrom, K. E., et al., The synaptic impact of the host immune response in a parkinsonian allograft rat model: Influence on graft-derived aberrant behaviors. Neurobiol Dis, 2008. 32(2): p. 229-42.
37. Popov, V. I. and M. G. Stewart, Complexity of contacts between synaptic boutons and dendritic spines in adult rat hippocampus: three-dimensional reconstructions from serial ultrathin sections in vivo. Synapse, 2009. 63(5): p. 369-77.
38. Morin, N., et al., Contribution of brain serotonin subtype 1B receptors in levodopa-induced motor complications. Neuropharmacology, 2015. 99: p. 356-68.
39. Roussakis, A. A., et al., Serotonin-to-dopamine transporter ratios in Parkinson disease: Relevance for dyskinesias. Neurology, 2016. 86(12): p. 1152-8.
40. Rylander, D., The serotonin system: a potential target for anti-dyskinetic treatments and biomarker discovery. Parkinsonism Relat Disord, 2012. 18 Suppl 1: p. S126-8.
41. Rylander, D., et al., Maladaptive plasticity of serotonin axon terminals in levodopa-induced dyskinesia. Ann Neurol, 2010. 68(5): p. 619-28.
42. Belmer, A., et al., Mapping the connectivity of serotonin transporter immunoreactive axons to excitatory and inhibitory neurochemical synapses in the mouse limbic brain. Brain Struct Funct, 2017. 222(3): p. 1297-1314.
43. Andersson, M., A. Hilbertson, and M. A. Cenci, Striatal fosB expression is causally linked with I-DOPA-induced abnormal involuntary movements and the associated upregulation of striatal prodynorphin mRNA in a rat model of Parkinson's disease. Neurobiol Dis, 1999. 6(6): p. 461-74.
44. Cenci, M. A., Transcription factors involved in the pathogenesis of L-DOPA-induced dyskinesia in a rat model of Parkinson's disease. Amino Acids, 2002. 23(1-3): p. 105-9.
45. Lindgren, H. S., et al., Putaminal upregulation of FosB/DeltaFosB-like immunoreactivity in Parkinson's disease patients with dyskinesia. J Parkinsons Dis, 2011. 1(4): p. 347-57.
46. Heiman, M., et al., Molecular adaptations of striatal spiny projection neurons during levodopa-induced dyskinesia. Proc Natl Acad Sci USA, 2014. 111(12): p. 4578-83.
47. Sodersten, E., et al., Dopamine signaling leads to loss of Polycomb repression and aberrant gene activation in experimental parkinsonism. PLoS Genet, 2014. 10(9): p. e1004574.
48. Steece-Collier, K., et al., Embryonic mesencephalic grafts increase levodopa-induced forelimb hyperkinesia in parkinsonian rats. Mov Disord, 2003. 18(12): p. 1442-54.
49. Steece-Collier, K., et al., Effect of levodopa priming on dopamine neuron transplant efficacy and induction of abnormal involuntary movements in parkinsonian rats. J Comp Neurol, 2009. 515(1): p. 15-30.
50. Mercado N M, C. T., Sortwell C E and Steece-Collier K, BDNF in the Aged Brain: Translational Implications for Parkinson's Disease. Austin Neurol & Neurosci, 2017. 2(2): p. 1-8.
51. Veng, L. M., M. H. Mesches, and M. D. Browning, Age-related working memory impairment is correlated with increases in the L-type calcium channel protein alpha1D (Cav1.3) in area CA1 of the hippocampus and both are ameliorated by chronic nimodipine treatment. Brain Res Mol Brain Res, 2003. 110(2): p. 193-202.
52. Polinski, N. K., et al., Impact of age and vector construct on striatal and nigral transgene expression. Mol Ther Methods Clin Dev, 2016. 3: p. 16082.
53. Halje, P., et al., Levodopa-induced dyskinesia is strongly associated with resonant cortical oscillations. J Neurosci, 2012. 32(47): p. 16541-51.
54. Lindenbach, D., et al., The Role of Primary Motor Cortex (M1) Glutamate and GABA Signaling in I-DOPA-Induced Dyskinesia in Parkinsonian Rats. J Neurosci, 2016. 36(38): p. 9873-87.
55. Kordower, J. H., et al., Disease duration and the integrity of the nigrostriatal system in Parkinson's disease. Brain, 2013. 136(Pt 8): p. 2419-31.
56. Ko, W. K., Q. Li, and E. Bezard, Effects of L-tryptophan on L-DOPA-induced dyskinesia in the L-methyl-4-phenyl-1,2,3,6-tetrahydropyridine (MPTP)-treated macaque model of Parkinson's disease. Neurosci Lett, 2014. 566: p. 72-6.
57. Ko, W. K., et al., Combined fenobam and amantadine treatment promotes robust antidyskinetic effects in the 1-methyl-4-phenyl-1,2,3,6-tetrahydropyridine (MPTP)-lesioned primate model of Parkinson's disease. Mov Disord, 2014. 29(6): p. 772-9.
58. Kordower, J. H., et al., Neurodegeneration prevented by lentiviral vector delivery of GDNF in primate models of Parkinson's disease. Science, 2000. 290(5492): p. 767-73.
59. Kordower, J. H., et al., Delivery of neurturin by AAV2 (CERE-120)-mediated gene transfer provides structural and functional neuroprotection and neurorestoration in MPTP-treated monkeys. Ann Neurol, 2006. 60(6): p. 706-15.
60. Kordower, J. H., et al., Encapsulated PC12 cell transplants into hemiparkinsonian monkeys: a behavioral, neuroanatomical, and neurochemical analysis. Cell Transplant, 1995. 4(2): p. 155-71.
61. Bezard, E., et al., The mGluR5 negative allosteric modulator dipraglurant reduces dyskinesia in the MPTP macaque model. Mov Disord, 2014. 29(8): p. 1074-9.
62. Galvan, A. and Y. Smith, The primate thalamostriatal systems: Anatomical organization, functional roles and possible involvement in Parkinson's disease. Basal Ganglia, 2011. 1(4): p. 179-189.
63. Villalba, R. M., A. Mathai, and Y. Smith, Morphological changes of glutamatergic synapses in animal models of Parkinson's disease. Front Neuroanat, 2015. 9: p. 117.
64. Villalba, R. M. and Y. Smith, Loss and remodeling of striatal dendritic spines in Parkinson's disease: from homeostasis to maladaptive plasticity? J Neural Transm (Vienna), 2018. 125(3): p. 431-447.
65. Landis, S. C., et al., A call for transparent reporting to optimize the predictive value of preclinical research. Nature, 2012. 490(7419): p. 187-91.

SEQUENCES

| SEQ | | |
|---|---|---|
| ID. NO. | Sequence | Name |
| 1 | CGAGGCAAACTATGCAAGA | Cacna1d complement to |
| | | shRNA-Rat |
| 2 | ACTATGCAAGAGGCACCAGA | Cacna1d complement to |
| | | shRNA-Rat |
| 3 | GCGTCAGTGTGTGGAATAT | Cacna1d complement to |
| | | shRNA-Rat |
| 4 | GGCCATTGCTGTAGACAAT | Cacna1d complement to |
| | | shRNA-Rat |
| 5 | CGTGCCCTCTTCTGTTTAT | Cacna1d complement to |
| | | shRNA-Rat |
| 6 | CTCCTCGCCTTTCGAATAT | Cacna1d complement to |
| | | shRNA-Rat |
| 7 | CCCTGAAGATGATTCTAAT | Cacna1d complement to |
| | | shRNA-Rat |
| 8 | CCCGCTATTATGAAACTTA | Cacna1d complement to |
| | | shRNA-Rat |
| 9 | GAAGAGGCGCGGCCAAGAC | Partial rat shRNA |
| 10 | CAACAAGATGAAGAGCACC | Scrambled shRNA |
| 11 | AATTCATATTTGCATGTCGCTATGTGTTCTGGGAAATCACCATAAACGTGAAATG | Human H1 polymerase |
| | TCTTTGGATTTGGGAATCTTATAAGTTCTGTATGAGACACTCGGATCCG | promoter |
| 12 | ATGATGATGATGATGATGATGAAAAAAATGCAGCATCAACGGCAGCAGCAAGCGG | DNA sequence of |
| | ACCACGCGAACGAGGCAAACTATGCAAGAGGCACCAGACTCCCTCTTTCTGGTGA | cacna1d |
| | AGGACCAACTTCTCAGCCGAATAGCTCCAAGCAAACTGTCCTGTCTTGGCAAGCT | |
| | GCAATCGATGCTGCTAGACAGGCCAAGGCTGCCCAAACTATGAGCACCTCTGCAC | |
| | CCCGACCTGTAGGATCTCTCTCCCAAAGAAAACGTCAGCAATACGCCAAGAGCAA | |
| | AAAACAGGGTAACTCGTCCAACAGCCGACCTGCCCGCGCCCTTTTCTGTTTATCA | |
| | CTCAATAACCCCATCCGAAGAGCCTGCATTAGTATAGTGGAATGGAAACCATTTG | |
| | ACATATTTATATTATTGGCTATTTTTGCCAATTGTGTGGCCTTAGCTATTTACAT | |
| | CCCATTCCCTGAAGATGATTCTAATTCAACAAATCATAACTTGGAAAAAGTAGAA | |
| | TATGCCTTCCTGATTATTTTTACAGTCGAGACATTTTTGAAGATTATAGCGTATG | |
| | GATTATTGCTACATCCTAATGCTTATGTTAGGAATGGATGGAATTTACTGGATTT | |
| | TGTTATAGTAATAGTAGGATTGTTTAGTGTAATTTTGGAACAATTAACCAAAGAA | |
| | ACAGAAGGCGGGAACCACTCTAGCGGCAAGTCTGGAGGCTTTGATGTCAAAGCCC | |
| | TCCGTGCCTTTCGAGTGTTGCGACCACTTCGACTAGTGTCAGGAGTGCCCAGTTT | |
| | ACAAGTTGTCCTGAACTCCATTATAAAAGCCATGGTTCCCCTCCTTCACATAGCC | |
| | CTTTTGGTATTATTTGTAATCATAATCTATGCTATTATAGGATTGGAACTTTTTA | |
| | TTGGAAAAATGCACAAAACATGTTTTTTTGCTGACTCAGATATCGTAGCTGAAGA | |
| | GGACCCAGCTCCATGTGCGTTCTCAGGGAACGGACGCCAGTGTACCGCCAATGGC | |
| | ACGGAATGTAGGAGTGGCTGGGTCGGCCCGAACGGAGGCATCACCAACTTTGATA | |
| | ACTTTGCCTTTGCTATGCTCACTGTGTTTCAGTGCATCACCATGGAGGGCTGGAC | |
| | AGATGTGCTCTACTGGGTAAATGATGCGATAGGATGGGAATGGCCATGGGTGTAT | |
| | TTTGTTAGTCTCATCATCCTTGGCTCATTTTTCGTCCTTAACCTGGTTCTTGGTG | |
| | TCCTTAGTGGAGAATTCTCAAAGGAAAGAGAGAAGGCAAAAGCACGGGGAGATTT | |
| | CCAGAAGCTCCGGGAGAAGCAGCAGCTGGAGGAGGATCTAAAGGGCTACTTGGAT | |
| | TGGATCACCCAAGCTGAGGACATCGATCCCGAGAACGAGGAAGAAGGAGGAGAGG | |
| | AAGGCAAACGAAATACTAGCATGCCCACCAGTGAGACTGAGTCTGTGAACACAGA | |
| | GAACGTCAGCGGCGAAGGCGAGACCCGAGGCTGCTGTGGAAGTCTCTGGTGCTGG | |
| | TGGAGACGGAGAGGCGCGGCCAAGGCGGGGCCCTCTGGGTGTCGGCGGTGGGGGT | |
| | CAAGCCATCTCAAAATCCAAACTCAGCCGACGCTGGCGTCGCTGGAACCGATTCA | |
| | ATCGCAGAAGATGTAGGGCCGCCGTGAAG | |
| 13 | MMMMMMMKKMQHQRQQQADHANEANYARGTRLPLSGEGPTSQPNSSKQTVLSWQA | Protein sequence of |
| | AIDAARQAKAAQTMSTSAPPPVGSLSQRKRQQYAKSKKQGNSSNSRPARALFCLS | cacna1d |
| | LNNPIRRACISIVEWKPFDIFILLAIFANCVALAIYIPFPEDDSNSTNHNLEKVE | |
| | YAFLIIFTVETFLKIIAYGLLLHPNAYVRNGWNLLDFVIVIVGLFSVILEQLTKE | |
| | TEGGNHSSGKSGGFDVKALRAFRVLRPLRLVSGVPSLQVVLNSIIKAMVPLLHIA | |
| | LLVLFVIIIYAIIGLELFIGKMHKTCFFADSDIVAEEDPAPCAFSGNGRQCTANG | |
| | TECRSGWVGPNGGITNFDNFAFAMLTVFQCITMEGWTDVLYWMNDAMGFELPWVY | |
| | FVSLVIFGSFFVLNLVLGVLSGEFSKEREKAKARGDFQKLREKQQLEEDLKGYLD | |
| | WITQAEDIDPENEEEGGEEGKRNTSMPTSETESVNTENVSGEGENRGCCGSLCQA | |
| | ISKSKLSRRWRRWNRFNRRRCRAAVKSVTFYWLVIVLVFLNTLTISSEHYNQPDW | |
| | LTQIQDIANKVLLALFTCEMLVKMYSLGLQAYFVSLFNRFDCFVVCGGITETILV | |
| | ELEIMSPLGISVFRCVRLLRIFKVTRHWTSLSNLVASLLNSMKSIASLLLLLFLF | |
| | IIIFSLLGMQLFGGKFNFDETQTKRSTFDNFPQALLTVFQILTGEDWNAVMYDGI | |
| | MAYGGPSSSGMIVCIYFIILFICGNYILLNVFLAIAVDNLADAESLNTAQKEEAE | |
| | EKERKKIARKESLENKKNNKPEVNQIANSDNKVTIDDYREEDEDKDPYPPCDVPV | |
| | GEEEEEEEEDEPEVPAGPRPRRISELNMKEKIAPIPEGSAFFILSKTNPIRVGCH | |
| | KLINHHIFTNLILVFIMLSSAALAAEDPIRSHSFRNTILGYFDYAFTAIFTVEIL | |
| | LKMTTFGAFLHKGAFCRNYFNLLDMLVVGVSLVSFGIQSSAISVVKILRVLRVLR | |
| | PLRAINRAKGLKHVVQCVFVAIRTIGNIMIVTTLLQFMFACIGVQLFKGKFYRCT | |
| | DEAKSNPEECRGLFILYKDGDVDSPVVRERIWQNSDFNFDNVLSAMMALFTVSTF | |
| | EGWPALLYKAIDSNGENIGPIYNHRVEISIFFIIYIIIVAFFMMNIFVGFVIVTF | |
| | QEQGEKEYKNCELDKNQRQCVEYALKARPLRRYIPKNPYQYKFWYVVNSSPFEYM | |
| | MFVLIMLNTLCLAMQHYEQSKMFNDAMDILNMVFTGVFTVEMVLKVIAFKPKGYF | |
| | SDAWNTFDSLIVIGSIIDVALSEADPTESENVPVPTATPGENSEESNRISITFFR | |
| | LFRVMRLVKLLSRGEGIRTLLWTFIKSFQALPYVALLIAMLFFIYAVIGMQMFGK | |
| | VAMRDNNQINRNNNFQTFPQAVLLLFRCATGEAWQEIMLACLPGKLCDPESDYNP | |
| | GEEYTCGSNFAIVYFISFYMLCAFLIINLFVAVIMDNFDYLTRDWSILGPHHLDE | |
| | FKRIWSEYDPEAKGRIKHLDVVTLLRRIQPPLGFGKLCPHRVACKRLVAMNMPLN | |
| | SDGTVMFNATLFALVRTALKIKTEGNLEQANEELRAVIKKIWKKTSMKLLDQVVP | |
| | PAGDDEVTVGKFYATFLIQDYFRKFKKRKEQGLVGKYPAKNTTIALQAGLRTLHD | |
| | IGPEIRRAISCDLQDDEPEETKREEEDDVFKRNGALLGNHVNHVNSDRRDSLQQT | |
| | NTTHRPLHVQRPSIPPASDTEKPLFPPAGNSVCHNHHNHNSIGKQVPTSTNANLN | |
| | NANMSKAAHGKRPSIGNLEHVSENGHHSSHKHDREPQRRSSVKRTRYYETYIRSD | |
| | SGDEQLPTICREDPEIHGYFRDPHCLGEQEYFSSEECYEDDSSPTWSRQNYGYYS | |
| | RYPGRNIDSERPRGYHHPQGFLEDDDSPVCYDSRRSPRRRLLPPTPASHRRSSFN | |
| | FECLRRQSSQEEVPSSPIFPHRTALPLHLMQQQIMAVAGLDSSKAQKYSPSHSTR | |
| | SWATPPATPPYRDWTPCYTPLIQVEQSEALDQVNGSLPSLHRSSWYTDEPDISYR | |
| | TFTPASLTVPSSFRNKNSDKQRSADSLVEAVLISEGLGRYARDPKFVSATKHEIA | |
| | DACDLTIDEMESAASTLLNGNVRPRANGDVGPLSHRQDYELQDFGPGYSDEEPDP | |
| | GRDEEDLADEMICITTL | |
| 14 | GAAGAGGCGCGGCCAAGACTTCAAGAGAGTCTTGGCCGCGCCTCTTC | Rat shRNA |
| 15 | GCGAGGCAAACTATGCAAGATTCAAGAGATCTTGCATAGTTTGCCTCG | Human shRNA-1 |
| 16 | GAGGAAGGCAAACGAAATATTCAAGAGATATTTCGTTTGCCTTCCTC | Human shRNA-2 |
| 17 | GCCCTGAAGATGATTCTAATTTCAAGAGAATTAGAATCATCTTCAGGG | Human shRNA-3 |
| 18 | GTCCTGAACTCCATTATAATTCAAGAGATTATAATGGAGTTCAGGAC | Human shRNA-4 |
| 19 | GAGGATCTAAAGGGCTACTTTCAAGAGAAGTAGCCCTTTAGATCCTC | Human shRNA-5 |
| 20 | ttttttggaaa | terminator |
| 21 | aattcatatttgcatgtcgctatgtgttctgggaaatcaccataaacgtgaaatg | Rat shRNA expression |
| | tctttggatttgggaatcttataagttctgtatgagaccactcggatccgaagag | construct |
| | gcgcggccaagacttcaagagagtcttggccgcgcctcttcttttttggaaa | |
| 22 | aattcatatttgcatgtcgctatgtgttctgggaaatcaccataaacgtgaaatg | Human shRNA |
| | tctttggatttgggaatcttataagttctgtatgagaccactcggatccGCGAGG | expression construct |
| | CAAACTATGCAAGATTCAAGAGATCTTGCATAGTTTGCCTCGttttttggaaa | |
| 23 | aattcatatttgcatgtcgctatgtgttctgggaaatcaccataaacgtgaaatg | Human shRNA |
| | tctttggatttgggaatcttataagttctgtatgagaccactcggatccGAGGAA | expression construct |
| | GGCAAACGAAATATTCAAGAGATATTTCGTTTGCCTTCCTCttttttggaaa | |
| 24 | aattcatatttgcatgtcgctatgtgttctgggaaatcaccataaacgtgaaatg | Human shRNA |
| | tctttggatttgggaatcttataagttctgtatgagaccactcggatccGTCCTG | expression construct |
| | AACTCCATTATAATTCAAGAGATTATAATGGAGTTCAGGACttttttggaaa | |
| 25 | aattcatatttgcatgtcgctatgtgttctgggaaatcaccataaacgtgaaatg | Human shRNA |
| | tctttggatttgggaatcttataagttctgtatgagaccactcggatccGAGGAT | expression construct |
| | CTAAAGGGCTACTTTCAAGAGAAGTAGCCCTTTAGATCCTCttttttggaaa | |
| 26 | aattcatatttgcatgtcgctatgtgttctgggaaatcaccataaacgtgaaatg | Human shRNA |
| | tctttggatttgggaatcttataagttctgtatgagaccactcggatccGCCCTG | expression construct |
| | AAGATGATTCTAATTTCAAGAGAATTAGAATCATCTTCAGGG ttttttggaaa | |
| 27 | ttggccactccctctctgcgcgctcgctcgctcactgaggccgggcgaccaaagg | AAV9.H1.shRNA- |
| | tcgcccgacgcccgggctttgcccgggcggcctcagtgagcgagcgagcgcgcag | CaV1.3-Rat |
| | agagggagtggccaactccatcactaggggttcctagatctgaattcggtgctag | |
| | caccctagttattaatagtaatcaattacggggtcattagttcatagcccatata | |
| | tggagttccgcgttacataacttacggtaaatggcccgcctggctgaccgcccaa | |
| | cgacccccgcccattgacgtcaataatgacgtatgttcccatagtaacgccaata | |
| | gggactttccattgacgtcaatgggtggactatttacggtaaactgcccacttgg | |
| | cagtacatcaagtgtatcatatgccaagtacgccccctattgacgtcaatgacgg | |
| | taaatggcccgcctggcattatgcccagtacatgaccttatgggactttcctact | |
| | tggcagtacatctacgtattagtcatcgctattaccatggtcgaggtgagcccca | |
| | cgttctgcttcactctccccatctcccccccctccccacccccaattttgtattt | |
| | atttattttttaattattttgtgcagcgatgggggcggggggggggggggggcgc | |
| | gcgccaggcggggcggggcggggcgaggggcggggcggggcgaggcggagaggtg | |
| | cggcggcagccaatcagagcggcgcgctccgaaagtttccttttatggcgaggcg | |
| | gcggcggcggcggccctataaaaagcgaagcgcgcggcgggcgggagtcgctgcg | |
| | acgctgccttcgccccgtgccccgctccgccgccgcctcgcgccgcccgccccgg | |
| | ctctgactgaccgcgttactcccacaggtgagcgggcgggacggcccttctcctc | |
| | cgggctgtaattagcgcttggtttaatgacggcttgtttcttttctgtggctgcg | |
| | tgaaagccttgaggggctccgggagggccctttgtgcgggggggagcggctcggg | |
| | gggtgcgtgcgtgtgtgtgtgcgtggggagcgccgcgtgcggcccgcgctgcccg | |
| | gcggctgtgagcgctgcgggcgcggcgcggggctttgtgcgctccgcagtgtgcg | |
| | cgaggggagcgcggccgggggcggtgccccgcggtgcggggggggctgcgagggg | |
| | aacaaaggctgcgtgcggggtgtgtgcgtgggggggtgagcagggggtgtgggcg | |
| | cggcggtcgggctgtaacccccccctgcacccccctccccgagttgctgagcacg | |
| | gcccggcttcgggtgcggggctccgtacggggcgtggcgcggggctcgccgtgcc | |
| | gggcggggggtggcggcaggtgggggtgccgggcggggcggggccgcctcgggcc | |
| | ggggagggctcgggggaggggcgcggcggcccccggagcgccggcggctgtcgag | |
| | gcgcggcgagccgcagccattgccttttatggtaatcgtgcgagagggcgcaggg | |
| | acttcctttgtcccaaatctgtgcggagccgaaatctgggaggcgccgccgcacc | |
| | ccctctagcgggcgcggggcgaagcggtgcggcgccggcaggaaggaaatgggcg | |
| | gggagggccttcgtgcgtcgccgcgccgccgtccccttctccctctccagcctcg | |
| | gggctgtccgcggggggacggctgccttcgggggggacggggcagggcggggttc | |
| | ggcttctggcgtgtgaccggcggctctagagcctctgctaaccatgttcatgcct | |
| | tcttctttttcctacagctcctgggcaacgtgctggttattgtgctgtctcatca | |
| | ttttggcaaagtattcctcgaagatctgctagcaggcgcgcggccgccgccacca | |
| | tgagcaagggcgaggaactgttcactggcgtggtcccaattctcgtggaactgga | |
| | tggcgatgtgaatgggcacaaattttctgtcagcggagagggtgaaggtgatgcc | |
| | acatacggaaagctcaccctgaaattcatctgcaccactggaaagctccctgtgc | |
| | catggccaacactggtcactaccctgacctatggcgtgcagtgcttttccagata | |
| | cccagaccatatgaagcagcatgactttttcaagagcgccatgcccgagggctat | |
| | gtgcaggagagaaccatctttttcaaagatgacgggaactacaagacccgcgctg | |
| | aagtcaagttcgaaggtgacaccctggtgaatagaatcgagctgaagggcattga | |
| | ctttaaggaggatggaaacattctcggccacaagctggaatacaactataactcc | |
| | cacaatgtgtacatcatggccgacaagcaaaagaatggcatcaaggtcaacttca | |
| | agatcagacacaacattgaggatggatccgtgcagctggccgaccattatcaaca | |
| | gaacactccaatcggcgacggccctgtgctcctcccagacaaccattacctgtcc | |
| | acccagtctgccctgtctaaagatcccaacgaaaagagagaccacatggtcctgc | |
| | tggagtttgtgaccgctgctgggatcacacatggcatggacgagctgtacaagtg | |
| | agcggccgcggggttccagacatgataagatacattgatgagtttggacaaacca | |
| | caactagaatgcagtgaaaaaaatgctttatttgtgaaatttgtgatgctattgc | |
| | tttatttgtaaccattataagctgcaataaacaagttaacaacaacaattgcatt | |
| | cattttatgtttcaggttcagggggaggtgtgggaggttttttactagtaattca | |
| | tatttgcatgtcgctatgtgttctgggaaatcaccataaacgtgaaatgtctttg | |
| | gatttgggaatcttataagttctgtatgagaccactcggatccgaagaggcgcgg | |
| | ccaagacttcaagagagtcttggccgcgcctcttcttttttggaaaagcttgtcg | |
| | actagagctcgctgatcagcctcgactgtgccttctagttgccagccatctgttg | |
| | tttgcccctcccccgtgccttccttgaccctggaaggtgccactcccactgtcct | |
| | ttcctaataaaatgaggaaattgcatcgcattgtctgagtaggtgtcattctatt | |
| | ctggggggtggggtggggcaggacagcaagggggaggattgggaagacaatagca | |
| | ggcatgctggggagagatctaggaacccctagtgatggagttggccactccctct | |
| | ctgcgcgctcgctcgctcactgaggccgcccgggcaaagcccgggcgtcgggcga | |
| | cctttggtcgcccggcctcagtgagcgagcgagcgcgcagagagggagtggccaa | |
| | c | |
| 28 | ttggccactccctctctgcgcgctcgctcgctcactgaggccgggcgaccaaagg | AAV9.H1.shRNA- |
| | tcgcccgacgcccgggctttgcccgggcggcctcagtgagcgagcgagcgcgcag | CaV1.3-Human-1 |
| | agagggagtggccaactccatcactaggggttcctagatctgaattcggtgctag | |
| | caccctagttattaatagtaatcaattacggggtcattagttcatagcccatata | |
| | tggagttccgcgttacataacttacggtaaatggcccgcctggctgaccgcccaa | |
| | cgacccccgcccattgacgtcaataatgacgtatgttcccatagtaacgccaata | |
| | gggactttccattgacgtcaatgggtggactatttacggtaaactgcccacttgg | |
| | cagtacatcaagtgtatcatatgccaagtacgccccctattgacgtcaatgacgg | |
| | taaatggcccgcctggcattatgcccagtacatgaccttatgggactttcctact | |
| | tggcagtacatctacgtattagtcatcgctattaccatggtcgaggtgagcccca | |
| | cgttctgcttcactctccccatctcccccccctccccacccccaattttgtattt | |
| | atttattttttaattattttgtgcagcgatgggggcggggggggggggggggcgc | |
| | gcgccaggcggggcggggcggggcgaggggcggggcggggcgaggcggagaggtg | |
| | cggcggcagccaatcagagcggcgcgctccgaaagtttccttttatggcgaggcg | |
| | gcggcggcggcggccctataaaaagcgaagcgcgcggcgggcgggagtcgctgcg | |
| | acgctgccttcgccccgtgccccgctccgccgccgcctcgcgccgcccgccccgg | |
| | ctctgactgaccgcgttactcccacaggtgagcgggcgggacggcccttctcctc | |
| | cgggctgtaattagcgcttggtttaatgacggcttgtttcttttctgtggctgcg | |
| | tgaaagccttgaggggctccgggagggccctttgtgcgggggggagcggctcggg | |
| | gggtgcgtgcgtgtgtgtgtgcgtggggagcgccgcgtgcggcccgcgctgcccg | |
| | gcggctgtgagcgctgcgggcgcggcgcggggctttgtgcgctccgcagtgtgcg | |
| | cgaggggagcgcggccgggggcggtgccccgcggtgcggggggggctgcgagggg | |
| | aacaaaggctgcgtgcggggtgtgtgcgtgggggggtgagcagggggtgtgggcg | |
| | cggcggtcgggctgtaacccccccctgcacccccctccccgagttgctgagcacg | |
| | gcccggcttcgggtgcggggctccgtacggggcgtggcgcggggctcgccgtgcc | |
| | gggcggggggtggcggcaggtgggggtgccgggcggggcggggccgcctcgggcc | |
| | ggggagggctcgggggaggggcgcggcggcccccggagcgccggcggctgtcgag | |
| | gcgcggcgagccgcagccattgccttttatggtaatcgtgcgagagggcgcaggg | |
| | acttcctttgtcccaaatctgtgcggagccgaaatctgggaggcgccgccgcacc | |
| | ccctctagcgggcgcggggcgaagcggtgcggcgccggcaggaaggaaatgggcg | |
| | gggagggccttcgtgcgtcgccgcgccgccgtccccttctccctctccagcctcg | |
| | gggctgtccgcggggggacggctgccttcgggggggacggggcagggcggggttc | |
| | ggcttctggcgtgtgaccggcggctctagagcctctgctaaccatgttcatgcct | |
| | tcttctttttcctacagctcctgggcaacgtgctggttattgtgctgtctcatca | |
| | ttttggcaaagtattcctcgaagatctgctagcaggcgcgcggccgccgccacca | |
| | tgagcaagggcgaggaactgttcactggcgtggtcccaattctcgtggaactgga | |
| | tggcgatgtgaatgggcacaaattttctgtcagcggagagggtgaaggtgatgcc | |
| | acatacggaaagctcaccctgaaattcatctgcaccactggaaagctccctgtgc | |
| | catggccaacactggtcactaccctgacctatggcgtgcagtgcttttccagata | |
| | cccagaccatatgaagcagcatgactttttcaagagcgccatgcccgagggctat | |
| | gtgcaggagagaaccatctttttcaaagatgacgggaactacaagacccgcgctg | |
| | aagtcaagttcgaaggtgacaccctggtgaatagaatcgagctgaagggcattga | |
| | ctttaaggaggatggaaacattctcggccacaagctggaatacaactataactcc | |
| | cacaatgtgtacatcatggccgacaagcaaaagaatggcatcaaggtcaacttca | |
| | agatcagacacaacattgaggatggatccgtgcagctggccgaccattatcaaca | |
| | gaacactccaatcggcgacggccctgtgctcctcccagacaaccattacctgtcc | |
| | acccagtctgccctgtctaaagatcccaacgaaaagagagaccacatggtcctgc | |
| | tggagtttgtgaccgctgctgggatcacacatggcatggacgagctgtacaagtg | |
| | agcggccgcggggttccagacatgataagatacattgatgagtttggacaaacca | |
| | caactagaatgcagtgaaaaaaatgctttatttgtgaaatttgtgatgctattgc | |
| | tttatttgtaaccattataagctgcaataaacaagttaacaacaacaattgcatt | |
| | cattttatgtttcaggttcagggggaggtgtgggaggttttttactagtaattca | |
| | tatttgcatgtcgctatgtgttctgggaaatcaccataaacgtgaaatgtctttg | |
| | gatttgggaatcttataagttctgtatgagaccactcggatccGCGAGGCAAACT | |
| | ATGCAAGATTCAAGAGATCTTGCATAGTTTGCCTCGttttttggaaaagcttgtc | |
| | gactagagctcgctgatcagcctcgactgtgccttctagttgccagccatctgtt | |
| | gtttgcccctcccccgtgccttccttgaccctggaaggtgccactcccactgtcc | |
| | tttcctaataaaatgaggaaattgcatcgcattgtctgagtaggtgtcattctat | |
| | tctggggggtggggtggggcaggacagcaagggggaggattgggaagacaatagc | |
| | aggcatgctggggagagatctaggaacccctagtgatggagttggccactccctc | |
| | tctgcgcgctcgctcgctcactgaggccgcccgggcaaagcccgggcgtcgggcg | |
| | acctttggtcgcccggcctcagtgagcgagcgagcgcgcagagagggagtggcca | |
| | ac | |
| 29 | ttggccactccctctctgcgcgctcgctcgctcactgaggccgggcgaccaaagg | AAV9.H1.shRNA- |
| | tcgcccgacgcccgggctttgcccgggcggcctcagtgagcgagcgagcgcgcag | CaV1.3-Human-2 |
| | agagggagtggccaactccatcactaggggttcctagatctgaattcggtgctag | |
| | caccctagttattaatagtaatcaattacggggtcattagttcatagcccatata | |
| | tggagttccgcgttacataacttacggtaaatggcccgcctggctgaccgcccaa | |
| | cgacccccgcccattgacgtcaataatgacgtatgttcccatagtaacgccaata | |
| | gggactttccattgacgtcaatgggtggactatttacggtaaactgcccacttgg | |
| | cagtacatcaagtgtatcatatgccaagtacgccccctattgacgtcaatgacgg | |
| | taaatggcccgcctggcattatgcccagtacatgaccttatgggactttcctact | |
| | tggcagtacatctacgtattagtcatcgctattaccatggtcgaggtgagcccca | |
| | cgttctgcttcactctccccatctcccccccctccccacccccaattttgtattt | |
| | atttattttttaattattttgtgcagcgatgggggcggggggggggggggggcgc | |
| | gcgccaggcggggcggggcggggcgaggggcggggcggggcgaggcggagaggtg | |
| | cggcggcagccaatcagagcggcgcgctccgaaagtttccttttatggcgaggcg | |
| | gcggcggcggcggccctataaaaagcgaagcgcgcggcgggcgggagtcgctgcg | |
| | acgctgccttcgccccgtgccccgctccgccgccgcctcgcgccgcccgccccgg | |
| | ctctgactgaccgcgttactcccacaggtgagcgggcgggacggcccttctcctc | |
| | cgggctgtaattagcgcttggtttaatgacggcttgtttcttttctgtggctgcg | |
| | tgaaagccttgaggggctccgggagggccctttgtgcgggggggagcggctcggg | |
| | gggtgcgtgcgtgtgtgtgtgcgtggggagcgccgcgtgcggcccgcgctgcccg | |
| | gcggctgtgagcgctgcgggcgcggcgcggggctttgtgcgctccgcagtgtgcg | |
| | cgaggggagcgcggccgggggcggtgccccgcggtgcggggggggctgcgagggg | |
| | aacaaaggctgcgtgcggggtgtgtgcgtgggggggtgagcagggggtgtgggcg | |
| | cggcggtcgggctgtaacccccccctgcacccccctccccgagttgctgagcacg | |
| | gcccggcttcgggtgcggggctccgtacggggcgtggcgcggggctcgccgtgcc | |
| | gggcggggggtggcggcaggtgggggtgccgggcggggcggggccgcctcgggcc | |
| | ggggagggctcgggggaggggcgcggcggcccccggagcgccggcggctgtcgag | |
| | gcgcggcgagccgcagccattgccttttatggtaatcgtgcgagagggcgcaggg | |
| | acttcctttgtcccaaatctgtgcggagccgaaatctgggaggcgccgccgcacc | |
| | ccctctagcgggcgcggggcgaagcggtgcggcgccggcaggaaggaaatgggcg | |
| | gggagggccttcgtgcgtcgccgcgccgccgtccccttctccctctccagcctcg | |
| | gggctgtccgcggggggacggctgccttcgggggggacggggcagggcggggttc | |
| | ggcttctggcgtgtgaccggcggctctagagcctctgctaaccatgttcatgcct | |
| | tcttctttttcctacagctcctgggcaacgtgctggttattgtgctgtctcatca | |
| | ttttggcaaagtattcctcgaagatctgctagcaggcgcgcggccgccgccacca | |
| | tgagcaagggcgaggaactgttcactggcgtggtcccaattctcgtggaactgga | |
| | tggcgatgtgaatgggcacaaattttctgtcagcggagagggtgaaggtgatgcc | |
| | acatacggaaagctcaccctgaaattcatctgcaccactggaaagctccctgtgc | |
| | catggccaacactggtcactaccctgacctatggcgtgcagtgcttttccagata | |
| | cccagaccatatgaagcagcatgactttttcaagagcgccatgcccgagggctat | |
| | gtgcaggagagaaccatctttttcaaagatgacgggaactacaagacccgcgctg | |
| | aagtcaagttcgaaggtgacaccctggtgaatagaatcgagctgaagggcattga | |
| | ctttaaggaggatggaaacattctcggccacaagctggaatacaactataactcc | |
| | cacaatgtgtacatcatggccgacaagcaaaagaatggcatcaaggtcaacttca | |
| | agatcagacacaacattgaggatggatccgtgcagctggccgaccattatcaaca | |
| | gaacactccaatcggcgacggccctgtgctcctcccagacaaccattacctgtcc | |
| | acccagtctgccctgtctaaagatcccaacgaaaagagagaccacatggtcctgc | |
| | tggagtttgtgaccgctgctgggatcacacatggcatggacgagctgtacaagtg | |
| | agcggccgcggggttccagacatgataagatacattgatgagtttggacaaacca | |
| | caactagaatgcagtgaaaaaaatgctttatttgtgaaatttgtgatgctattgc | |
| | tttatttgtaaccattataagctgcaataaacaagttaacaacaacaattgcatt | |
| | cattttatgtttcaggttcagggggaggtgtgggaggttttttactagtaattca | |
| | tatttgcatgtcgctatgtgttctgggaaatcaccataaacgtgaaatgtctttg | |
| | gatttgggaatcttataagttctgtatgagaccactcggatccGAGGAAGGCAAA | |
| | CGAAATATTCAAGAGATATTTCGTTTGCCTTCCTCttttttggaaaagcttgtcg | |
| | actagagctcgctgatcagcctcgactgtgccttctagttgccagccatctgttg | |
| | tttgcccctcccccgtgccttccttgaccctggaaggtgccactcccactgtcct | |
| | ttcctaataaaatgaggaaattgcatcgcattgtctgagtaggtgtcattctatt | |
| | ctggggggtggggtggggcaggacagcaagggggaggattgggaagacaatagca | |
| | ggcatgctggggagagatctaggaacccctagtgatggagttggccactccctct | |
| | ctgcgcgctcgctcgctcactgaggccgcccgggcaaagcccgggcgtcgggcga | |
| | cctttggtcgcccggcctcagtgagcgagcgagcgcgcagagagggagtggccaa | |
| | c | |
| 30 | ttggccactccctctctgcgcgctcgctcgctcactgaggccgggcgaccaaagg | AAV9.H1.shRNA- |
| | tcgcccgacgcccgggctttgcccgggcggcctcagtgagcgagcgagcgcgcag | CaV1.3-Human-3 |
| | agagggagtggccaactccatcactaggggttcctagatctgaattcggtgctag | |
| | caccctagttattaatagtaatcaattacggggtcattagttcatagcccatata | |
| | tggagttccgcgttacataacttacggtaaatggcccgcctggctgaccgcccaa | |
| | cgacccccgcccattgacgtcaataatgacgtatgttcccatagtaacgccaata | |
| | gggactttccattgacgtcaatgggtggactatttacggtaaactgcccacttgg | |
| | cagtacatcaagtgtatcatatgccaagtacgccccctattgacgtcaatgacgg | |
| | taaatggcccgcctggcattatgcccagtacatgaccttatgggactttcctact | |
| | tggcagtacatctacgtattagtcatcgctattaccatggtcgaggtgagcccca | |
| | cgttctgcttcactctccccatctcccccccctccccacccccaattttgtattt | |
| | atttattttttaattattttgtgcagcgatgggggcggggggggggggggggcgc | |
| | gcgccaggcggggcggggcggggcgaggggcggggcggggcgaggcggagaggtg | |
| | cggcggcagccaatcagagcggcgcgctccgaaagtttccttttatggcgaggcg | |
| | gcggcggcggcggccctataaaaagcgaagcgcgcggcgggcgggagtcgctgcg | |
| | acgctgccttcgccccgtgccccgctccgccgccgcctcgcgccgcccgccccgg | |
| | ctctgactgaccgcgttactcccacaggtgagcgggcgggacggcccttctcctc | |
| | cgggctgtaattagcgcttggtttaatgacggcttgtttcttttctgtggctgcg | |
| | tgaaagccttgaggggctccgggagggccctttgtgcgggggggagcggctcggg | |
| | gggtgcgtgcgtgtgtgtgtgcgtggggagcgccgcgtgcggcccgcgctgcccg | |
| | gcggctgtgagcgctgcgggcgcggcgcggggctttgtgcgctccgcagtgtgcg | |
| | cgaggggagcgcggccgggggcggtgccccgcggtgcggggggggctgcgagggg | |
| | aacaaaggctgcgtgcggggtgtgtgcgtgggggggtgagcagggggtgtgggcg | |
| | cggcggtcgggctgtaacccccccctgcacccccctccccgagttgctgagcacg | |
| | gcccggcttcgggtgcggggctccgtacggggcgtggcgcggggctcgccgtgcc | |
| | gggcggggggtggcggcaggtgggggtgccgggcggggcggggccgcctcgggcc | |
| | ggggagggctcgggggaggggcgcggcggcccccggagcgccggcggctgtcgag | |
| | gcgcggcgagccgcagccattgccttttatggtaatcgtgcgagagggcgcaggg | |
| | acttcctttgtcccaaatctgtgcggagccgaaatctgggaggcgccgccgcacc | |
| | ccctctagcgggcgcggggcgaagcggtgcggcgccggcaggaaggaaatgggcg | |
| | gggagggccttcgtgcgtcgccgcgccgccgtccccttctccctctccagcctcg | |
| | gggctgtccgcggggggacggctgccttcgggggggacggggcagggcggggttc | |
| | ggcttctggcgtgtgaccggcggctctagagcctctgctaaccatgttcatgcct | |
| | tcttctttttcctacagctcctgggcaacgtgctggttattgtgctgtctcatca | |
| | ttttggcaaagtattcctcgaagatctgctagcaggcgcgcggccgccgccacca | |
| | tgagcaagggcgaggaactgttcactggcgtggtcccaattctcgtggaactgga | |
| | tggcgatgtgaatgggcacaaattttctgtcagcggagagggtgaaggtgatgcc | |
| | acatacggaaagctcaccctgaaattcatctgcaccactggaaagctccctgtgc | |
| | catggccaacactggtcactaccctgacctatggcgtgcagtgcttttccagata | |
| | cccagaccatatgaagcagcatgactttttcaagagcgccatgcccgagggctat | |
| | gtgcaggagagaaccatctttttcaaagatgacgggaactacaagacccgcgctg | |
| | aagtcaagttcgaaggtgacaccctggtgaatagaatcgagctgaagggcattga | |
| | ctttaaggaggatggaaacattctcggccacaagctggaatacaactataactcc | |
| | cacaatgtgtacatcatggccgacaagcaaaagaatggcatcaaggtcaacttca | |
| | agatcagacacaacattgaggatggatccgtgcagctggccgaccattatcaaca | |
| | gaacactccaatcggcgacggccctgtgctcctcccagacaaccattacctgtcc | |
| | acccagtctgccctgtctaaagatcccaacgaaaagagagaccacatggtcctgc | |
| | tggagtttgtgaccgctgctgggatcacacatggcatggacgagctgtacaagtg | |
| | agcggccgcggggttccagacatgataagatacattgatgagtttggacaaacca | |
| | caactagaatgcagtgaaaaaaatgctttatttgtgaaatttgtgatgctattgc | |
| | tttatttgtaaccattataagctgcaataaacaagttaacaacaacaattgcatt | |
| | cattttatgtttcaggttcagggggaggtgtgggaggttttttactagtaattca | |
| | tatttgcatgtcgctatgtgttctgggaaatcaccataaacgtgaaatgtctttg | |
| | gatttgggaatcttataagttctgtatgagaccactcggatccGCCCTGAAGATG | |
| | ATTCTAATTTCAAGAGAATTAGAATCATCTTCAGGGttttttggaaaagcttgtc | |
| | gactagagctcgctgatcagcctcgactgtgccttctagttgccagccatctgtt | |
| | gtttgcccctcccccgtgccttccttgaccctggaaggtgccactcccactgtcc | |
| | tttcctaataaaatgaggaaattgcatcgcattgtctgagtaggtgtcattctat | |
| | tctggggggtggggtggggcaggacagcaagggggaggattgggaagacaatagc | |
| | aggcatgctggggagagatctaggaacccctagtgatggagttggccactccctc | |
| | tctgcgcgctcgctcgctcactgaggccgcccgggcaaagcccgggcgtcgggcg | |
| | acctttggtcgcccggcctcagtgagcgagcgagcgcgcagagagggagtggcca | |
| | ac | |
| 31 | ttggccactccctctctgcgcgctcgctcgctcactgaggccgggcgaccaaagg | AAV9.H1.shRNA- |
| | tcgcccgacgcccgggctttgcccgggcggcctcagtgagcgagcgagcgcgcag | CaV1.3-Human-4 |
| | agagggagtggccaactccatcactaggggttcctagatctgaattcggtgctag | |
| | caccctagttattaatagtaatcaattacggggtcattagttcatagcccatata | |
| | tggagttccgcgttacataacttacggtaaatggcccgcctggctgaccgcccaa | |
| | cgacccccgcccattgacgtcaataatgacgtatgttcccatagtaacgccaata | |
| | gggactttccattgacgtcaatgggtggactatttacggtaaactgcccacttgg | |
| | cagtacatcaagtgtatcatatgccaagtacgccccctattgacgtcaatgacgg | |
| | taaatggcccgcctggcattatgcccagtacatgaccttatgggactttcctact | |
| | tggcagtacatctacgtattagtcatcgctattaccatggtcgaggtgagcccca | |
| | cgttctgcttcactctccccatctcccccccctccccacccccaattttgtattt | |
| | atttattttttaattattttgtgcagcgatgggggcggggggggggggggggcgc | |
| | gcgccaggcggggcggggcggggcgaggggcggggcggggcgaggcggagaggtg | |
| | cggcggcagccaatcagagcggcgcgctccgaaagtttccttttatggcgaggcg | |
| | gcggcggcggcggccctataaaaagcgaagcgcgcggcgggcgggagtcgctgcg | |
| | acgctgccttcgccccgtgccccgctccgccgccgcctcgcgccgcccgccccgg | |
| | ctctgactgaccgcgttactcccacaggtgagcgggcgggacggcccttctcctc | |
| | cgggctgtaattagcgcttggtttaatgacggcttgtttcttttctgtggctgcg | |
| | tgaaagccttgaggggctccgggagggccctttgtgcgggggggagcggctcggg | |
| | gggtgcgtgcgtgtgtgtgtgcgtggggagcgccgcgtgcggcccgcgctgcccg | |
| | gcggctgtgagcgctgcgggcgcggcgcggggctttgtgcgctccgcagtgtgcg | |
| | cgaggggagcgcggccgggggcggtgccccgcggtgcggggggggctgcgagggg | |
| | aacaaaggctgcgtgcggggtgtgtgcgtgggggggtgagcagggggtgtgggcg | |
| | cggcggtcgggctgtaacccccccctgcacccccctccccgagttgctgagcacg | |
| | gcccggcttcgggtgcggggctccgtacggggcgtggcgcggggctcgccgtgcc | |
| | gggcggggggtggcggcaggtgggggtgccgggcggggcggggccgcctcgggcc | |
| | ggggagggctcgggggaggggcgcggcggcccccggagcgccggcggctgtcgag | |
| | gcgcggcgagccgcagccattgccttttatggtaatcgtgcgagagggcgcaggg | |
| | acttcctttgtcccaaatctgtgcggagccgaaatctgggaggcgccgccgcacc | |
| | ccctctagcgggcgcggggcgaagcggtgcggcgccggcaggaaggaaatgggcg | |
| | gggagggccttcgtgcgtcgccgcgccgccgtccccttctccctctccagcctcg | |
| | gggctgtccgcggggggacggctgccttcgggggggacggggcagggcggggttc | |
| | ggcttctggcgtgtgaccggcggctctagagcctctgctaaccatgttcatgcct | |
| | tcttctttttcctacagctcctgggcaacgtgctggttattgtgctgtctcatca | |
| | ttttggcaaagtattcctcgaagatctgctagcaggcgcgcggccgccgccacca | |
| | tgagcaagggcgaggaactgttcactggcgtggtcccaattctcgtggaactgga | |
| | tggcgatgtgaatgggcacaaattttctgtcagcggagagggtgaaggtgatgcc | |
| | acatacggaaagctcaccctgaaattcatctgcaccactggaaagctccctgtgc | |
| | catggccaacactggtcactaccctgacctatggcgtgcagtgcttttccagata | |
| | cccagaccatatgaagcagcatgactttttcaagagcgccatgcccgagggctat | |
| | gtgcaggagagaaccatctttttcaaagatgacgggaactacaagacccgcgctg | |
| | aagtcaagttcgaaggtgacaccctggtgaatagaatcgagctgaagggcattga | |
| | ctttaaggaggatggaaacattctcggccacaagctggaatacaactataactcc | |
| | cacaatgtgtacatcatggccgacaagcaaaagaatggcatcaaggtcaacttca | |
| | agatcagacacaacattgaggatggatccgtgcagctggccgaccattatcaaca | |
| | gaacactccaatcggcgacggccctgtgctcctcccagacaaccattacctgtcc | |
| | acccagtctgccctgtctaaagatcccaacgaaaagagagaccacatggtcctgc | |
| | tggagtttgtgaccgctgctgggatcacacatggcatggacgagctgtacaagtg | |
| | agcggccgcggggttccagacatgataagatacattgatgagtttggacaaacca | |
| | caactagaatgcagtgaaaaaaatgctttatttgtgaaatttgtgatgctattgc | |
| | tttatttgtaaccattataagctgcaataaacaagttaacaacaacaattgcatt | |
| | cattttatgtttcaggttcagggggaggtgtgggaggttttttactagtaattca | |
| | tatttgcatgtcgctatgtgttctgggaaatcaccataaacgtgaaatgtctttg | |
| | gatttgggaatcttataagttctgtatgagaccactcggatccGTCCTGAACTCC | |
| | ATTATAATTCAAGAGATTATAATGGAGTTCAGGACttttttggaaaagcttgtcg | |
| | actagagctcgctgatcagcctcgactgtgccttctagttgccagccatctgttg | |
| | tttgcccctcccccgtgccttccttgaccctggaaggtgccactcccactgtcct | |
| | ttcctaataaaatgaggaaattgcatcgcattgtctgagtaggtgtcattctatt | |
| | ctggggggtggggtggggcaggacagcaagggggaggattgggaagacaatagca | |
| | ggcatgctggggagagatctaggaacccctagtgatggagttggccactccctct | |
| | ctgcgcgctcgctcgctcactgaggccgcccgggcaaagcccgggcgtcgggcga | |
| | cctttggtcgcccggcctcagtgagcgagcgagcgcgcagagagggagtggccaa | |
| | c | |
| 32 | ttggccactccctctctgcgcgctcgctcgctcactgaggccgggcgaccaaagg | AAV9.H1.shRNA- |
| | tcgcccgacgcccgggctttgcccgggcggcctcagtgagcgagcgagcgcgcag | CaV1.3-Human-5 |
| | agagggagtggccaactccatcactaggggttcctagatctgaattcggtgctag | |
| | caccctagttattaatagtaatcaattacggggtcattagttcatagcccatata | |
| | tggagttccgcgttacataacttacggtaaatggcccgcctggctgaccgcccaa | |
| | cgacccccgcccattgacgtcaataatgacgtatgttcccatagtaacgccaata | |
| | gggactttccattgacgtcaatgggtggactatttacggtaaactgcccacttgg | |
| | cagtacatcaagtgtatcatatgccaagtacgccccctattgacgtcaatgacgg | |
| | taaatggcccgcctggcattatgcccagtacatgaccttatgggactttcctact | |
| | tggcagtacatctacgtattagtcatcgctattaccatggtcgaggtgagcccca | |
| | cgttctgcttcactctccccatctcccccccctccccacccccaattttgtattt | |
| | atttattttttaattattttgtgcagcgatgggggcggggggggggggggggcgc | |
| | gcgccaggcggggcggggcggggcgaggggcggggcggggcgaggcggagaggtg | |
| | cggcggcagccaatcagagcggcgcgctccgaaagtttccttttatggcgaggcg | |
| | gcggcggcggcggccctataaaaagcgaagcgcgcggcgggcgggagtcgctgcg | |
| | acgctgccttcgccccgtgccccgctccgccgccgcctcgcgccgcccgccccgg | |
| | ctctgactgaccgcgttactcccacaggtgagcgggcgggacggcccttctcctc | |
| | cgggctgtaattagcgcttggtttaatgacggcttgtttcttttctgtggctgcg | |
| | tgaaagccttgaggggctccgggagggccctttgtgcgggggggagcggctcggg | |
| | gggtgcgtgcgtgtgtgtgtgcgtggggagcgccgcgtgcggcccgcgctgcccg | |
| | gcggctgtgagcgctgcgggcgcggcgcggggctttgtgcgctccgcagtgtgcg | |
| | cgaggggagcgcggccgggggcggtgccccgcggtgcggggggggctgcgagggg | |
| | aacaaaggctgcgtgcggggtgtgtgcgtgggggggtgagcagggggtgtgggcg | |
| | cggcggtcgggctgtaacccccccctgcacccccctccccgagttgctgagcacg | |
| | gcccggcttcgggtgcggggctccgtacggggcgtggcgcggggctcgccgtgcc | |
| | gggcggggggtggcggcaggtgggggtgccgggcggggcggggccgcctcgggcc | |
| | ggggagggctcgggggaggggcgcggcggcccccggagcgccggcggctgtcgag | |
| | gcgcggcgagccgcagccattgccttttatggtaatcgtgcgagagggcgcaggg | |
| | acttcctttgtcccaaatctgtgcggagccgaaatctgggaggcgccgccgcacc | |
| | ccctctagcgggcgcggggcgaagcggtgcggcgccggcaggaaggaaatgggcg | |
| | gggagggccttcgtgcgtcgccgcgccgccgtccccttctccctctccagcctcg | |
| | gggctgtccgcggggggacggctgccttcgggggggacggggcagggcggggttc | |
| | ggcttctggcgtgtgaccggcggctctagagcctctgctaaccatgttcatgcct | |
| | tcttctttttcctacagctcctgggcaacgtgctggttattgtgctgtctcatca | |
| | ttttggcaaagtattcctcgaagatctgctagcaggcgcgcggccgccgccacca | |
| | tgagcaagggcgaggaactgttcactggcgtggtcccaattctcgtggaactgga | |
| | tggcgatgtgaatgggcacaaattttctgtcagcggagagggtgaaggtgatgcc | |
| | acatacggaaagctcaccctgaaattcatctgcaccactggaaagctccctgtgc | |
| | catggccaacactggtcactaccctgacctatggcgtgcagtgcttttccagata | |
| | cccagaccatatgaagcagcatgactttttcaagagcgccatgcccgagggctat | |
| | gtgcaggagagaaccatctttttcaaagatgacgggaactacaagacccgcgctg | |
| | aagtcaagttcgaaggtgacaccctggtgaatagaatcgagctgaagggcattga | |
| | ctttaaggaggatggaaacattctcggccacaagctggaatacaactataactcc | |
| | cacaatgtgtacatcatggccgacaagcaaaagaatggcatcaaggtcaacttca | |
| | agatcagacacaacattgaggatggatccgtgcagctggccgaccattatcaaca | |
| | gaacactccaatcggcgacggccctgtgctcctcccagacaaccattacctgtcc | |
| | acccagtctgccctgtctaaagatcccaacgaaaagagagaccacatggtcctgc | |
| | tggagtttgtgaccgctgctgggatcacacatggcatggacgagctgtacaagtg | |
| | agcggccgcggggttccagacatgataagatacattgatgagtttggacaaacca | |
| | caactagaatgcagtgaaaaaaatgctttatttgtgaaatttgtgatgctattgc | |
| | tttatttgtaaccattataagctgcaataaacaagttaacaacaacaattgcatt | |
| | cattttatgtttcaggttcagggggaggtgtgggaggttttttactagtaattca | |
| | tatttgcatgtcgctatgtgttctgggaaatcaccataaacgtgaaatgtctttg | |
| | gatttgggaatcttataagttctgtatgagaccactcggatccGAGGATCTAAAG | |
| | GGCTACTTTCAAGAGAAGTAGCCCTTTAGATCCTCttttttggaaaagcttgtcg | |
| | actagagctcgctgatcagcctcgactgtgccttctagttgccagccatctgttg | |
| | tttgcccctcccccgtgccttccttgaccctggaaggtgccactcccactgtcct | |
| | ttcctaataaaatgaggaaattgcatcgcattgtctgagtaggtgtcattctatt | |
| | ctggggggtggggtggggcaggacagcaagggggaggattgggaagacaatagca | |
| | ggcatgctggggagagatctaggaacccctagtgatggagttggccactccctct | |
| | ctgcgcgctcgctcgctcactgaggccgcccgggcaaagcccgggcgtcgggcga | |
| | cctttggtcgcccggcctcagtgagcgagcgagcgcgcagagagggagtggccaa | |
| | c | |

What is claimed is:

1-23. (canceled)

24. An engineered vector-mediated system for reducing expression of a calcium channel, voltage-dependent, L type, alpha 1 D subunit (CaV1.3) protein in a target cell, the system comprising:
a. a nucleic acid expression construct encoding an interfering nucleic acid molecule having a nucleotide sequence complementary to a target sequence within a gene encoding the CaV1.3 protein to reduce the expression of the CaV1.3 protein; and
b. an rAAV vector encapsidating the nucleic acid expression construct for delivering the nucleic acid expression construct to the target cell;
wherein the-CaV1.3 protein has an amino acid sequence encoded by a nucleotide sequence of a human Cacnald gene, wherein the nucleotide sequence encoding the human Cacnald gene has about 75% or more, 85% or more, 95% or more, or 100% sequence identity with the nucleic acid sequence of SEQ ID NO: 12.

25. The vector-mediated system of claim 24, wherein the expression construct expresses an shRNA molecule comprising a nucleotide sequence complementary to a target a sequence within a gene encoding the CaV1.3 protein, operably linked to a promoter.

26. The vector-mediated system of claim 24, wherein the vector provides continuous, high-potency, and target-selective mRNA-level silencing of striatal CaV1.3 channels.

27. An rAAV vector for reducing the expression of a CaV1.3 protein in a target cell, the vector comprising a nucleic acid expression construct for expressing a protein expression modification system,
wherein the protein expression modification system is engineered to reduce the expression of the CaV1.3 protein; and
wherein the rAAV vector comprises a nucleotide sequence having about 75% or more, 85% or more, 95% or more, or 100% sequence identity with a nucleotide sequence selected from SEQ ID NO: 27-32.

28. The rAAV vector of claim 27, wherein the nucleic acid expression construct comprises a nucleotide sequence encoding the shRNA operably linked to a promoter for expressing the shRNA sequence, wherein the shRNA has a nucleotide sequence complementary to a target sequence within a gene encoding the CaV1.3 protein.

29. (canceled)

30. The rAAV vector of claim 28, wherein the target sequence within the gene encoding the CaV1.3 protein is selected from SEQ ID NOs: 1-8.

31. The rAAV vector of claim 28, wherein the shRNA comprises a nucleotide sequence selected from SEQ ID NO: 9, SEQ ID NOs: 14-19, or any combination thereof.

32. A method of treating a levodopa-induced dyskinesia (LID) in a subject in need thereof, the method comprising reducing the expression of a CaV1.3 protein in a target cell by administering to the subject a therapeutically effective amount of a composition comprising the system of claim 24.

33. The method of claim 32, wherein the subject has Parkinson's disease or is at risk of developing Parkinson's Disease (PD).

34. The method of claim 32, wherein the method prevents induction of dyskinesia in a subject undergoing dopamine agonist (DA) replacement therapy or expected to undergo DA replacement therapy.

35. The method of claim 32, wherein the method reduces, reverses, or eliminates dyskinesia in a subject undergoing DA replacement therapy.

36. The method of claim 32, wherein the method reverses dyskinesia in a subject undergoing DA replacement therapy.

37. (canceled)

38. The method of claim 32, wherein the system is administered to the subject after a 1-week temporary withdrawal of DA replacement therapy.

39-40.

41. A method of improving the response to a DA replacement therapy in a subject in need thereof, the method comprising administering to the subject a therapeutically effective amount of a composition comprising an engineered genetic system of claim 24 for reducing the expression of a CaV1.3 protein in a target cell.

42. A method of protecting neurons from damage in a subject in need thereof, the method comprising administering to the subject a therapeutically effective amount of a composition comprising an engineered genetic system of claim 24 for reducing the expression of a CaV1.3 protein in a target cell.

43. A method of slowing progression of Parkinson's disease by providing protection against death or dysfunction of substantia nigra dopamine neurons in a subject in need thereof, the method comprising administering to the subject a therapeutically effective amount of a composition comprising an engineered genetic system of claim 24 for reducing the expression of a CaV1.3 protein in a target cell.

44. One or more nucleic acid constructs encoding the engineered vector-mediated system of claim 24 for reducing the expression of a CaV1.3 protein in a target cell.

45. (canceled)

46. A kit comprising one or more engineered vector-mediated system of claim 24, for reducing the expression of a CaV1.3 protein in a target cell.

47. (canceled)


SEQUENCE LISTING (32 sequences)

SEQ ID NO: 1 - DNA, 19 bp, Rattus norvegicus
cgaggcaaactatgcaaga

SEQ ID NO: 2 - DNA, 20 bp, Rattus norvegicus
actatgcaagaggcaccaga

SEQ ID NO: 3 - DNA, 19 bp, Rattus norvegicus
gcgtcagtgtgtggaatat

SEQ ID NO: 4 - DNA, 19 bp, Rattus norvegicus
ggccattgctgtagacaat

SEQ ID NO: 5 - DNA, 19 bp, Rattus norvegicus
cgtgccctcttctgtttat

SEQ ID NO: 6 - DNA, 19 bp, Rattus norvegicus
ctcctcgcctttcgaatat

SEQ ID NO: 7 - DNA, 19 bp, Rattus norvegicus
ccctgaagatgattctaat

SEQ ID NO: 8 - DNA, 19 bp, Rattus norvegicus
cccgctattatgaaactta

SEQ ID NO: 9 - DNA, 19 bp, Rattus norvegicus
gaagaggcgcggccaagac

SEQ ID NO: 10 - DNA, 19 bp, Rattus norvegicus
caacaagatgaagagcacc

SEQ ID NO: 11 - DNA, 104 bp, Homo sapiens
aattcatatttgcatgtcgctatgtgttctgggaaatcaccataaacgtgaaatgtctttggatttgggaatcttataagttctgtatgagacactcggatccg

SEQ ID NO: 12 - DNA, 1623 bp, Homo sapiens
atgatgatgatgatgatgatgaaaaaaatgcagcatcaacggcagcagcaagcggaccacgcgaacgaggcaaactatgcaagaggcaccagactccctctttctggtgaaggaccaacttctcagccgaatagctccaagcaaactgtcctgtcttggcaagctgcaatcgatgctgctagacaggccaaggctgcccaaactatgagcacctctgcaccccgacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactctagcggcaagtctggaggctttgatgtcaaagccctccgtgcctttcgagtgttgcgaccacttcgactagtgtcaggagtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaacggacgccagtgtaccgccaatggcacggaatgtaggagtggctgggtcggcccgaacggaggcatcaccaactttgataactttgcctttgctatgctcactgtgtttcagtgcatcaccatggagggctggacagatgtgctctactgggtaaatgatgcgataggatgggaatggccatgggtgtattttgttagtctcatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatcccgagaacgaggaagaaggaggagaggaaggcaaacgaaatactagcatgcccaccagtgagactgagtctgtgaacacagagaacgtcagcggcgaaggcgagacccgaggctgctgtggaagtctctggtgctggtggagacggagaggcgcggccaaggcggggccctctgggtgtcggcggtggggtcaagccatctcaaaatccaaactcagccgacgctggcgtcgctggaaccgattcaatcgcagaagatgtagggccgccgtgaag

SEQ ID NO: 13 - protein, 2161 aa, Homo sapiens
MMMMMMMKKMQHQRQQQADHANEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWMNDAMGFELPWVYFVSLVIFGSFFVLNLVLGVLSGEFSKEREKAKARGDFQKLREKQQLEEDLKGYLDWITQAEDIDPENEEEGGEEGKRNTSMPTSETESVNTENVSGEGENRGCCGSLCQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSLSNLVASLLNSMKSIASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADPTESENVPVPTATPGNSEESNRISITFFRLFRVMRLVKLLSRGEGIRTLLWTFIKSFQALPYVALLIAMLFFIYAVIGMQMFGKVAMRDNNQINRNNNFQTFPQAVLLLFRCATGEAWQEIMLACLPGKLCDPESDYNPGEEYTCGSNFAIVYFISFYMLCAFLIINLFVAVIMDNFDYLTRDWSILGPHHLDEFKRIWSEYDPEAKGRIKHLDVVTLLRRIQPPLGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKRTRYYETYIRSDSGDEQLPTICREDPEIHGYFRDPHCLGEQEYFSSEECYEDDSSPTWSRQNYGYYSRYPGRNIDSERPRGYHHPQGFLEDDDSPVCYDSRRSPRRRLLPPTPASHRRSSFNFECLRRQSSQEEVPSSPIFPHRTALPLHLMQQQIMAVAGLDSSKAQKYSPSHSTRSWATPPATPPYRDWTPCYTPLIQVEQSEALDQVNGSLPSLHRSSWYTDEPDISYRTFTPASLTVPSSFRNKNSDKQRSADSLVEAVLISEGLGRYARDPKFVSATKHEIADACDLTIDEMESAASTLLNGNVRPRANGDVGPLSHRQDYELQDFGPGYSDEEPDPGRDEEDLADEMICITTL

SEQ ID NO: 14 - DNA, 47 bp, Artificial Sequence
  SYNTHESIZED
gaagaggcgcggccaagacttcaagagagtcttggccgcgcctcttc

SEQ ID NO: 15 - DNA, 48 bp, Homo sapiens
gcgaggcaaactatgcaagattcaagagatcttgcatagtttgcctcg

SEQ ID NO: 16 - DNA, 47 bp, Artificial Sequence
  SYNTHESIZED
gaggaaggcaaacgaaatattcaagagatatttcgtttgccttcctc

SEQ ID NO: 17 - DNA, 48 bp, Artificial Sequence
  SYNTHESIZED
gccctgaagatgattctaatttcaagagaattagaatcatcttcaggg

SEQ ID NO: 18 - DNA, 47 bp, Artificial Sequence
  SYNTHESIZED
gtcctgaactccattataattcaagagattataatggagttcaggac

SEQ ID NO: 19 - DNA, 47 bp, Artificial Sequence
  SYNTHESIZED
gaggatctaaagggctactttcaagagaagtagccctttagatcctc

SEQ ID NO: 20 - DNA, 11 bp, Artificial Sequence
  SYNTHESIZED
ttttttggaaa

SEQ ID NO: 21 - DNA, 162 bp, Artificial Sequence
  SYNTHESIZED
aattcatatttgcatgtcgctatgtgttctgggaaatcaccataaacgtgaaatgtctttggatttgggaatcttataagttctgtatgagaccactcggatccgaagaggcgcggccaagacttcaagagagtcttggccgcgcctcttcttttttggaaa

SEQ ID NO: 22 - DNA, 163 bp, Artificial Sequence
  SYNTHESIZED
aattcatatttgcatgtcgctatgtgttctgggaaatcaccataaacgtgaaatgtctttggatttgggaatcttataagttctgtatgagaccactcggatccgcgaggcaaactatgcaagattcaagagatcttgcatagtttgcctcgttttttggaaa

SEQ ID NO: 23 - DNA, 162 bp, Artificial Sequence
  SYNTHESIZED
aattcatatttgcatgtcgctatgtgttctgggaaatcaccataaacgtgaaatgtctttggatttgggaatcttataagttctgtatgagaccactcggatccgaggaaggcaaacgaaatattcaagagatatttcgtttgccttcctcttttttggaaa

SEQ ID NO: 24 - DNA, 162 bp, Artificial Sequence
  SYNTHESIZED
aattcatatttgcatgtcgctatgtgttctgggaaatcaccataaacgtgaaatgtctttggatttgggaatcttataagttctgtatgagaccactcggatccgtcctgaactccattataattcaagagattataatggagttcaggacttttttggaaa

SEQ ID NO: 25 - DNA, 162 bp, Artificial Sequence
  SYNTHESIZED
aattcatatttgcatgtcgctatgtgttctgggaaatcaccataaacgtgaaatgtctttggatttgggaatcttataagttctgtatgagaccactcggatccgaggatctaaagggctactttcaagagaagtagccctttagatcctcttttttggaaa

SEQ ID NO: 26 - DNA, 163 bp, Artificial Sequence
  SYNTHESIZED
aattcatatttgcatgtcgctatgtgttctgggaaatcaccataaacgtgaaatgtctttggatttgggaatcttataagttctgtatgagaccactcggatccgccctgaagatgattctaatttcaagagaattagaatcatcttcagggttttttggaaa

SEQ ID NO: 27 - DNA, 3411 bp, Artificial Sequence
  SYNTHESIZED
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggttccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaattcatatttgcatgtcgctatgtgttctgggaaatcaccataaacgtgaaatgtctttggatttgggaatcttataagttctgtatgagaccactcggatccgaagaggcgcggccaagacttcaagagagtcttggccgcgcctcttcttttttggaaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 28 - DNA, 3412 bp, Artificial Sequence
  SYNTHESIZED
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggttccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaattcatatttgcatgtcgctatgtgttctgggaaatcaccataaacgtgaaatgtctttggatttgggaatcttataagttctgtatgagaccactcggatccgcgaggcaaactatgcaagattcaagagatcttgcatagtttgcctcgttttttggaaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 29 - DNA, 3411 bp, Artificial Sequence
  SYNTHESIZED
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggttccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaattcatatttgcatgtcgctatgtgttctgggaaatcaccataaacgtgaaatgtctttggatttgggaatcttataagttctgtatgagaccactcggatccgaggaaggcaaacgaaatattcaagagatatttcgtttgccttcctcttttttggaaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 30 - DNA, 3412 bp, Artificial Sequence
  SYNTHESIZED
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggttccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaattcatatttgcatgtcgctatgtgttctgggaaatcaccataaacgtgaaatgtctttggatttgggaatcttataagttctgtatgagaccactcggatccgccctgaagatgattctaatttcaagagaattagaatcatcttcagggttttttggaaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 31 - DNA, 3411 bp, Artificial Sequence
  SYNTHESIZED
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggttccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaattcatatttgcatgtcgctatgtgttctgggaaatcaccataaacgtgaaatgtctttggatttgggaatcttataagttctgtatgagaccactcggatccgtcctgaactccattataattcaagagattataatggagttcaggacttttttggaaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 32 - DNA, 3411 bp, Artificial Sequence
  SYNTHESIZED
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtgctagcaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagtattcctcgaagatctgctagcaggcgcgcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcggggttccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactagtaattcatatttgcatgtcgctatgtgttctgggaaatcaccataaacgtgaaatgtctttggatttgggaatcttataagttctgtatgagaccactcggatccgaggatctaaagggctactttcaagagaagtagccctttagatcctcttttttggaaaagcttgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac